(19) United States
(12) Patent Application Publication
(10) Pub. No.: US 2026/0053982 A1
(43) Pub. Date: Feb. 26, 2026

(54) SCAFFOLD MATERIALS FOR ARTIFICIAL DERMAL LAYER

(71) Applicant: VITROLABS INC, Milpitas, CA (US)

(72) Inventors: Gabriel MICKEL, Milpitas, CA (US); Rui TANG, Milpitas, CA (US); Craig HALBERSTADT, Milpitas, CA (US); Ingvar HELGASON, Milpitas, CA (US)

(21) Appl. No.: 19/105,603

(22) PCT Filed: Aug. 23, 2023
(86) PCT No.: PCT/US2023/030893

§ 371 Date: Feb. 21, 2025

Related U.S. Application Data
(60) Provisional application No. 63/400,704, filed on Aug. 24, 2022.
(60) Provisional application No. 63/515,982, filed on Jul. 27, 2023.

(51) Int. Cl. A61L 27/24; A61L 27/16; A61L 27/18; A61L 27/20; A61L 27/36; A61L 27/44; A61L 27/58; C12N 5/077; D04H 1/425; D04H 1/4318; D04H 1/4334; D04H 1/435; D04H 1/46

(52) U.S. Cl. CPC A61L 27/24; A61L 27/16; A61L 27/18; A61L 27/20; A61L 27/362; A61L 27/3683; A61L 27/44; A61L 27/58; C12N 5/0656; D04H 1/425; D04H 1/4318; D04H 1/4334; D04H 1/435; D04H 1/46; A61L 2430/34; C12N 2510/04; C12N 2533/30; C12N 2533/40; C12N 2533/90; D10B 2401/10; D10B 2509/00

(57) ABSTRACT

Disclosed herein are compositions for use as scaffold materials for artificial dermal layers. Disclosed herein are methods for the manufacture and processing of scaffold materials for artificial dermal layers. Disclosed herein are scaffold materials for use supporting immortalized fibroblasts.

### CROSS-REFERENCE

This Application claims the benefit of U.S. Provisional Patent Application No. 63/400,704 filed on Aug. 24, 2022, and U.S. Provisional Patent Application No. 63/515,982 filed on Jul. 27, 2023, each of which are incorporated herein by reference in their entirety.

### SUMMARY

Disclosed herein in some embodiments, is a composition comprising a scaffold in contact with an extracellular matrix comprising a collagen, wherein a scaffold can comprise a non-woven, needle-punched material. In some embodiments, disclosed herein is a composition comprising a scaffold in contact with an extracellular matrix comprising a collagen, wherein the scaffold can comprise a three-dimensional woven material. In some embodiments, disclosed herein is a composition comprising a scaffold in contact with an extracellular matrix comprising a collagen, wherein a scaffold can comprise a polycaprolactone (PCL), a polylactic acid (PLA), a polylactic and polyglycolic acid (PLGA), a polyethylene terephthalate (PET), a nylon, a polyethylene (PE), a polyethylene furanoate (PEF), a polypropylene (PP), a polyvinyl alcohol (PVA), a cotton, a bast fiber, a viscose, a modal, a lyocell, a plant based protein fiber, a biobased material, a viscose, a cellulose, an alginate fiber, a thermoplastic starch, or any combination thereof. In some embodiments, disclosed herein is a composition comprising an at least partially coated scaffold in contact with an extracellular matrix comprising a collagen, wherein a scaffold is at least partially coated with a coating comprising a matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, or any combination thereof. Disclosed herein in some embodiments, is a composition comprising a dissolvable scaffold in contact with an extracellular matrix comprising a collagen, wherein a scaffold is in contact with a solvent that can dissolve the scaffold. In some embodiments, the scaffold can comprise a polycaprolactone (PCL), a polylactic acid (PLA), a polylactic and polyglycolic acid (PLGA), a polyethylene terephthalate (PET), a nylon, a polyethylene (PE), a polyethylene furanoate (PEF), a polypropylene (PP), a polyvinyl alcohol (PVA), a cotton, a bast fiber, a viscose, a modal, a lyocell, a plant based protein fiber, a biobased material, a viscose, a cellulose, an alginate fiber, a thermoplastic starch, or any combination thereof. In some embodiments, the scaffold can comprise a non-woven configuration that can create a first plurality of pores. In some embodiments, a first plurality of pores can comprise an average pore size of about 30-70 μm. In some embodiments, a first plurality of pores can comprise an average pore size of about 80-120 μm. In some embodiments, a first plurality of pores can comprise an average pore size of about 50 μm or about 100 μm. In some embodiments, a scaffold can comprise a second plurality of pores produced by a needle punch. In some embodiments, a scaffold can be at least partially coated with a coating comprising a matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, or any combination thereof. In some embodiments, the scaffold is at least partially coated with a coating comprising a Matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, GTMAC, carbohydrate-binding module, a cellulose binding domain, a starch binding domain, or a combination thereof. In some embodiments, the scaffold is at least partially coated with a carbohydrate-binding module. In some embodiments, the carbohydrate-binding module is a cellulose-binding domain. In some embodiments, the scaffold comprises cellulose. In some embodiments, the carbohydrate-binding module is a starch-binding module. In some embodiments, the scaffold comprises starch. In some embodiments, the carbohydrate-binding module is associated with an enzyme. In some embodiments, the enzyme does not hydrolyze the scaffold. In some embodiments, a coating can comprise a modification. In some embodiments, a modification can comprise a reductive modification, an additive modification, or a combination thereof. In some embodiments, the modification comprises hydrolysis. In some embodiments, the modification exposes chemically active groups comprising hydroxyls, carboxylic acids, ketones, or a combination thereof. In some embodiments, the modification comprises oxidation. In some embodiments, the oxidation occurs with sodium periodate. In some embodiments, the scaffold comprises an amine, carboxylic acid, sulfate, aldehyde, hydrazide, sulfhydryl, diazirine, aryl-azide, acrylate, and epoxide. In some embodiments, the scaffold is at least partially coated with a coating comprising GTMAC. In some embodiments, the scaffold comprises primary amines. In some embodiments, at least partially coating with a coating comprising GTAMC increases the surface charge of the scaffold. In some embodiments, the collagen is associated with the scaffold. In some embodiments, the collagen is associated with the scaffold through non-specific adsorption. In some embodiments, the collagen is associated with the scaffold through Van der Waals interactions. In some embodiments, the collagen is associated with the scaffold through hydrogen bonding. In some embodiments, the collagen is associated with the scaffold through depletion interactions. In some embodiments, the collagen is associated with the scaffold through electrostatic interactions. In some embodiments, the modification of the scaffold increases a strength of a non-specific adsorption of the collagen to the scaffold. In some embodiments, the collagen is associated with the scaffold through covalent interactions. In some embodiments, the scaffold comprises a carbodiimide or an N-hydroxysuccinimide ester (NHS ester). In some embodiments, the collagen comprises a carbodiimide or an N-hydroxysuccinimide ester (NHS ester). In some embodiments, the carbodiimide is N,N′-Dicyclohexylcarbodiimide (DCC) or 1-Ethyl-3-(3-dimethylaminopropyl)carbodiimide (EDC). In some embodiments, the collagen is associated with the scaffold through EDC/NHS coupling. In some embodiments, the scaffold comprises an azide or an alkyne. In some embodiments, the collagen comprises an azide or alkyne. In some embodiments, the collagen is associated with the scaffold through a click chemistry reaction. In some embodiments, the scaffold comprises a Michael donor or a Michael acceptor. In some embodiments, the collagen comprises a Michael donor or a Michael acceptor. In some embodiments, the collagen is associated with the scaffold through coupling of the Michael donor and the Michael acceptor. In some embodiments, the Michael donor comprises an enolate. In some embodiments, the Michael acceptor comprises an α,β-unsaturated carbonyl. In some embodiments, the scaffold comprises a thiol or maleimide. In some embodiments, the collagen comprises a thiol or maleimide. In some embodiments, the collagen is associated with the scaffold through coupling of the thiol and maleimide. In some embodiments, the collagen is associated with the scaffold in the presence of (sulfosuccinimidyl 4-(N-maleimidomethyl)cyclohexane-1-carboxylate) (sulfo-SMCC), wherein the scaffold and collagen comprise a thiol. In some embodiments, the collagen is associated with the scaffold through a Maillard reaction.

In some embodiments, the compositions provided herein comprise a scaffold. In some embodiments, a scaffold disclosed herein can be a dissolvable scaffold, wherein the scaffold can be in contact with a solvent that can dissolve the scaffold. In some embodiments, a temperature of the solvent is below the boiling point of the solvent. In some embodiments, the solvent can be at a temperature of about 40° C. to about 50° C. In some embodiments, the scaffold can comprise a thermoplastic polymer. In some embodiments, a thermoplastic polymer can comprise a polyvinyl alcohol (PVA), and wherein a solvent can comprise a water. In some embodiments, the thermoplastic polymer comprises a polyvinyl alcohol (PVA) or polyvinyl alcohol (PVOH). In some embodiments, the thermoplastic polymer comprises polylactic acid (PLA). In some embodiments, the solvent comprises water. In some embodiments, the solvent comprises an organic solvent. In some embodiments, the organic solvent comprises acetone, benzylamine, or ethyl acetate. In some embodiments, a scaffold can comprise a polylactic acid (PLA), and wherein a solvent can comprise a benzyl group, an ethyl group, a haloalkane, or a combination thereof. In some embodiments, a solvent can comprise a benzylamine. In some embodiments, the scaffold is dissolved over time through hydrolytic degradation. In some embodiments, the scaffold is degraded over time through hydrolytic degradation in a neutral aqueous solution. In some embodiments, the scaffold comprises a polyhydroxyalkanoate (PHA), polyhydroxybutyrate (PHB), polycaprolactone (PCL), polylactic acid (PLA), or oxidized alginate. In some embodiments, the solvent comprises a dissolution agent. In some embodiments, the dissolution agent comprises ethylenediaminetetraacetic acid (EDTA). In some embodiments, the scaffold comprises an alginate. In some embodiments, the dissolution agent comprises a strong acid. In some embodiments, the strong acid comprises hydrochloric acid, nitric acid, hydroiodic acid, perchloric acid, chloric acid, or a combination thereof. In some embodiments, the dissolution agent comprises a strong base. In some embodiments, the strong base comprises lithium hydroxide, sodium hydroxide, potassium hydroxide, calcium hydroxide, strontium hydroxide, barium hydroxide, or a combination thereof. In some embodiments, the dissolution agent comprises an oxidizing agent. In some embodiments, the oxidizing agent partially or completely degrades the scaffold. In some embodiments, the oxidizing agent is sodium periodate.

In some embodiments, the compositions provided herein comprise a scaffold. In some embodiments, a scaffold disclosed herein can comprise a biobased material and a solvent can comprise an enzyme that degrades the biobased material. In some embodiments, a scaffold can comprise a cellulose and a solvent can comprise a cellulase. In some embodiments, the scaffold comprises an ester-containing polymer. In some embodiments, the solvent comprises an esterase. In some embodiments, the solvent comprises a lipase. In some embodiments, the scaffold comprises a calcium alginate. In some embodiments, the solvent comprises an alginate lyase.

In some embodiments, the compositions provided herein comprise a scaffold. In some embodiments, a scaffold disclosed herein can comprise a three-dimensional woven material. In some embodiments, a three-dimensional woven material can comprise a spacer fabric. In some embodiments, a spacer fabric can comprise a front face in contact with a filler, and a back face in contact with a filler, wherein a filler separates a front face from a back face. In some embodiments, a separation of a front face from a back face by a filler can create a gap to allow nutrient entry, waste removal, cell attachment, cell growth, or any combination thereof. In some embodiments, a three-dimensional woven material can comprise a pile weave. In some embodiments, a three-dimensional woven material can comprise a terry, a frieze, a velvet, a corduroy, a velveteen, or any combination thereof. In some embodiments, a scaffold can comprise multiple layers of scaffold material. In some embodiments, multiple layers of scaffold material can comprise a combination of different form factors. In some embodiments, different form factors can comprise a non-woven material, a woven material, a needle-punched material, a three-dimensional structure, or any combination thereof. In some embodiments, a scaffold can comprise a three-layered composite material, wherein a three-layered composite material can comprise two outer layers and one inner layer. In some embodiments, two outer layers can comprise a surface layer characteristic, and an inner layer can comprise a bulk property. In some embodiments, multiple layers can comprise multiple layers of a thin material. In some embodiments, multiple layers can be fused together, held together by entanglement, laminated together, stitched together, glued together, woven together, printed together, or any combination thereof. In some embodiments, a composition disclosed herein further can comprise an isolated animal cell in contact with a scaffold. In some embodiments, an isolated animal cell can be an isolated animal fibroblast or fibroblast-like cell. In some embodiments, an extracellular matrix can be produced by an isolated animal fibroblast or fibroblast-like cell. In some embodiments, an isolated animal cell can be an immortalized isolated animal cell. In some embodiments, an immortalized isolated animal cell can be grown past a Hayflick limit. In some embodiments, an immortalized isolated animal cell can be grown past about 40 cell divisions, about 50 cell divisions, or about 60 cell divisions. In some embodiments, an isolated animal cell can be a bovine or porcine cell. In some embodiments, an isolated animal cell can comprise a human cell. In some embodiments, a composition disclosed herein can be at least partially decellularized. In some embodiments, at least partially decellularized can comprise substantially no intact cells being present in a composition. In some embodiments, a scaffold further can comprise a polyglycolic acid (PGA), a polybutylene succinate (PBS), a bioabsorbable synthetic polymer, a cellulose, an acetate, an acrylic, a fiber, a linen, a rayon, a velvet, a modacrylic, an olefin polyester, a saran, a vinyon, a wool, a jute, a hemp, a bamboo, a flax, or any combination thereof. In some embodiments, a scaffold further can comprise a bio-based nylon, a bio-based PET, a bio-based PEF, a bio-based polylactic acid (PLA) or any combination thereof. In some embodiments, a scaffold can comprise a nylon 1,6, a nylon 4,6, a nylon 510, a nylon 5,6, a nylon 5,12, a nylon 6, a nylon 6,6, a nylon 11, a nylon 10.10, a nylon 12, or any combination thereof. In some embodiments, a scaffold can comprise a bast fiber, wherein a bast fiber can comprise a flax, a hemp, a linen, a jute, a ramie, a kenaf, a sisal, or any combination thereof. In some embodiments, a scaffold can comprise a thickness of from about 0.1 mm to about 4 mm. In some embodiments, a scaffold can comprise a thickness of from about 1 mm to about 3 mm. In some embodiments, a scaffold can comprise a thickness of about 1 mm. In some embodiments, a scaffold can comprise a thickness of about 2 mm. In some embodiments, a scaffold can comprise a thickness of about 3 mm. In some embodiments, a scaffold can comprise fibers comprising a dtex of about 6.7 dtex. In some embodiments, a scaffold can comprise fibers comprising a diameter of from about 1 μm to about 100 μm. Disclosed herein in some embodiments, is a method of making a composition disclosed herein, comprising seeding an isolated animal fibroblast or fibroblast-like cell onto a scaffold to form a composition. In some embodiments, a scaffold can comprise a thermoplastic polymer which can be subsequently substantially removed from an extracellular matrix prior to a tanning. In some embodiments, a thermoplastic polymer can comprise a polyvinyl alcohol (PVA), and a method disclosed herein can comprise substantially removing the PVA prior to a tanning by contacting the PVA with water having a temperature of about 18° C. to about 90° C. In some embodiments, a thermoplastic polymer can comprise a Polylactic acid (PLA) and a method disclosed herein can comprise substantially removing the PLA prior to a tanning by contacting the PLA with a solvent to remove the PLA. In some embodiments, a solvent can comprise a benzyl group, an ethyl group, a haloalkane, a benzylamine, or any combination thereof. In some embodiments, a scaffold can comprise a dissolvable scaffold which can be substantially removed by contacting a scaffold with a solvent. In some embodiments, a method of making a scaffold can comprise needle punching a scaffold using a needle loom to entangle fibers in a non-woven scaffold material. In some embodiments, a needle loom can comprise a barbed needle. In some embodiments, a needle punching can create pores in a scaffold material. In some embodiments, a method of making a scaffold can comprise at least partially coating a scaffold with a coating comprising a matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, or any combination thereof.

Disclosed herein in some embodiments, is a method of making any of the compositions provided herein comprising at least partially coating the scaffold with a coating comprising a Matrigel, a vitronectin, a fibronectin, a protein extract from a soy, a protein extract from a pea, a protein extract from a corn, a peptide generated from synthesis, an RNA-binding glycine-rich (RBG) protein, a synthetic protein, an RGD peptide, a polylysine, a polyarginine, a polyornithine, a recombinant protein, an oligomer, a polymer, GTAMC, carbohydrate-binding module, a cellulose binding domain, a starch binding domain, or a combination thereof. In some embodiments, at least partially coating the scaffold with GMATC, providing a primary amine on the scaffold. In some embodiments, the carbohydrate-binding module is associated with an enzyme. In some embodiments, the enzyme does not hydrolyze the scaffold. In some embodiments, the carbohydrate-binding module is a cellulose binding domain. In some embodiments, the carbohydrate-binding module is a starch binding domain. In some embodiments, the scaffold comprises a modification. In some embodiments, the modification comprises addition of an amine, carboxylic acid, sulfate, aldehyde, hydrazide, sulfhydryl, diazirine, aryl-azide, acrylate, and epoxide. In some embodiments, the modification comprises hydrolysis, oxidation, enzymolysis, acidification, or basification. In some embodiments, the modification exposes one or more active groups comprising hydroxyls, carboxyls, ketones, or a combination thereof. In some embodiments, the method further comprises seeding an isolated animal fibroblast or fibroblast-like cell onto the scaffold to form the composition. In some embodiments, the composition comprises a collagen. In some embodiments, the collagen, fibroblast, and/or fibroblast-like cell is associated with the scaffold. In some embodiments, the collagen is associated with the scaffold through specific adsorption. In some embodiments, specific adsorption comprises interaction with the carbohydrate-binding module. In some embodiments, the carbohydrate-binding module comprises a cellulose binding domain or a starch binding domain. In some embodiments, the collagen, fibroblast, and/or fibroblast-like cell is associated with the scaffold through non-specific adsorption. In some embodiments, the collagen is associated with the scaffold through non-specific adsorption. In some embodiments, the isolated animal fibroblast and/or fibroblast-like cell are associated with the scaffold through non-specific adsorption. In some embodiments, the non-specific adsorption comprises Van der Waals interactions. In some embodiments, the non-specific adsorption comprises hydrogen bonding. In some embodiments, the non-specific adsorption comprises depletion interactions. In some embodiments, the non-specific adsorption comprises electrostatic interactions. In some embodiments, modification of the scaffold increases non-specific adsorption of the collagen, isolated animal fibroblast, and/or fibroblast-like cell to the scaffold. In some embodiments, the collagen, isolated animal fibroblast, and/or fibroblast-like cell is associated with the scaffold through covalent interactions. In some embodiments, the collagen is associated with the scaffold through covalent interactions. In some embodiments, the isolated animal fibroblast, and/or fibroblast-like cell is associated with the scaffold through covalent interactions. In some embodiments, the scaffold comprises a carbodiimide or an N-hydroxysuccinimide ester (NHS ester). In some embodiments, the collagen, isolated animal fibroblast, and/or fibroblast-like cell comprises a carbodiimide or an N-hydroxysuccinimide ester (NHS ester). In some embodiments, the collagen is associated with the scaffold through EDC/NHS coupling. In some embodiments, the fibroblast and/or fibroblast-like cell is associated with the scaffold through EDC/NHS coupling. In some embodiments, the scaffold comprises an azide or an alkyne. In some embodiments, the collagen, isolated animal fibroblast, and/or fibroblast-like cell comprises an azide or alkyne. In some embodiments, the collagen is associated with the scaffold through a click chemistry reaction. In some embodiments, the collagen is associated with the scaffold through a click chemistry reaction. In some embodiments, the fibroblast and/or fibroblast-like cell is associated with the scaffold through a click chemistry reaction. In some embodiments, wherein the scaffold comprises a Michael donor or a Michael acceptor. In some embodiments, the collagen, fibroblast, and/or fibroblast-like cell comprises a Michael donor or a Michael acceptor. In some embodiments, the collagen is associated with the scaffold through coupling of the Michael donor and the Michael acceptor. In some embodiments, the fibroblast and/or fibroblast-like cell is associated with the scaffold through coupling of the Michael donor and the Michael acceptor. In some embodiments, the Michael donor comprises an enolate. In some embodiments, the Michael acceptor comprises an α,β-unsaturated carbonyl. In some embodiments, the scaffold comprises a thiol or a maleimide. In some embodiments, the collagen, fibroblast, and/or fibroblast-like cell comprises a thiol or a maleimide. In some embodiments, the collagen is associated with the scaffold through coupling of the thiol and maleimide. In some embodiments, the fibroblast and/or fibroblast-like cell is associated with the scaffold through coupling of the thiol and maleimide. In some embodiments, the collagen is associated with the scaffold in the presence of sulfo-SMCC. In some embodiments, the scaffold and collagen both comprise a thiol. In some embodiments, the collagen, fibroblast, and/or fibroblast-like cell is associated with the scaffold through a Maillard reaction. In some embodiments, the collagen is associated with the scaffold through a Maillard reaction.

Disclosed herein in some embodiments, is a method comprising transplanting a composition disclosed herein onto a patient in need of a skin graft for a treatment of lost or damaged skin. In some embodiments, a patient can have lacerations, contusions, lesions, sores, burns, wounds, surgical wounds, surgically removed skin, necrotic skin, or any combination thereof. Disclosed herein in some embodiments, is a method comprising tanning a composition disclosed herein to produce a cruelty free leather. Disclosed herein in some embodiments, is a method of using a cruelty-free leather disclosed herein as a substitute for a traditional leather in a leather article. Disclosed herein is a leather article comprising a cruelty-free leather as disclosed herein. In some embodiments, a leather article can comprise a watch strap, a belt, a suspender, a packaging, a shoe, a boot, a footwear, a glove, a clothing, a bag, a clutch, a purse, a coin purse, a billfold, a key pouch, a credit card case, a pen case, a backpack, a case, a wallet, a saddle, a harness, a whip, a luggage, a travel good, a rucksack, a portfolio, a document bag, a briefcase, an attache case, a pet article, a leash, a collar, a hunting and fishing article, a gun case, a cutlery case, a holster for a fire arm, a stationary article, a writing pad, a book cover, a camera case, a spectacle case, a cigarette case, a cigar case, a jewel case, a mobile phone holster, a sport article, a ball, a basketball, a soccer ball, a football, or any combination thereof. In some embodiments, a clothing can comprise a top, a bottom, an outerwear, or any combination thereof. In some embodiments, a bag can comprise a handbag with or without a shoulder strap. In some embodiments, a luggage can comprise a trunk, a suitcase, a travel bag, a beauty case, a toilet kit, or any combination thereof. Disclosed herein in some embodiments, is a method of using a composition as disclosed herein for a treatment of a disease or ailment. Disclosed herein in some embodiments, is a kit comprising a composition as disclosed herein or a leather article as disclosed herein.

Disclosed herein is a method comprising: 1) seeding isolated animal fibroblasts or fibroblast-like cells onto a scaffold to form a cell layer on a scaffold, wherein the scaffold comprises a polycaprolactone (PCL), a polylactic acid (PLA), a polylactic and polyglycolic acid (PLGA), a polyethylene terephthalate (PET), a Nylon, a polyethylene (PE), a polyethylene furanoate (PEF), a polypropylene (PP), a polyvinyl alcohol (PVA), a cotton, a bast fiber, a viscose, a modal, a lyocell, a plant based protein fiber, an alginate fiber, a thermoplastic starch, or any combination thereof, 2) growing the cells to produce a composition comprising an extracellular matrix; and 3) tanning the composition comprising an extracellular matrix to form a cruelty-free leather. Disclosed herein in some embodiments, is a method of using a cruelty-free leather disclosed herein as a substitute for a traditional leather in a leather article. Disclosed herein is a leather article comprising a cruelty-free leather as disclosed herein. In some embodiments, a leather article can comprise a watch strap, a belt, a suspender, a packaging, a shoe, a boot, a footwear, a glove, a clothing, a bag, a clutch, a purse, a coin purse, a billfold, a key pouch, a credit card case, a pen case, a backpack, a case, a wallet, a saddle, a harness, a whip, a luggage, a travel good, a rucksack, a portfolio, a document bag, a briefcase, an attache case, a pet article, a leash, a collar, a hunting and fishing article, a gun case, a cutlery case, a holster for a fire arm, a stationary article, a writing pad, a book cover, a camera case, a spectacle case, a cigarette case, a cigar case, a jewel case, a mobile phone holster, a sport article, a ball, a basketball, a soccer ball, a football, or any combination thereof. In some embodiments, a clothing can comprise a top, a bottom, an outerwear, or any combination thereof. In some embodiments, a bag can comprise a handbag with or without shoulder strap. In some embodiments, a luggage can comprise a trunk, a suitcase, a travel bag, a beauty case, a toilet kit, or any combination thereof. Disclosed herein in some embodiments, is a method of using a composition as disclosed herein for a treatment of a disease or ailment. Disclosed herein in some embodiments, is a kit comprising a composition as disclosed herein or a leather article as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows microscope images of unstained fibroblast cells attached to a 1-ply nylon scaffold under rocking conditions. FIG. 1B shows a microscope image of unstained fibroblast cells attached to a 2-ply nylon scaffold under rocking conditions. FIG. 1C shows microscope an image of unstained fibroblast cells attached to a 40:60 bamboo/cotton scaffold under rocking conditions. FIG. 1D shows a microscope image of fibroblast cells attached to a 1-ply cotton scaffold material under rocking conditions. FIG. 1E shows microscope images of fibroblast cells stained with Calceim-AM attached to a 1-ply nylon scaffold under rocking conditions. FIG. 1F show microscope images of fibroblast cells stained with Calcein-AM attached to a 2-ply nylon scaffold under rocking conditions. FIG. 1G shows a microscope image of fibroblast cells stained with Calcein-AM attached to a 40:60 bamboo/cotton scaffold under rocking conditions. FIG. 1H shows a microscope image of fibroblast cells stained with Calcein-AM attached to a 1-ply cotton scaffold under rocking conditions. FIG. 1I shows a microscope image of fibroblast cells stained with Hoescht stain attached to a 1-ply nylon scaffold under rocking conditions. FIG. 1J shows a microscope image of fibroblast cells stained with Hoescht stain attached to a 2-ply nylon scaffold under rocking conditions. FIG. 1K shows a microscope image of fibroblast cells stained with Hoescht stain attached to a 40:60 bamboo/cotton blend scaffold material under rocking conditions. FIG. 1L shows a microscope image of fibroblast cells stained with a Hoescht stain attached to a 1-ply cotton scaffold under rocking conditions.

FIG. 2A shows scanning data of hides grown on light punch PLA scaffolds with a pore size of 55 μm. FIG. 2B shows scanning data of hides grown on PLA scaffolds with a pore size of 55 μm. FIG. 2C shows scanning data of hides grown on PLA scaffolds with a pore size of 80 μm. FIG. 2D shows hides grown on PLA scaffolds with a pore size of 100 μm.

FIG. 3A shows a histological image of a 5 μm cross-section of tissue stained using a trichrome stain where the scaffold is PLA with an average pore size of about 100 μm. FIG. 3B shows a histological image of a 5 μm cross-section of tissue stained using a trichrome stain where the scaffold is PLA with an average pore size of about 50 μm.

FIG. 4A shows a schematic of the experiment conducted to determine the impact of PLA scaffold soaking in FBS on cell attachment and proliferation. FIG. 4B shows cells/biopsy for samples grown on a PET control not soaked in serum as compared to PLA soaked and not soaked in serum.

FIG. 5 shows the percentage of cells on the PLA and Lyocell scaffolds which were unmodified, conjugated with pea protein, and conjugated with wheat protein determined by an MTT assay after 1 and 4 days where NT=non-treated, Pea=pea protein, and Wheat=wheat protein.

FIG. 6A shows a fluorescence microscopy image of fibroblast cells after 1 day growing on an unmodified PLA scaffold. FIG. 6B shows a fluorescence microscopy image of fibroblast cells after 1 day growing on a pea protein conjugated PLA scaffold. FIG. 6C shows a fluorescence microscopy image of fibroblast cells after 1 day growing on a wheat protein conjugated PLA scaffold. FIG. 6D shows a fluorescence microscopy image of fibroblast cells after 1 day growing on an unmodified Lyocell scaffold. FIG. 6E shows a fluorescence microscopy image of fibroblast cells after 1 day growing on a pea protein conjugated Lyocell scaffold. FIG. 6F shows a fluorescence microscopy image of fibroblast cells after 1 day growing on a wheat protein conjugated Lyocell scaffold.

FIG. 7A and FIG. 7B show histological images of 5 μm cross-sections of tissues stained with trichrome stain on a scaffold with labels highlighting dense tissue, fibers, nuclei, and less dense tissue.

FIG. 8A shows a histological image of a 5 μm cross-section of native bovine hide stained with a trichrome stain with labels indicating the location of the grain and the corium. FIG. 8B shows a histological image of a 5 μm cross-section of the top layer (grain) of a bovine hide stained with a trichrome stain.

FIG. 9A shows a fluorescence microscopy image of fibroblast cells at 4× zoom after the addition of Calcein AM. The live cells fluoresce green and arrows highlight the spreading of cells indicating affinity for the scaffold. FIG. 9B shows a fluorescence microscopy image of fibroblast cells at 10× zoom after the addition of Calcein AM. The live cells fluoresce green and arrows highlight the spreading of cells indicating affinity for the scaffold. A second arrow highlights the fiber illuminated by white light.

FIG. 10A shows a fluorescence microscopy image of fibroblast cells at 10× zoom after the addition of Calcein AM, causing the live cells to fluorescence green. The cells spreading and attaching highlights good affinity for the scaffold. FIG. 10B shows a fluorescence microscopy image of fibroblast cells at 10× zoom after the addition of Calcein AM, causing the live cells to fluoresce green. The cells rounding up and aggregating is indicative of poor affinity for the scaffold.

FIG. 11 shows a histological image of a 5 μm cross-section of tissue stained with a trichrome stain with labels indicating the slide overview, scale bar, cursor position, and tissue thickness.

FIG. 12 shows the remaining glucose levels in fresh media, 40:60 bamboo/cotton, 1-ply nylon, 2-ply nylon, and 1-ply cotton scaffolds in static and rocking conditions as measured from the last 4 feeds from day 10 to day 17 of the tissue culture.

FIG. 13 shows the basic physical data of tissues on 100 gsm (blue), 50 gsm (red), 55 μm pore size light punch (55LP) (green), pressed PET (PPET) (purple), and VL1 on PPET scaffolds (blue). The physical data includes harvest thickness, crust thickness, harvest weight, raw material weight, crust weight, and calculated tissue only weight.

FIG. 14A and FIG. 14B show histological images of a 5 μm cross-section of tissue grown on a 55 μm pore size light punch scaffold stained with a trichrome stain.

FIG. 15A, FIG. 15B, FIG. 15C, FIG. 15D, FIG. 15E, and FIG. 15F show histological images of 5 μm cross-sections of tissues grown on 50 gsm scaffolds stained with a trichrome stain.

FIG. 16A, FIG. 16B, FIG. 16C, FIG. 16D, FIG. 16E, and FIG. 16F show histological images of 5 μm cross-sections of tissues grown on 100 gsm scaffolds stained with a trichrome stain.

FIG. 17A shows a fluorescence microscopy image of fibroblast cells growing on an unmodified PLA scaffold after 4 days. FIG. 17B shows a fluorescence microscopy image of fibroblast cells growing on a pea protein conjugated PLA scaffold after 4 days. FIG. 17C shows a fluorescence microscopy image of fibroblast cells growing on a wheat protein conjugated PLA scaffold after 4 days. FIG. 17D shows fibroblast cells growing on an unmodified Lyocell scaffold after 4 days. FIG. 17E shows fibroblast cells growing on a pea protein conjugated Lyocell scaffold after 4 days. FIG. 17F shows fibroblast cells growing on a wheat protein conjugated Lyocell scaffold after 4 days.

FIG. 18 shows the cell viability of fibroblast growing on unmodified Lyocell (Lyo NT), Lyocell modified with pea protein (Lyo Pea) or wheat protein (Lyo Wheat), and PLA modified with pea protein (PLA Pea) or wheat protein (PLA Wheat) normalized to the viability of fibroblasts growing on unmodified PLA (PLA NT) on day 1 (blue) and day 4 (red).

FIG. 19 shows relative cell viability with respect to fibroblasts growing on unmodified PLA for fibroblasts growing on unmodified Lyocell (Lyo), pea protein conjugated Lyocell by cold adsorption (Lyo pea cold), pea protein conjugated Lyocell by a single autoclave cycle (Lyo pea auto 1), pea protein conjugated Lyocell by 2 autoclave cycles (Lyo pea auto 2), and pea protein conjugated Lyocell by 4 autoclave cycles (Lyo pea auto 4) after 1 day (blue) and 4 days (red).

FIG. 20A shows a fluorescence microscopy image after one day of fibroblast cells growing on an unmodified PLA scaffold after 4 days. FIG. 20B shows a fluorescence microscopy image of fibroblast cells growing on an unmodified Lyocell scaffold after 4 days. FIG. 20C shows a fluorescence microscopy image of fibroblast cells growing on a pea protein conjugated Lyocell scaffold wherein the pea protein was conjugated via autoclave after 4 days. FIG. 20D shows a fluorescence microscopy image of fibroblast cells growing on a pea protein conjugated PLA scaffold after 4 days. FIG. 20E shows a fluorescence microscopy image of fibroblast cells growing on an oxidized Lyocell scaffold after 4 days. FIG. 20F shows a fluorescence microscopy image of fibroblast cells growing on an oxidized Lyocell scaffold that has been functionalized with pea protein after 4 days.

FIG. 21 shows the relative cell viability of fibroblast cells with respect to cells growing on unmodified PLA for fibroblasts growing on pea protein conjugated PLA (PLA Pea), pea protein conjugated Lyocell (Lyocell Pea autoclaved), oxidized Lyocell conjugated with pea protein (Lyocell Pea oxidized), oxidized Lyocell, and unmodified Lyocell scaffolds after day 1 (blue) and day 4 (red).

FIG. 22 shows the average collagen concentration from tissue biopsies resulting from seeding densities of 125 k cells/cm², 250 k cells/cm², 500 k cells/cm², and 1M cells/cm² after 24 hours or 96 hours of digestion.

FIG. 23 shows the collagen concentration (wet weight) for tissues resulting from seeding densities of 62.5 k cells/cm², 125 k cells/cm², 500 k cells/cm² after 24 hours (blue) or 96 hours (orange) of digestion.

FIG. 24 shows the collagen concentration per unit wet weight in tissues resulting from seeding densities of 500 k cells/cm², 125 k cells/cm², 60 k cells/cm², and 30 k cells/cm² after digestion times of 24 hours (orange) and 96 hours (blue).

FIG. 25A shows a fluorescence microscopy image of fibroblast cells successfully attaching to a scaffold and spreading out. FIG. 25B shows a fluorescence microscopy image of fibroblasts unsuccessfully attaching to a scaffold and aggregating.

FIG. 26 shows the glucose concentration (g/L) after day 1 (blue), day 3 (red), and day 7 (green) of cell cultures growing on a control scaffold as well as a nylon, PLA, 50:50 PLA:Bioc PLA, cotton, trilobal viscose, regular viscose, and Lyocell scaffold materials.

FIG. 27 shows the collagen content normalized to the protein content (blue) and the biopsy (red) for tissues grown on a PET scaffold in hPL media and PLA, PVOH, PET, Lyocell with nylon mesh, 3.3 dtex Lyocell, 1.7 dtex Lyocell, 6.7 dtex Lyocell, and Lyocell with alginate scaffolds in FBS media.

FIG. 28 shows tanned hides resulting from tissue growth various scaffolds. In the top row the tissues are, from left to right, hPL PET, FBS PET, PVOH (Not Present), PLA in the top row and 1.7 dtex Lyocell, 3.3 dtex Lyocell, 6.7 dtex Lyocell, Alginate, and Nylon Mesh in the bottom row.

FIG. 29 shows a control hide, not exposed to high temperatures (left), a hide exposed to 95° C. to remove the scaffold after tanning (middle), and a hide exposed to 95° C. to remove the scaffold before tanning (right).

FIG. 30A shows a hide pre-tan (left) and post-tan (right) after the hide was treated with benzylamine. FIG. 30B shows a hide pre-tan (left) and post-tan (right) after the hide was treated with ethyl acetate. FIG. 30C shows a hide that was untreated with benzylamine or ethyl acetate.

FIG. 31 shows the collagen concentration (blue) and total protein concentration (red) for biopsied tissues grown on a 55 μm pore size scaffold (55-1), a 55 μm pore size light punch (55-LP-1) scaffold, an 80 μm pore size scaffold (80-1), and a 100 μm pore size scaffold (100-1).

FIG. 32 shows the collagen content after digestion for 24 hours (blue) or 96 hours (red) normalized by volume from tissues grown on a pressed control (Pressed PET), thinsulate, secant towel, secant spacer, fibertex, a double stack of pressed PET, smooth facing laminate, rough facing laminate, autoclaved laminate, and adsorbable only scaffold materials.

FIG. 33 shows potential normalizing factors including DNA (μg), thickness (mm*5), and weight (mg*10) for tissues grown on a pressed control (Pressed PET), thinsulate, secant towel, secant spacer, fibertex, a double stack of pressed PET, smooth facing laminate, rough facing laminate, autoclaved laminate, and adsorbable only scaffold materials.

FIG. 34 shows the thickness, weight, hydroxyproline content, and DNA content from tissues grown on a pressed control (PET) (blue) and fibertex (red).

FIG. 35 shows the collagen content after 24 hours or 96 hours of digestion of the biopsy after 4 weeks of growth or 8 weeks of growth for tissues grown on a pressed control (PET) (blue) and fibertex (red).

FIG. 36 shows a histological image of a 5 μm cross-section of tissue grown on a pressed PET scaffold stained with a trichrome stain.

FIG. 37 shows a histological image of a 5 μm cross-section of tissue grown on a double layer pressed PET scaffold stained with a trichrome stain.

FIG. 38 shows a histological image of a 5 μm cross-section of tissue grown on knitted towel like material stained with a trichrome stain.

FIG. 39A and FIG. 39B show histological images of 5 μm cross-sections of tissue exhibiting planar, aligned collagen arrangement, stained with a trichrome stain.

FIG. 40A and FIG. 40B show histological images of 5 μm cross-sections of tissue grown on knitted towel-like material stained with a trichrome stain.

FIG. 41A and FIG. 41B show images of a knitted towel like material in which tissues can be grown on.

FIG. 42 shows a histological image of a 5 μm cross-section of tissue grown on a vicryl and pressed laminate scaffold stained with a trichrome stain.

FIG. 43 shows a histological image of a 5 μm cross-section of tissue grown on a vicryl and rough pressed laminate scaffold stained with a trichrome stain.

FIG. 44 shows a histological image of a 5 μm cross-section of tissue grown on a vicryl and autoclaved pressed laminate scaffold stained with a trichrome stain.

FIG. 45 shows a histological image of a 5 μm cross-section of tissue grown on a fibertex scaffold stained with a trichrome stain.

FIG. 46 shows a histological image of a 5 μm cross-section of tissue grown on a thinsulate scaffold stained with a trichrome stain.

FIG. 47 shows a histological image of a 5 μm cross-section of tissue grown on a vicryl only scaffold stained with a trichrome stain.

### DETAILED DESCRIPTION

Several aspects are described below with reference to example applications for illustration. It should be understood that numerous specific details, relationships, and methods are set forth to provide a full understanding of the features described herein. Features described herein can be practiced without one or more of the specific details or with other methods. The features described herein are not limited by an illustrated ordering of acts or events, as some acts can occur in different orders and/or concurrently with other acts or events. Furthermore, not all illustrated acts or events may be required to implement a methodology in accordance with the features described herein.

The terminology used herein can be for the purpose of describing particular cases only and is not intended to be limiting. As used herein, the singular forms “a”, “an” and “the” are intended to include the plural forms as well, unless the context clearly indicates otherwise. Furthermore, to an extent that the terms “including”, “includes”, “having”, “has”, “with”, or variants thereof are used in either the detailed description and/or the claims, such terms are intended to be inclusive in a manner similar to the term “comprising”.

In this disclosure the term “about” or “approximately” can mean a range of up to 10% of a given value. In this disclosure the term “substantially” refers to something that can be done to a great extent or degree.

As used herein the term “fibroblast cell” can include a connective tissue cell that is found in the skin and tendons of a body. A fibroblast cell can be obtained from a biopsy of a skin or tendon. Fibroblast cells are also ubiquitous in many tissues and organs besides skin and tendons. A fibroblast cell can be a type of biological cell that synthesizes extracellular matrix and collagen. A fibroblast cell can be a type of biological cell that produces the structural framework (stroma) for animal tissues and plays a critical role in wound healing. Fibroblasts can be the most common cells of connective tissue in animals. A fibroblast cell can comprise a major part of a tissue layer grown in culture, which can be tanned into a leather.

As used herein the term “fibroblast-like cell” can refer to a fibroblast cell that has been grown in culture, has been immortalized, or a combination thereof. A fibroblast-like cell can comprise a cell that has been differentiated to have a morphology, phenotype, or combination thereof that substantially resembles a fibroblast cell. A fibroblast-like cell can comprise genetic or phenotypic alterations from a fibroblast cell, while still expressing substantially similar gene expression to a fibroblast cell. A fibroblast-like cell can have similar characteristics to a fibroblast cell such as the ability to synthesize collagen, extracellular matrix, the structural matrix (stroma) of animal tissue, or any combination thereof. A fibroblast-like cell can produce substantially similar levels of collagen compared to a fibroblast cell.

As used herein, the term “pluripotent stem cell” can refer to any precursor cell that has an ability to form any adult cell other than placenta.

As used herein, the term “embryonic stem cells” or “ES cells” or “ESC” can refer to precursor cells that have an ability to form any adult cell.

As used herein, the term “induced pluripotent stem cells” or “iPS cells” or “iPSCs” can refer to a type of pluripotent stem cell artificially derived from a non-pluripotent cell (e.g., an adult somatic cell). Induced pluripotent stem cells can be identical to embryonic stem cells in an ability to form any adult cell but may not be derived from an embryo.

As used herein the term “isolated” can refer to a cell that has been removed from an animal or human body. An isolated cell can be grown in culture or in vitro. An isolated cell can be in contact with other isolated cells, a scaffold, a medium, or a combination thereof.

As used herein, the terms “decellularize” or “decellularized” can refer to the removal of cells from a cell layer. The term “at least partially decellularized” can refer to the removal of at least some cells from a cell layer. “Decellularization” can refer to the process of removing cells to make a decellularized cell layer, and can be achieved through methods such as salting, or the use of detergents.

As used herein, the term “cruelty-free leather” can refer to a leather material described herein that can serve as a leather material for any mammal or non-mammal. The disclosure herein can be practiced with human and non-human mammals, such as non-human primates and members of the bovine, ovine, porcine, equine, canine and feline species as well as rodents such as mice, rats and guinea pigs, members of the lagomorph family including rabbit, fish including shark and stingray, birds including ostrich and reptiles including lizards, snakes and crocodiles. In some embodiments, a cruelty-free leather can comprise an artificial leather. In some embodiments, a cruelty-free leather can comprise an eco-friendly leather, a plastic free leather, or any combination thereof. In some embodiments, a cruelty-free leather can comprise a tanned artificial cell layer, a tanned at least partially decellularized cell layer, or a combination thereof. In some embodiments, a cell layer or an at least partially decellularized cell layer can comprise a dermal layer, an epidermal layer, an at least partially decellularized dermal layer, an at least partially decellularized epidermal layer, or any combination thereof. In some embodiments, a mammalian cruelty-free leather which can be formed can be dependent on a source of a cell used in an invention described herein, e.g., keratinocytes and fibroblasts, e.g., when bovine keratinocytes and fibroblasts can be used to form a skin equivalent, a bovine cruelty-free leather can be formed.

### Cruelty-Free Leather Compositions

Disclosed herein in some embodiments are compositions comprising scaffolds for culturing cells. In some embodiments, a scaffold can provide a substrate or support for a cell to attach, to grow on, to generate an extracellular matrix, or any combination thereof. In some embodiments, a scaffold and cell composition disclosed herein can be tanned to produce a cruelty-free leather. In some embodiments, a method or composition disclosed herein can be used to generate a skin equivalent. In some embodiments, a composition disclosed herein can be transplanted onto a patient in need thereof for the treatment of a disease or ailment. In some embodiments, a cell, a cell layer, a layered structure, or a cruelty-free leather can be seeded onto a scaffold. In some embodiments, a scaffold can comprise a substrate. In some embodiments, a scaffold can provide a certain firmness (e.g., resistance to tearing), elasticity, or both. In some embodiments, a cruelty-free leather can comprise a part of or an entire scaffold. In some embodiments, a cruelty-free leather may not comprise a scaffold. In some embodiments, after assisting a formation of a layer in a cruelty-free leather, a scaffold can be removed from a final cruelty-free leather product. In some embodiments, a scaffold comprised in a cruelty-free leather may be degraded after a period of time. In some embodiments, a scaffold can be degradable, biodegradable, bioabsorbable, resorbable, or any combination thereof.

In some embodiments, a scaffold can comprise a three-dimensional woven material. In some embodiments, a three-dimensional woven material can comprise a spacer fabric. In some embodiments, a spacer fabric can comprise a plurality of faces. In some embodiments, a plurality of faces can comprise a front face and a back face. In some embodiments, a face can comprise a woven material. In some embodiments a filler can separate a front face from a back face. In some embodiments, a filler can keep a front face at a distance from a back face. In some embodiments, a filler can provide a gap to allow nutrient entry, waste removal, cell growth, or any combination thereof.

In some embodiments, a scaffold can be made of natural materials, synthetic materials, or any combination thereof. In some embodiments, a scaffold can comprise a substrate. In some embodiments, a substrate can comprise a substrate for cell growth. In some embodiments, a scaffold can be formed using a net made of a bioabsorbable synthetic polymer. In some embodiments, a scaffold can be formed by attaching a nylon net to a silicon film. In some embodiments, a scaffold can comprise a two-layered structure of a collagen sponge and a silicon sheet. In some embodiments, a scaffold can be formed using an atelocollagen sponge. In some embodiments, a scaffold can be made into a sheet. In some embodiments, a scaffold can be formed by matching collagen sponges having different pore sizes. In some embodiments, acellular dermal matrices (ADM) can be formed using fibrin glue, allogeneic skin, or a combination thereof that has been at least partially decellularized.

### Scaffold Materials

Disclosed herein in some embodiments, is a composition comprising a scaffold in contact with an extracellular matrix comprising a collagen, wherein a scaffold can comprise a non-woven, needle-punched material. Disclosed herein in some embodiments, is a composition comprising a scaffold in contact with an extracellular matrix comprising a collagen, wherein a scaffold can comprise a three-dimensional woven material. Disclosed herein in some embodiments, is a composition comprising a scaffold in contact with an extracellular matrix comprising a collagen, wherein a scaffold can comprise a polycaprolactone (PCL), a polylactic acid (PLA), a polylactic and polyglycolic acid (PLGA), a polyethylene terephthalate (PET), a nylon, a polyethylene (PE), a polyethylene furanoate (PEF), a polypropylene (PP), a polyvinyl alcohol (PVA), a cotton, a bast fiber, a viscose, a modal, a lyocell, a plant based protein fiber, a biobased material, a cellulose, an alginate fiber, a thermoplastic starch, or any combination thereof. In some embodiments, a scaffold can comprise a polycaprolactone. In some embodiments, a scaffold can comprise a polylactic acid. In some embodiments, a scaffold can comprise a polylactic and polyglycolic acid. In some embodiments, a scaffold can comprise a polyethylene terephthalate. In some embodiments, a scaffold can comprise a nylon. In some embodiments, a scaffold can comprise a polyethylene. In some embodiments, a scaffold can comprise a polyethylene furanoate. In some embodiments, a scaffold can comprise a polypropylene. In some embodiments, a scaffold can comprise a polyvinyl alcohol. In some embodiments, a scaffold can comprise a cotton. In some embodiments, a scaffold can comprise a bast fiber. In some embodiments, a scaffold can comprise a viscose. In some embodiments, a scaffold can comprise a modal. In some embodiments, a scaffold can comprise a lyocell. In some embodiments, a scaffold can comprise a plant-based protein fiber. In some embodiments, a scaffold can comprise a biobased material. In some embodiments, a scaffold can comprise a cellulose. In some embodiments, a scaffold can comprise an alginate fiber. In some embodiments, a scaffold can comprise a thermoplastic starch.

The methods and compositions provided herein may provide design advantages over the use of natural leather. In some embodiments, the selection of a scaffold material, such as described in Example 13, may have an effect on the physical properties of the resulting material. In some embodiments, the selection of the scaffold material may have an effect on the tear strength (e.g., average double tear strength) of the resulting material. In some embodiments, the selection of the scaffold material may result in a stronger material. In some embodiments, the selection of the scaffold material may result in a weaker material. In some embodiments, as described in Example 13, the compositions provided herein may comprise similar (e.g., tear) strengths to natural (e.g., cow) hides, while being thinner than natural hides.

In some embodiments, a scaffold can comprise natural substances such as collagen (e.g., collagen matrix), natural adhesive (e.g., fibrin glue, cold glues, animal glue, blood albumen glue, casein glue, or vegetable glues such as starch and dextrin glues). In some embodiments, a scaffold can comprise a polylactide, a polyglycolide, a polycaprolactone, a hydrogel, or any combination thereof. In some embodiments, a scaffold can comprise a silk. In some embodiments, a scaffold can be made of a silk. In some embodiments, a scaffold can comprise a silk fibroin, a cellulose, a cotton, an acetate, an acrylic, a latex fiber, a linen, a nylon, a rayon, a velvet, a modacrylic, an olefin polyester, a saran, a vinyon, a wool, a jute, a hemp, a bamboo, a flax or a combination thereof. In some embodiments, a scaffold can comprise a fiber. In some embodiments, a fiber can be a fiber of a silk, a cotton, a wool, a wood, a cellulose extracted in particular from a wood, a vegetable, an algae, a polyamide, a modified cellulose, a poly-p-phenyleneterephthalamide, an acrylic fiber, for example those of polymethyl methacrylate or of poly-2-hydroxyethyl methacrylate, fibers of polyolefin for example fibers of polyethylene or polypropylene, glass, silica, aramid, carbon, for example in a form of a graphite, a poly(tetrafluoroethylene), an insoluble collagen, a polyester, a polyvinyl chloride, a polyvinylidene chloride, a polyvinyl alcohol, a polyacrylonitrile, a chitosan, a polyurethane, a poly(urethane-urea), a polyethylene phthalate, and fibers formed from a blend of polymers such as those mentioned above, such as polyamide/polyester fibers, or any combination thereof. In some embodiments, a scaffold comprises cellulose or a modified cellulose. In some embodiments, a modified cellulose can comprise a rayon, a viscose, an acetate, a rayon acetate, or any combination thereof.

In some embodiments, such as described in Example 1, the scaffold is a bamboo/cotton blend, 1-ply nylon, 2-ply nylon, or 1-ply nylon. In some embodiments, such as described in Example 1, high levels of growth and spindle-like cells are achieved using bamboo/cotton scaffolds.

In some embodiments, a scaffold can comprise a polymer. In some embodiments, a polymer can comprise a biopolymer. In some embodiments, a biopolymer can include but may not be limited to a chitin, a chitosan, an elastin, a collagen, a keratin or a polyhydroxyalkanoate. In some embodiments, a polymer can be biodegradable, biostable, or a combination thereof. In some embodiments, a polymer in a scaffold can be a natural polymer. In some embodiments, an exemplary natural polymer can include a polysaccharide such as an alginate, a cellulose, a dextran, a pullane, a polyhyaluronic acid, a chitin, a poly(3-hydroxyalkanoate), a poly(3-hydroxyoctanoate), a poly(3-hydroxyfatty acid), or any combination thereof. In some embodiments, a polymer can comprise a polyethylene (PE), a polypropylene (PP), a Polyethylene terephthalate (PET), a Polyamide 6,6 (PA 6,6), a Polyamide 11 (PA 11), a Polyvinylidene fluoride (PVDF), a Polyethylene furanoate (PEF), a Polyurethane (PU), a Polyhydroxyalkanoate (PHA), a Polyhydroxybutyrate (PHB), a Poly(3-hydroxybutyrate-co-3-hydroxyvalerate) (PHBV), a Polylactic acid (PLA), a Polycaprolactone (PCL), a Polybutylene succinate (PBS), a Poly(glycolic) acid (PGA), a Poly(lactic-co-glycolic acid (PLGA), a Polyvinyl Alcohol (PVOH), an Alginate, a Copolymer PEGylated fibrin (P-fibrin), a Poly(glycerol sebacate) (PGS), a poly(L-lactic acid) (PLLA), a Poly(lactic-coglycolic acid) (PLGA), a Poly-D,L-lactic acid/polyethylene glycol/poly-D,L-lactic acid (PDLLA-PEG), a hyaluronic acid (HA), or any combination thereof. In some embodiments, a scaffold can also comprise a chemical derivative of a natural polymer. In some embodiments, a chemical derivative can include a substitution and/or an addition of a chemical group such as an alkyl, an alkylene, a hydroxylation, an oxidation, another chemical modification, or any combination thereof. In some embodiments, a natural polymer can also be selected from a protein such as collagen, zein, casein, gelatin, gluten, and serum albumen. In some embodiments, a polymer in a scaffold can be biodegradable synthetic polymers, including poly alpha-hydroxy acids such as poly L-lactic acid (PLA), polyglycolic acid (PGA) or copolymers thereof (e.g., poly D,L-lactic co-glycolic acid (PLGA)), and hyaluronic acid.

In some embodiments, a scaffold can comprise a polyvinyl alcohol (PVA), polyvinyl acetate (PVA), polycaprolactone (PCL), a polylactic acid (PLA), a polylactic and polyglycolic acid (PLGA), a polyethylene terephthalate (PET), a nylon, a polyethylene (PE), a polyethylene furanoate (PEF), a polypropylene (PP), a polyvinyl alcohol (PVA), polyhydroxyalkanoate (PHA), polyhydroxybutyrate (PHB), polycaprolactone (PCL), alginate, oxidized alginate, a cotton, a bast fiber, a viscose, a modal, a lyocell, a plant based protein fiber, a biobased material, a viscose, a cellulose, an alginate fiber, a thermoplastic starch, or any combination thereof. In some embodiments, the scaffold comprises polyhydroxyalkanoate (PHA). In some embodiments, the scaffold comprises polyhydroxybutyrate (PHB). In some embodiments, the scaffold comprises polycaprolactone (PCL). In some embodiments, the scaffold comprises oxidized alginate. In some embodiments, the scaffold comprises polyvinyl alcohol (PVOH). In some embodiments, the scaffold comprises polyvinyl acetate (PVA).

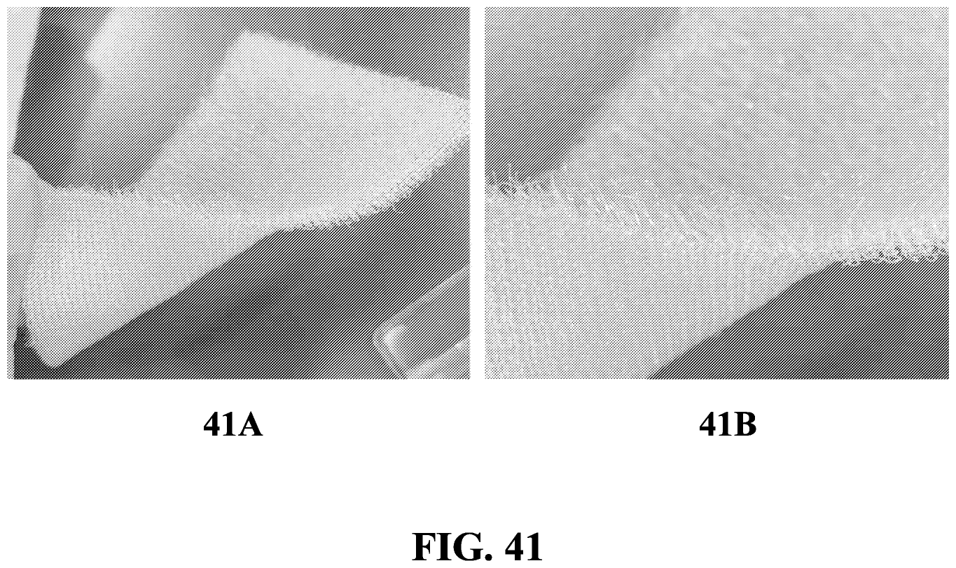

In some embodiments, such as described in Example 11, a scaffold can comprise 3M Thinsulate material Type G, comprising 3 main layers, 2 spun-bond, and one air-laid nonwoven, which are sandwiched. In some embodiments, a scaffold can comprise a towel-like material made of PET (FIG. 41A-B). In some embodiments, a scaffold can comprise two fabrics held apart by fibers running between two spacing the two outer fabrics by a set distance. In some embodiments, a scaffold can comprise a single fiber population of lyocell fibers (e.g., “Fibertex”). In some embodiments, a scaffold can comprise a needlepunch nonwoven material, pressed on one side, and laminated to a second by sutures. In the aforementioned material, the orientation of the two needlepunch nonwoven materials can be such that the pressed sides of each of the two materials can be facing outward or inward. In some instances, the needlepunch nonwoven materials can be autoclaved prior to assembly. In some embodiments, a scaffold can comprise one or more vicryl meshes attached together. In some instances, as described in Example 11, different scaffolds described herein can be used to achieve different tissue characteristics.

In some embodiments, a scaffold can be bioabsorbable. In some embodiments, a bioabsorbable scaffold can be a non-cytotoxic structure or substance that may be capable of containing or supporting living cells and holding them in a desired configuration for a period of time. In some embodiments, the term “bioabsorbable” can refer to any material a body can break down into non-toxic by-products that can be excreted from a body or metabolized therein. In some embodiments, an exemplary bioabsorbable material for a scaffold can include, a poly(lactic acid), a poly(glycolic acid), a poly(trimethylene carbonate), a poly(dimethyltrimethylene carbonate), a poly(amino acids)s, a tyrosine-derived poly(carbonates)s, a poly(carbonates)s, a poly(caprolactone), a poly(para-dioxanone), a poly(esters)s, a poly(ester-amides)s, a poly(anhydrides)s, a poly(ortho esters)s, a collagen, a gelatin, a serum albumin, a protein, a polysaccharide, a mucopolysaccharide, a carbohydrate, a glycosaminoglycan, a poly(ethylene glycols), a poly(propylene glycols), a poly(acrylate esters), a poly(methacrylate esters), a poly(vinyl alcohol), a hyaluronic acid, a chondroitin sulfate, a heparin, a dermatan sulfate, a versican, a copolymer, a blend of polymers, a mixture of polymers, an oligomer containing bioabsorbable linkages, or any combination thereof.

In some embodiments, a scaffold can be a mesh. In some embodiments, a mesh can be a network of material (e.g., threads, strings, strands, fibers, or any combination thereof) that can be connected by weaving or otherwise. In some embodiments, a mesh can comprise a material that can be artificial, biological or any combination thereof. In some embodiments, a mesh can have pores that can be regular or irregular in size, regular or irregular in shape, regular or irregular in pattern, or any combination thereof. In some embodiments, a mesh can be two-dimensional or three-dimensional. In some embodiments, a mesh can have a pore from about: 10 nm to 10 cm in diameter, spacing, or any combination thereof. In some embodiments, a pore size can be generally about: 10 nm, 20 nm, 50 nm, 100 nm, 200 nm, 300 nm, 400 nm, 500 nm, 600 nm, 700 nm, 800 nm, 900 nm, 1 mm, 1.1 mm, 1.2 mm, 1.3 mm, 1.4 mm, 1.5 mm, 1.6 mm, 1.7 mm, 1.8 mm, 1.9 mm, 2 mm, 3 mm, 4 mm, 5 mm, 6 mm, 7 mm, 8 mm, 9 mm, 10 mm, 12 mm, 15 mm, 2 cm, 2.5 cm, 3 cm, 4 cm, 5 cm, 6 cm, 7 cm, 8 cm, 9 cm, or 10 cm. In some embodiments, a pore size can be at least 10 nm (e.g., 50 nm, 100 nm, 500 nm, 1 mm). In some embodiments, a pore size can be at most 1 cm (e.g., 5 cm, 10 mm, 5 mm, 1 mm). A mesh can have a diameter of about: 20 nm, 50 nm, 100 nm, 200 nm, 300 nm, 400 nm, 500 nm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 3 mm, 4 mm, 5 mm, 6 mm, 7 mm, 8 mm, 9 mm, 10 mm, 12 mm, 15 mm, 2 cm, 2.5 cm, 3 cm, 4 cm, 5 cm, 6 cm, 7 cm, 8 cm, 9 cm, or 10 cm. In some embodiments, a mesh can have a diameter of at least 20 nm (e.g., 50 nm, 100 nm, 500 nm, 1 mm). In some embodiments, a mesh can have a diameter of at most 3 cm (e.g., 1 cm, 500 mm, 100 mm, 10 mm, 1 mm). In some embodiments, a strand (e.g., fiber, threads, webs, etc.) or material forming a mesh can have diameters of from about 50 nm to about 10 mm in diameter. In some embodiments, a mesh may not be a scaffold.

In some embodiments, a scaffold can be a supporting structure for cell proliferation. In some embodiments, a scaffold can be permeable to a fluid, a nutrient, such that a cell culture medium can contact a surface of a cell layer.

In some embodiments, a scaffold can comprise a textile comprised of entangled fibers. In some embodiments, fibers can be entangled by means of a woven, knitted, or non-woven textile fabrication technology or some combination thereof. In some embodiments, a scaffold can be porous, biocompatible, sterilizable, mechanically and chemically stable, consistent and any combination thereof. In some embodiments, a scaffold can comprise a three-dimensional structure. In some embodiments, a cell can be seeded within a scaffold. In some embodiments, a scaffold can be of various thicknesses. In some embodiments, a scaffold can have a thickness that may be suitable for forming a cell layer. In some embodiments, a scaffold can have a thickness from about 0.1 mm to about 10 mm, such as from about 0.1 mm to about 5 mm, from about 0.1 mm to about 4 mm, from about 0.1 mm to about 3 mm, from about 0.1 mm to about 2 mm, to about 0.1 mm to about 1 mm, from about 0.2 mm to about 1 mm, from about 0.3 mm to about 1 mm, from about 0.4 mm to about 1 mm, from about 0.5 mm to about 1 mm, from 0.3 mm to about 1.5 mm, from about 0.4 mm to about 1.2 mm, from about 0.6 mm to about 1.2 mm, or from about 0.7 mm to about 1.5 mm. In some embodiments, a scaffold can have a thickness from about 0.5 mm to 1 mm. In some embodiments, a scaffold can be at least 0.1 mm, 0.2 mm, 0.3 mm, 0.4 mm, 0.5 mm, 0.8 mm, 1 mm, 2 mm, 3 mm, 4 mm, or 5 mm thick. In some embodiments, a scaffold can be at most 0.5 mm, 0.8 mm, 1 mm, 2 mm, 3 mm, 4 mm, 5 mm, 6 mm, 7 mm, 8 mm, 9 mm, or 10 mm thick. In some embodiments, a scaffold can have a length and/or a width of a cell layer to be placed and/or grown upon a scaffold. In some embodiments, a scaffold can have a length and/or a width of a cell layer described herein. In some embodiments, a scaffold can comprise a pore size of less than 1 nanometer. In some embodiments, a scaffold can comprise a pore size of greater than 1 nanometer. In some embodiments, a scaffold can comprise a pore size of between 10 μm and 900 μm.

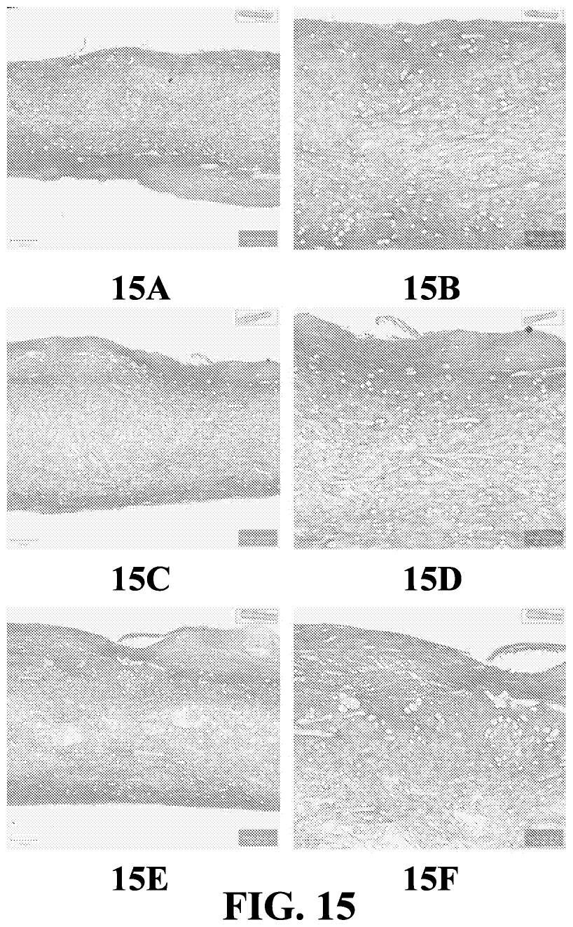

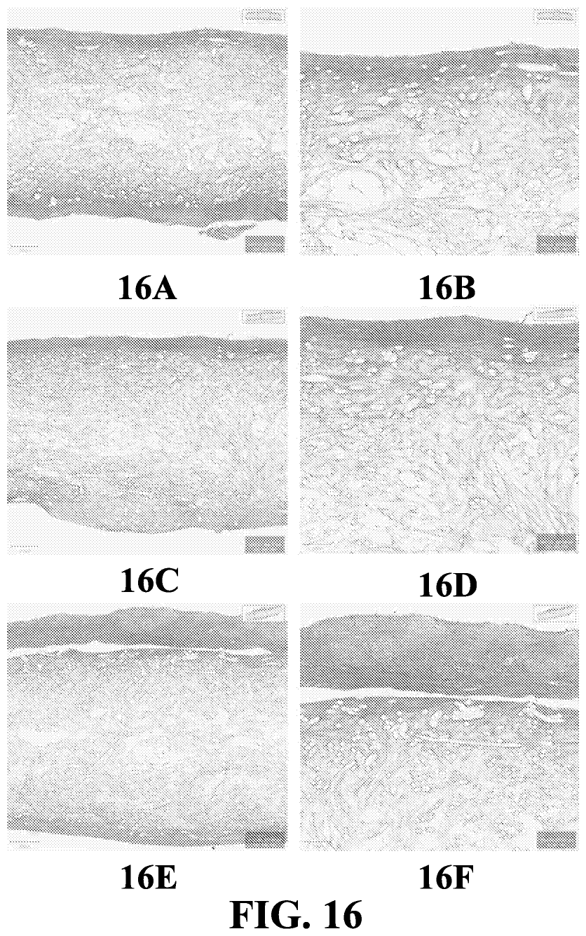

In some instances, such as described in Example 3, pore size may have an effect on tissue ingrowth. In some instances, larger pore sizes lead to better tissue in growth (FIG. 15A-F). In some instances, smaller pores lead to more weight contribution from the fibers creating the illusion of a weightier, stronger hide (FIG. 16A-F).

In some embodiments, a scaffold can comprise a three-dimensional woven material. In some embodiments, a three-dimensional woven material can comprise a spacer fabric. In some embodiments, a spacer fabric can comprise a front face in contact with a filler, and a back face in contact with a filler, wherein a filler separates a front face from a back face. In some embodiments, a separation of a front face from a back face by a filler can create a gap to allow nutrient entry, waste removal, cell attachment, cell growth, or any combination thereof. In some embodiments, a three-dimensional woven material can comprise a pile weave. In some embodiments, a three-dimensional woven material can comprise a terry, a frieze, a velvet, a corduroy, a velveteen, or any combination thereof. In some embodiments, a scaffold can comprise multiple layers of scaffold material. In some embodiments, multiple layers of scaffold material can comprise a combination of different form factors. In some embodiments, different form factors can comprise a non-woven material, a woven material, a needle-punched material, a three-dimensional structure, or any combination thereof. In some embodiments, a scaffold can comprise a three-layered composite material, wherein a three layered composite material can comprise two outer layers and one inner layer. In some embodiments, two outer layers can comprise a surface layer characteristic, and an inner layer can comprise a bulk property. In some embodiments, multiple layers can comprise multiple layers of a thin material. In some embodiments, multiple layers can be fused together, held together by entanglement, laminated together, stitched together, glued together, woven together, printed together, or any combination thereof.

In some embodiments, a non-woven, needle punched material can comprise a first plurality of pores. In some embodiments, a scaffold can comprise a non-woven configuration that can create a first plurality of pores. In some embodiments, a first plurality of pores can comprise an average pore size of about 30-70 μm. In some embodiments, a first plurality of pores can comprise an average pore size of about 80-120 μm. In some embodiments, a first plurality of pores can comprise an average pore size of about 50 μm or about 100 μm. In some embodiments, a scaffold can comprise a second plurality of pores produced by a needle punch.

In some embodiments, a scaffold can comprise a terry cloth. In some embodiments, a terry cloth can comprise an all in one textile that includes a high porosity section (fiber loops) and a stable backbone (a woven mesh base) that allow for two main structural needs of a scaffold: a high porosity environment for tissue growth and mechanical support for end product use mechanics.

In some embodiments, a scaffold can comprise a spacer fabric. In some embodiments, a spacer fabric can comprise both a stable backbone and a high porosity environment. In some embodiments, a center can be high porosity and high volume allowing for bulk tissue growth. In some embodiments, two separate surfaces can be different from each other, allowing for further specification of function. In some embodiments, a top surface can be highly porous and dissolvable so as to allow for a smooth top surface that is fiber free, while a bottom surface can be made out of a stable material to provide mechanical support.

In some embodiments, a cell layer may not form on a scaffold. In some embodiments, a dermal layer may not form on a scaffold (e.g., collagen matrix). In some embodiments, a cruelty-free leather does not comprise a scaffold. In some embodiments, a cell can form on a scaffold and then be substantially removed from a scaffold prior to tanning. In some embodiments, a scaffold can comprise a dissolvable scaffold, wherein a scaffold can be in contact with a solvent that can dissolve a scaffold.

In some embodiments, a solvent provided herein can be any suitable solvent to dissolve or otherwise contain a degradant required to degrade or otherwise dissolve the scaffold. In some embodiments, the solvent comprises water. In some embodiments, the solvent comprises an organic solvent. In some embodiments, the solvent comprises acetone, benzylamine, ethyl acetate, or a combination thereof. In some embodiments, the solvent comprises acetone. In some embodiments, the solvent comprises ethyl acetate. In other embodiments, the solvent comprises benzylamine. In some embodiments, the solvent comprises benzene, acetonitrile, ethanol, diethyl ether, methylene chloride, or tetrahydrofuran. In some embodiments, a temperature of the solvent is below the boiling point of the solvent. In some embodiments, a solvent can be at a temperature of at least 30° C. (e.g., at least 40° C., at least 50° C., at least 60° C., at least 70° C., at least 80° C., at least 90°, at least 100° C., or at least 110° C.). In some embodiments, a solvent can be a temperature of at most 200° C. (e.g., at most 180°, at most 160° C., at most 140° C., at most 120°, at most 100° C.). In some embodiments, a solvent can be at a temperature of about 40° C. to about 50° C. In some embodiments, a solvent can be at a temperature of 25° C. to about 125° C. In some embodiments, a solvent can be at a temperature of about 25° C. to about 100° C.

In some embodiments, a scaffold can comprise a thermoplastic polymer. In some embodiments, the thermoplastic polymer comprises polyvinyl acetate (PVA), polyvinyl alcohol (PVOH), or polylactic acid (PLA). In some embodiments, the thermoplastic polymer comprises polyvinyl acetate (PVA). In some embodiments, the thermoplastic polymer comprises polyvinyl alcohol (PVOH). In some embodiments, the thermoplastic polymer comprises polylactic acid (PLA). In some embodiments, the scaffold comprises a polyhydroxyalkanoate (PHA), polyhydroxybutyrate (PHB), polycaprolactone (PCL), or oxidized alginate. In some embodiments, the thermoplastic polymer comprises polyhydroxyalkanoate (PHA). In some embodiments, the thermoplastic polymer comprises polyhydroxybutyrate (PHB). In some embodiments, the thermoplastic polymer comprises polycaprolactone (PCL). In some embodiments, the thermoplastic polymer comprises oxidized alginate.

In some embodiments, a thermoplastic polymer comprises polyvinyl alcohol (PVA). In some embodiments, a solvent comprises water. In some embodiments, a thermoplastic polymer can comprise a polyvinyl alcohol (PVA), and wherein a solvent can comprise a water.

In some embodiments, a scaffold comprises a polylactic acid (PLA). In some embodiments, a solvent comprises a benzyl group, an ethyl group, a haloalkane, or a combination thereof. In some embodiments, a scaffold can comprise a polylactic acid (PLA), and wherein a solvent can comprise a benzyl group, an ethyl group, a haloalkane, or a combination thereof. In some embodiments, a solvent can comprise a benzylamine.

In some embodiments, a scaffold can comprise a biobased material, such as a biobased material provided elsewhere herein. In some embodiments, a solvent provided herein comprises a cellulase (e.g., such as a to degrade the biobased material). In some embodiments, a scaffold can comprise a biobased material and a solvent can comprise an enzyme that degrades a biobased material. In some embodiments, a scaffold can comprise a cellulose and a solvent can comprise a cellulase.

In some embodiments, removing a scaffold material from a cell and scaffold composition can comprise using a dissolution method. In some embodiments, a dissolution can comprise contacting a scaffold with a solvent. In some embodiments, a solvent can comprise a benzylamine, hot water, an enzyme, or any combination thereof. In other embodiments, a solvent comprises an organic solvent. In some embodiments, the organic solvent comprises acetone, benzylamine, or ethyl acetate. In some embodiments, a solvent can be applied to a scaffold at some stage of growth or post processing to remove a scaffold. In some embodiments, the temperature of the solvent varies during the dissolution method. In some embodiments, the temperature of the solvent is below the boiling point of the solvent. In some embodiments, the temperature of the solvent is no more than 150° C. (e.g., no more than 140° C., no more than 130° C., no more than 120° C., no more than 110° C., no more than 100°). In some embodiments, the temperature of the solvent is at least 30° C. (e.g., at least 40° C., at least 50° C., at least 60° C., at least 70° C., at least 80° C., at least 90°, at least 100° C., or at least 110° C.). In some embodiments, the temperature of the solvent is about 25° C. to about 100° C. In some embodiments, the temperature of the solvent is about 40° C. to about 50° C.

In some embodiments, removing a scaffold material from a cell and scaffold composition can comprise using a degradation method. In some embodiments, the scaffold is degraded over time through hydrolytic degradation. In some embodiments, the scaffold is degraded over time through hydrolytic degradation in neutral aqueous solution. In some embodiments, the scaffold comprises a polyhydroxyalkanoate (PHA), polyhydroxybutyrate (PHB), polycaprolactone (PCL), polylactic acid (PLA), or oxidized alginate. In some embodiments, the scaffold comprises a polyhydroxyalkanoate (PHA). In some embodiments, the scaffold comprise a polyhydroxybutyrate (PHB). In some embodiments, the scaffold comprises a polycaprolactone (PCL). In some embodiments, the scaffold comprises a polylactic acid (PLA). In some embodiments, the scaffold comprises oxidized alginate.

In some embodiments, a dissolution can comprise contacting a scaffold with a dissolution agent. In some embodiments, a solvent can comprise a dissolution agent. In some embodiments, a dissolution agent can comprise an enzyme. In some embodiments, a dissolution agent comprises a cellulase. In some embodiments, an enzyme can comprise cellulase. In some embodiments, an enzyme can comprise cellulase to digest cellulose. In some embodiments, a dissolution agent comprises a lipase. In some embodiments, the dissolution agent comprises an alginate lyase. In some embodiments, the scaffold comprises a calcium alginate and the dissolution agent comprises an alginate lyase. In other embodiments, the scaffold comprises an ester-containing polymer and the dissolution agent comprises an esterase. In some embodiments, the scaffold comprises an ester-containing polymer and the dissolution agent comprises a lipase.

In some embodiments, a dissolution agent comprises a chelate. In some embodiments, the dissolution agent comprises ethylenediaminetetraacetic acid (EDTA). In some embodiments, the scaffold comprises an alginate and the dissolution agent comprises ethylenediaminetetraacetic acid (EDTA).

In some embodiments, the dissolution agent comprises a corrosive agent. In some embodiments, the dissolution agent comprises a strong acid. In some embodiments, the strong acid comprises hydrochloric acid, nitric acid, hydroiodic acid, perchloric acid, chloric acid, or a combination thereof. In some embodiments, the strong acid comprises hydrochloric acid. In some embodiments, the strong acid comprises nitric acid. In some embodiments, the strong acid comprises perchloric acid. In some embodiments, the strong acid comprises hydroiodic acid. In some embodiments, the strong acid comprises chloric acid. In other embodiments, the dissolution agent comprises a strong base. In some embodiments, the strong base comprises lithium hydroxide, sodium hydroxide, potassium hydroxide, calcium hydroxide, strontium hydroxide, barium hydroxide, or a combination thereof. In some embodiments, the strong base comprises sodium hydroxide. In some embodiments, the strong base comprises lithium hydroxide. In some embodiments, the strong base comprises potassium hydroxide. In some embodiments, the strong base comprises calcium hydroxide. In some embodiments, the strong base comprises strontium hydroxide. In some embodiments, the strong base comprises barium hydroxide. In some embodiments, a dissolution agent can comprise a chemical such as NaOH to degrade PLA. In some embodiments, the dissolution agent comprises an oxidizing agent, such as to partially or completely degrade the material. In some embodiments, the oxidizing agent is sodium periodate. In some embodiments, an agent can directly break down a scaffolding material into smaller chunks which can then more readily be solvated and be removed from a cell and scaffold composition. In some embodiments, a scaffold removal process can be performed at any stage of a manufacturing process. In some embodiments, a scaffold removal process can be performed after an onset of tissue growth, during a tissue growth period, after tissue growth has completed, but before a beamhouse and tanning process to transform the tissue into leather, during a beamhouse and tanning process, or after a beamhouse and tanning process. Disclosed herein in some embodiments, is a composition comprising a dissolvable scaffold in contact with an extracellular matrix comprising a collagen, wherein a scaffold is in contact with a solvent that can dissolve a scaffold.

In some embodiments, a scaffold further can comprise a polyglycolic acid (PGA), a polybutylene succinate (PBS), a bioabsorbable synthetic polymer, a cellulose, an acetate, an acrylic, a fiber, a linen, a rayon, a velvet, a modacrylic, an olefin polyester, a saran, a vinyon, a wool, a jute, a hemp, a bamboo, a flax, or any combination thereof. In some embodiments, a scaffold further can comprise a bio-based nylon, a bio-based PET, a bio-based PEF, a bio-based polylactic acid (PLA) or any combination thereof. In some embodiments, a scaffold can comprise a nylon 1,6, a nylon 4,6, a nylon 510, a nylon 5,6, a nylon 5,12, a nylon 6, a nylon 6,6, a nylon 11, a nylon 10.10, a nylon 12, or any combination thereof. In some embodiments, a scaffold can comprise a bast fiber, wherein a bast fiber can comprise a flax, a hemp, a linen, a jute, a ramie, a kenaf, a sisal, or any combination thereof.

In some embodiments, any one of the scaffolds provided herein can comprise a thickness of from about 0.1 mm to about 4 mm. In some embodiments, a scaffold can comprise a thickness of from about 1 mm to about 3 mm. In some embodiments, a scaffold can comprise a thickness of about 1 mm. In some embodiments, a scaffold can comprise a thickness of about 2 mm. In some embodiments, a scaffold can comprise a thickness of about 3 mm.

In some embodiments, any one of the scaffolds provided herein can comprise fibers comprising a dtex of about 0.1 dtex to about 50 dtex. In some embodiments, the scaffold comprises fibers comprising a dtex of at least 0.1 dtex (e.g., at least 1 dtex, at least 2 dtex, at least 5 dtex, at least 10 dtex, at least 20 dtex, at least 30 dtex, at least 40 dtex). In some embodiments, the scaffold comprises fibers comprising a dtex of at most 50 dtex (e.g., at most 40 dtex, at most 30 dtex, at most 20 dtex, at most 15 dtex, at most 10 dtex). In some embodiments, the scaffold comprises a dtex of about 0.1 dtex to about 30 dtex. In some embodiments, the scaffold comprises a dtex of about 1 dtex to about 10 dtex. In some embodiments, the scaffold can comprise fibers comprising a dtex of about 6.7 dtex. In some embodiments, the scaffold comprises fibers comprising a dtex of about 1.7 dtex. In some embodiments, the scaffold comprises fibers comprising a dtex of about 3.3 dtex.

In some embodiments, any one of the scaffolds provided herein can comprise fibers comprising a diameter of from about 1 μm to about 100 μm. In some embodiments, the scaffold comprises fibers comprising a diameter of at least 1 μm (e.g., at least 2 μm, at least 5 μm, at least 10 μm, at least 20 μm, at least 30 μm, at least 40 μm, at least 50 μm). In some embodiments, the scaffold comprises fibers comprising a diameter of at most 100 μm (e.g., at most 90 μm, at most 80 μm, at most 70 μm, at most 60 μm, at most 50 μm). In some embodiments, the scaffold comprises fibers comprising a diameter of about 1 μm to about 70 μm. In some embodiments, the scaffold comprises fibers comprising a diameter of about 10 μm to about 50 μm. In some embodiments, the scaffold comprises fibers comprising a diameter of about 10 μm to about 100 μm.

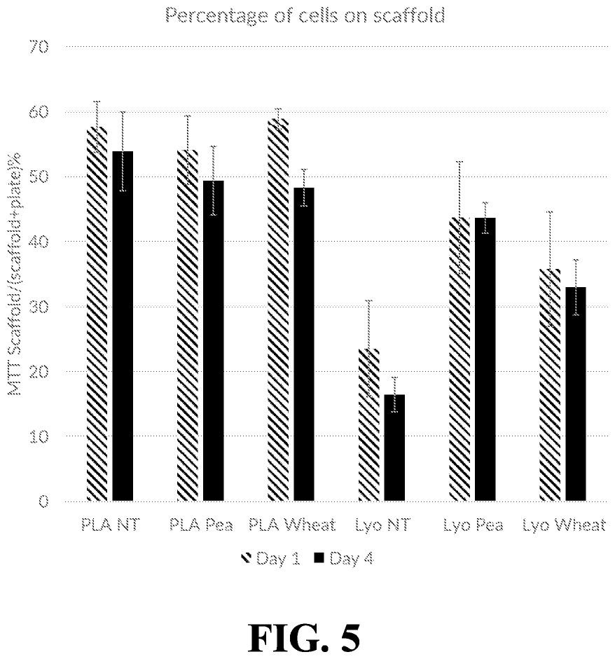

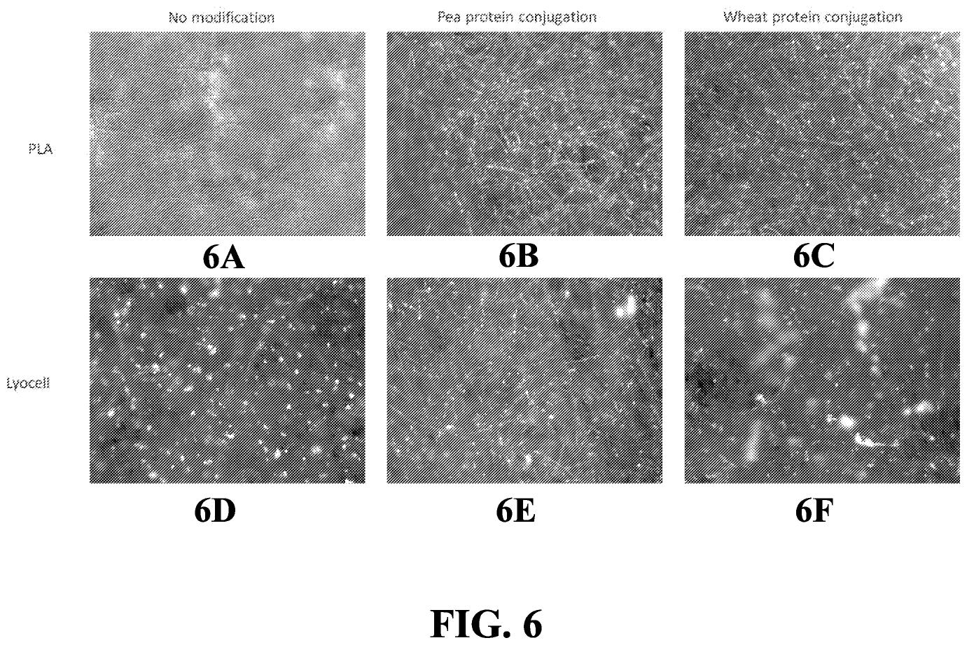

Disclosed herein in some embodiments, is a composition comprising an at least partially coated scaffold in contact with an extracellular matrix comprising a collagen, wherein a scaffold is at least partially coated with a coating comprising a matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, glycidyl trimethylammonium chloride (GTMAC), carbohydrate-binding module, a cellulose binding domain, a starch binding domain, or any combination thereof. In some embodiments, such as described in Example 5, at least partially coating a scaffold with a wheat protein or pea protein increases the percentage of cells on a scaffold in comparison to an un-treated control (FIG. 5). In some embodiments, the conjugation of pea (e.g., sweet pea) protein is more effective than wheat flour protein (FIG. 5, FIG. 6A-F).

In some embodiments, the scaffold is at least partially coated with a carbohydrate-binding module. In some instances, a carbohydrate-binding module is a protein domain found in carbohydrate-active enzymes, such as glycoside hydrolases. In some instances, carbohydrate-binding modules have carbohydrate binding activity. In some embodiments, the carbohydrate-binding module is selected from CBM3, CBM4, CBM6, CBM9, CBM17, CBM20, CBM21, CBM25, CBM28, CBM32, and CBM49. In some instances, a carbohydrate-binding module is a cellulose-binding domain. In some embodiments, the scaffold is at least partially coated with a cellulose-binding domain. In some embodiments, the scaffold comprises cellulose. In some embodiments, the carbohydrate-binding module is a starch-binding module. In some embodiments, the scaffold comprise starch. In some embodiments, the carbohydrate-binding module is associated with an enzyme. In some instances, the enzyme does not hydrolyze the scaffold.

In some embodiments, the scaffold can be modified to comprise a functional group. In some instances, the functional group is native to the scaffold. In other instances, the functional group is a result of a modification. In some embodiments, the scaffold comprises an amine hydroxyl group, a sulfhydryl group, a tyrosyl group, an amine, a sulfate, an aldehyde, a hydrazide, a diazirine, an aryl-azide, an acrylate, an epoxide, or a carboxylic acid group. In some embodiments, the scaffold comprises an amine hydroxyl group. In some embodiments, the scaffold comprises a sulfhydryl group. In some embodiments, the scaffold comprises a tyrosyl group. In some embodiments, the scaffold comprises a carboxylic acid group. In some embodiments, the scaffold comprises an amine. In some embodiments, the scaffold comprises a sulfate. In some embodiments, the scaffold comprises an aldehyde. In some embodiments, the scaffold comprises a hydrazide. In some embodiments, the scaffold comprises a diazirine. In some embodiments, the scaffold comprises an aryl-azide. In some embodiments, the scaffold comprises an acrylate. In some embodiments, the scaffold comprises an epoxide. In some embodiments, the scaffold is at least partially coated with GTMAC. In some embodiments, GTMAC provides primary amines on the surface of the scaffold.

In some embodiments, a coating can comprise a modification. In some embodiments, a modification can comprise a reductive modification, an additive modification, or a combination thereof. In some embodiments, a modification can comprise a reductive modification, an additive modification, or a combination thereof. In some embodiments, a reductive modification can comprise using a base to sever a molecule a bit and create more reactive chemical species to increase attachment or to create some surface roughness. In some embodiments, an additive modification can comprise adding functional ammonia groups with glycidyl trimethylammonium chloride (GTMAC) to increase a positive charge of a surface and increase attachment or grafting a polymer brush to adjust a charge, density, stiffness, or combination thereof of a scaffold surface. In some embodiments, the scaffold is at least partially coated with a coating comprising GTMAC. In some embodiments, the coating comprises primary amines, such as provided by the at least partial coating of GTMAC. In some embodiments, the at least partially coating with a coating comprising GTMAC increases the surface charge of the scaffold.

In some embodiments, the modification comprises hydrolysis. In some embodiments, the modification comprises hydrolysis to expose chemically active groups such as hydroxyls, carboxylic acids, ketones, and the like. In some embodiments, modification exposes hydroxyl groups. In some embodiments, modification exposes carboxylic acid groups. In some embodiments, modification exposes ketone groups. In some embodiments, hydrolysis is completed at high pressure. In some embodiments, hydrolysis is completed in autoclaving conditions. In some embodiments, hydrolysis is completed via enzymatic reactions. In some embodiments, hydrolysis is completed in acidic conditions. In some embodiments, hydrolysis is completed in basic conditions.

In some embodiments, the modification comprises oxidation. In some embodiments, the modification comprises oxidation with sodium periodate or any other appropriate oxidizing agent.

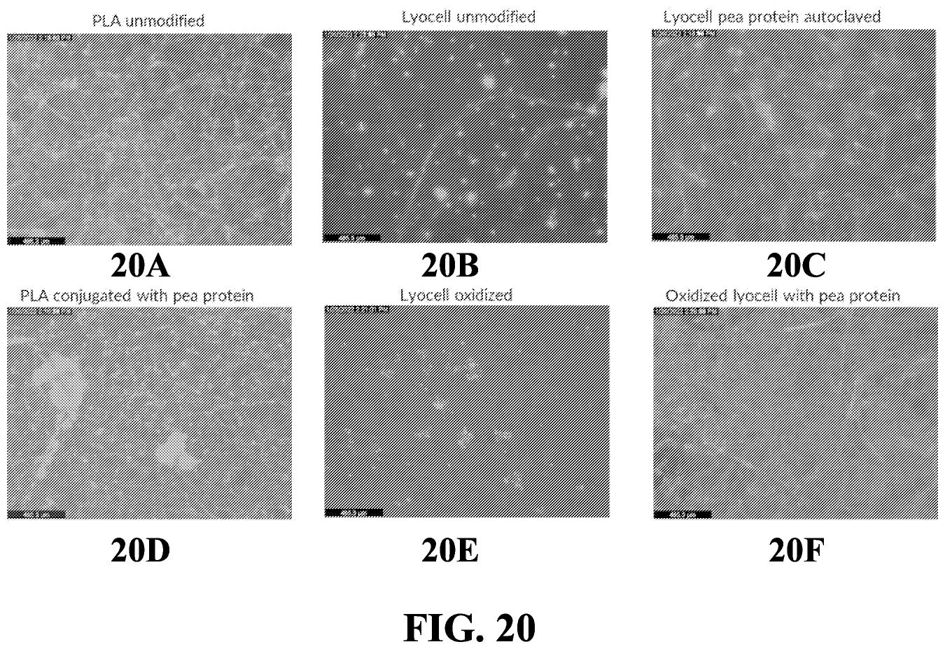

In some embodiments, the modification comprises the Maillard reaction, such as described in Example 5. In some instances, a scaffold is soaked in a protein rich solution under autoclaving conditions to treat the surface. In some instances, the Maillard reaction aids in assisting in pea protein conjugation to Lyocell (FIG. 20C) with the improvement in cell spreading compared to an unmodified scaffold (FIG. 20B). In some instances, the scaffolds herein, such as described in Example 5, are oxidized with sodium periodate. In some instances, a combination of modification methods are used, such as a combination of the Maillard reaction and oxidation, such may result in cell spreading (FIG. 20F).

In some embodiments, in any of the compositions provided herein, the collagen is associated with the scaffold. In some embodiments, associated comprises bonding. In some embodiments, bonding comprises covalent or non-covalent bonding. In some embodiments, in any of the compositions provided herein, the collagen is bonded to the scaffold. In some embodiments, association of the collagen with the scaffold comprises crosslinking of the collagen with the scaffold.

In some embodiments, the collagen is associated, such as bonded, with the scaffold through non-specific adsorption. In some instances, the non-specific adsorption comprises Van der Waals interactions, hydrogen bonding, depletion interactions, electrostatic interactions, or a combination thereof. In some embodiments, the collagen is associated with the scaffold through Van der Waals interactions. In some embodiments, the collagen is associated with the scaffold through hydrogen bonding. In some embodiments, the collagen is associated with the scaffold through depletion interactions. In some embodiments, the collagen is associated with the scaffold through electrostatic interactions. In some instances, at least partially coating the surfaces with a coating as described elsewhere herein can increase the strength of non-specific adsorption. In other embodiments, modification of the scaffold, such as a modification as described elsewhere herein, increases a strength of a non-specific adsorption of the collagen to the scaffold.

In some embodiments, the collagen is associated with the scaffold through covalent interactions. In some embodiments, the collagen is associated with the scaffold through covalent bonding. In some instances, modification, such as a modification as described elsewhere herein, allows for covalent interaction, such as covalent bonding, of the collagen to the scaffold.

In some embodiments, the scaffold comprises a carbodiimide. In some embodiments, the scaffold comprises an N-hydroxysuccinimide ester (NHS ester). In some embodiments, the collagen comprises a carbodiimide. In some embodiments, the collagen comprises an N-hydroxysuccinimide ester (NHS ester). In some embodiments, the carbodiimide is N,N′-Dicyclohexylcarbodiimide (DCC). In some embodiments, the carbodiimide is 1-ethyl-3-(3-(dimethylaminopropyl)carbodiimide (EDC). In some embodiments, the collagen is associated with, such as forms a covalent bond with, the scaffold through EDC/NHS coupling, such as between an NHS ester and a carbodiimide. In some embodiments, the fibroblast and/or fibroblast-like cell comprise a carbodiimide. In some embodiments, the fibroblast and/or fibroblast-like cell comprise an N-hydroxysuccinimide ester (NHS ester). In some embodiments, the fibroblast and/or fibroblast-like cell is associated with, such as forms a covalent bond with, the scaffold through EDC/NHS coupling.

In some embodiments, the scaffold comprises a click chemistry moiety. In some embodiments, the scaffold comprises an azide. In some embodiments, the scaffold comprises an alkyne. In some embodiments, the collagen comprises a click chemistry moiety. In some embodiments, the collagen comprises an azide. In some embodiments, the scaffold comprises an alkyne. In some embodiments, the collagen is associated with, such as forms a covalent bond with, the scaffold through a click chemistry reaction, such as between an azide and an alkyne. In some embodiments, the fibroblast and/or fibroblast-like cell comprise a click chemistry moiety. In some embodiments, the fibroblast and/or fibroblast-like cell comprise an azide. In some embodiments, the fibroblast and/or fibroblast-like cell comprise an alkyne. In some embodiments, the fibroblast and/or fibroblast-like cell is associated with, such as forms a covalent bond with, with the scaffold through a click chemistry reaction, such as between an azide and an alkyne.

In some embodiments, the scaffold comprises a Michael donor. In some embodiments, the scaffold comprises a Michael acceptor. In some embodiments, the collagen comprises a Michael donor. In some embodiments, the scaffold comprises a Michael acceptor. In some embodiments, the collagen is associated with the scaffold through coupling of the Michael donor and the Michael acceptor. In some embodiments, the fibroblast and/or fibroblast-like cell comprise a Michael donor. In some embodiments, the fibroblast and/or fibroblast-like cell comprise a Michael acceptor. In some embodiments, the fibroblast and/or fibroblast-like cell is associated with the scaffold through coupling of the Michael donor and the Michael acceptor. In some embodiments, the Michael donor comprises an enolate. In some embodiments, the Michael acceptor comprises an α,β-unsaturated carbonyl.

In some embodiments, the scaffold comprises a thiol. In some embodiments, the scaffold comprises a maleimide. In some embodiments, the collagen comprises a thiol. In some embodiments, the collagen comprises a maleimide. In some embodiments, the collagen is associated with, such as forms a covalent bond with, the scaffold through coupling of the thiol and the maleimide. In certain instances, the collagen is associated with the scaffold in the presence of (sulfosuccinimidyl 4-(N-maleimidomethyl)cyclohexane-1-carboxylate) (sulfo-SMCC), wherein both the scaffold and collagen comprise a thiol. In some embodiments, the fibroblast and/or fibroblast-like cell comprises a thiol. In some embodiments, the fibroblast and/or fibroblast-like cell comprises a maleimide. In some embodiments, the fibroblast and/or fibroblast-like cell is associated with, such as forms a covalent bond with, the scaffold through coupling of the thiol and the maleimide. In certain instances, the fibroblast and/or fibroblast-like cell is associated with the scaffold in the presence of (sulfosuccinimidyl 4-(N-maleimidomethyl)cyclohexane-1-carboxylate) (sulfo-SMCC), wherein both the scaffold and fibroblast and/or fibroblast-like cell comprise a thiol.

In some embodiments, the collagen is associated with, such as forms a covalent bond with, the scaffold through a Maillard reaction, such as a reaction between reducing sugars and proteins. In some embodiments, the fibroblast and/or fibroblast-like cell is associated with, such as forms a covalent bond with, the scaffold through a Maillard reaction, such as a reaction between reducing sugars and proteins.

In some embodiments, a scaffold can comprise a thermoplastic polymer which can be subsequently substantially removed from an extracellular matrix prior to a tanning. In some embodiments, a thermoplastic polymer can comprise a polyvinyl alcohol (PVA), and wherein a method can comprise substantially removing a PVA prior to a tanning by contacting a PVA with water having a temperature of about 18° C. to about 90° C. In some embodiments, a thermoplastic polymer can comprise a Polylactic acid (PLA) and wherein a method can comprise substantially removing a PLA prior to a tanning by contacting a PLA with a solvent to remove a PLA. In some embodiments, a solvent can comprise a benzyl group, an ethyl group, a haloalkane, a benzylamine, or any combination thereof. In some embodiments, a scaffold can comprise a dissolvable scaffold which can be substantially removed by contacting a scaffold with a solvent. In some embodiments, a method of making a scaffold can comprise needle punching a scaffold using a needle loom to entangle fibers in a non-woven scaffold material. In some embodiments, a needle loom can comprise a barbed needle. In some embodiments, a needle punching can create pores in a scaffold material. In some embodiments, a method of making a scaffold can comprise at least partially coating a scaffold with a coating comprising a matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, or any combination thereof.

Disclosed herein in some embodiments, is a composition comprising a scaffold in contact with an extracellular matrix. In some embodiments, in an extracellular matrix can comprise a collagen. In some embodiments, a scaffold and cell composition can have at least one component of native skin such as melanocytes, hair follicles, sweat glands and nerve endings. In certain cases, a cruelty-free leather can be distinguished from normal native skin by its lack of at least one of these components. In some embodiments, displaying abnormal phenotypes or having at least one cell with an altered genotype, a scaffold and cell composition leather can include all of these components.

Disclosed herein in some embodiments, is a composition comprising an isolated animal cell in contact with a scaffold. In some embodiments, an isolated animal cell can be an isolated animal fibroblast or fibroblast-like cell. In some embodiments, an extracellular matrix can be produced by an isolated animal fibroblast or fibroblast-like cell. In some embodiments, an isolated animal cell can be an immortalized isolated animal cell. In some embodiments, an immortalized isolated animal cell can be grown past a Hayflick limit. In some embodiments, an immortalized isolated animal cell can be grown past about 40 cell divisions, about 50 cell divisions, or about 60 cell divisions. In some embodiments, an isolated animal cell can be a bovine or porcine cell. In some embodiments, an isolated animal cell can comprise a human cell. In some embodiments, a composition can be at least partially decellularized. In some embodiments, at least partially decellularized can comprise substantially no intact cells being present in a composition.

In some embodiments, additional components can be added to a scaffold and cell composition. Such additional components can include myoepithelial cells, duct cells, secretory cells, alveolar cells, Langerhans cells, Merkel cells, adhesions, mammary glands, or any mixture thereof. In some embodiments, a cruelty-free leather can comprise one or more of: neural cells, connective tissue (including bone, cartilage, cells differentiating into bone forming cells and chondrocytes, and lymph tissues), epithelial cells (including endothelial cells that form linings in cavities and vessels or channels, exocrine secretory epithelial cells, epithelial absorptive cells, keratinizing epithelial cells, and extracellular matrix secretion cells), and undifferentiated cells (such as embryonic cells, stem cells, and other precursor cells).

In some embodiments, a scaffold and cell composition can comprise hair follicles. A hair follicle can comprise one or more structures, including papilla, matrix, root sheath, bulge, infundibulum, an arrector pili muscle, a sebaceous gland, an apocrine sweat gland, or any combination thereof. A hair follicle can comprise one or more hair follicle cells, including dermal papilla cell, outer root sheath cell, or any combination thereof. In some embodiments, a hair follicle can be in an epidermal layer. In some embodiments, a hair follicle can be in a dermal layer. In some embodiments, a hair follicle cell can be differentiated from a progenitor, e.g., a stem cell. In some embodiments, at least about 1%, 2%, 3%, 4%, 5%, 6%, 7%, 8%, 9%, 10%, 20%, 30%, 40%, 50%, 60%, 70%, 80%, 90%, 100% of hair follicle cells can be differentiated from an induced pluripotent stem cell.

In some embodiments, a scaffold and cell composition can be devoid of hair, blood vessels, sebaceous glands, hair follicle, oil glands, nerve, or any combination thereof immediately prior to or after a tanning.

### Cell Types

In some embodiments, at least a portion of one or more cells in a cruelty-free leather can be differentiated from a progenitor cell, such as a stem cell. In some embodiments, a cruelty-free leather can be generated from an engineered cell, or a tissue comprising an engineered cell as disclosed herein. In some embodiments, a fibroblast in a cruelty-free leather can be differentiated from a stem cell. In some embodiments, a keratinocyte in a cruelty-free leather can be differentiated from a stem cell. In some embodiments, a melanocyte in a cruelty-free leather can be differentiated from a stem cell.

In some embodiments, a stem cells can comprise an embryonic stem cell (ESC), an adult stem cell, a somatic stem cell, a tissue-specific stem cell, a mesenchymal stem cell, an induced pluripotent stem cell (iPSC), or any combination thereof. In some embodiments, a stem cell can be totipotent, pluripotent or multipotent. In some embodiments, a stem cell can comprise an adult stem cell, a cord blood stem cell, or a combination thereof. Embryonic stem cells can be derived from fertilized embryos that may be less than one week old. Induced pluripotent stem cells can be obtained through an induced expression of one or more of Oct3, Oct4, Sox2, Klf4, TERT, Bmi1, CcnD1, Cdk4, SV40 large T antigen, c-Myc, a fragment of any of these genes, in any somatic cell. In some embodiments a somatic cell can comprise an adult somatic cell. In some embodiments, a somatic cell can comprise a fibroblast. In some embodiments, an exogenous vector can comprise or encode a gene to induce pluripotency. In some embodiments, an exogenous vector can comprise a plasmid. In some embodiments, an induced pluripotent stem cell can be obtained by an active protein product or a biologically active fragment thereof. In some embodiments, one or more other genes can also be induced for reprograming a somatic cell to an induced pluripotent stem cell. In some embodiments a gene for inducing pluripotency can comprise NANOG, UTF1, LIN28, SALL4, NR5A2, TBX3, ESSRB, DPPA4, SV40LT, REM2, MDM2, and cyclin D1. In some embodiments, a gene can be from a human. In some embodiments, a gene can be from a mammal, a bird, a reptile, an amphibian, a fish, an invertebrate, or any combination thereof.

In some embodiments, various delivery methods can be used to modulate an expression of a gene to reprogram a somatic cell to an iPSC. In some embodiments, an exemplary delivery method can include a naked DNA delivery, an adenovirus vector, an electrical delivery, a chemical delivery, a mechanical delivery, a polymer-based system, a microinjection, a retrovirus vector (e.g., MMLV-derived retroviruses), a lentivirus vector (e.g., excisable lentiviruses), or any combination thereof. In some embodiments, a somatic cell can comprise an adult somatic cell. In some embodiments, a somatic cell can be transfected with a vector for delivery of a gene inducing pluripotency. In some embodiments, a vector can comprise a viral vector. In some embodiments, a vector can comprise a retroviral vector. In some embodiments, a gene inducing pluripotency can comprise Oct3, Oct4, Sox2, Klf4, TERT, Bmi1, CcnD1, Cdk4, SV40 large T antigen, c-Myc, a fragment of any of these, or any combination thereof. In some embodiments, a Sendai virus can be used as a delivery system. In some embodiments, a somatic cell can comprise an adult somatic cell. In some embodiments, a somatic cell can be transfected with an extrachromosomal vector. In some embodiments, an extrachromosomal vector can comprise a plasmid. In some embodiments, an extrachromosomal vector can deliver Oct3, Oct4, Sox2, Klf4, TERT, Bmi1, CcnD1, Cdk4, SV40 large T antigen, c-Myc, a fragment of any of these, or any combination thereof.

Disclosed herein in some embodiments are compositions comprising an isolated cell that can be directed to producing a cruelty-free leather, a cruelty-free leather, an isolated epidermal layer, an isolated dermal layer, a layered structure, a product produced therefrom, methods of producing the same, or any combination thereof. Disclosed herein in some embodiments, are methods and compositions comprising an isolated cell. In some embodiments, an isolated cell can comprise a cell derived from an animal. In some embodiments, a cell can be derived from a mammal, a bird, a reptile, an amphibian, a fish, an invertebrate, or any combination thereof. In some embodiments, a cell can be derived from an antelope, a bear, a beaver, a bison, a boar, a camel, a caribou, a cat, a cattle, a deer, a dog, an elephant, an elk, a fox, a giraffe, a goat, a hare, a human, a horse, an ibex, a kangaroo, a lion, a llama, a lynx, a mink, a moose, an oxen, a peccary, a pig, a rabbit, a rhino, a seal, a sheep, a lamb, a squirrel, a tiger, a whale, a wolf, a yak, or a zebra. In some embodiments, an animal can be a primate, a bovine, an ovine, a porcine, an equine, a canine, a feline, a rodent, a lagomorph, a fish, a bird or a reptile. In some embodiments, a cell can be derived from a bird. In some embodiments, a bird can comprise a chicken, a duck, an emu, a goose, a grouse, an ostrich, a pheasant, a pigeon, a quail, or a turkey. In some embodiments, a cell can be derived from a reptile such as a turtle, a snake, a lizard, an amphibian, a crocodile, or an alligator. In some embodiments, a cell can be derived from an amphibian. In some embodiments, an amphibian can comprise a frog, a toad, a salamander, or a newt. In some embodiments, a cell can be derived from a fish. In some embodiments, a fish can comprise an anchovy, a bass, a catfish, a carp, a cod, an eel, a flounder, a fugu, a grouper, a haddock, a halibut, a herring, a mackerel, a mahi-mahi, a manta ray, a marlin, an orange roughy, a perch, a pike, a pollock, a salmon, a sardine, a shark, a snapper, a sole, a stingray, a swordfish, a tilapia, a trout, a tuna, or a walleye. In some embodiments, an isolated cell can be obtained from a biopsy. In some embodiments, an isolated cell can comprise an isolated animal cell. In some embodiments, an isolated animal cell can comprise a primate cell, a bovine cell, an ovine cell, a porcine cell, an equine cell, a canine cell, a feline cell, a rodent cell, a bird cell, a marsupial, a reptile, or a lagomorph animal cell. In some embodiments, an isolated animal cell can be a genetically engineered cell. A cell can comprise a cell line with a plurality of cells. In some embodiments, an isolated animal cell can be an immortalized cell. In some embodiments, a cell can comprise a human cell, a fibroblast, a stem cell, or any combination thereof. In some embodiments, a cell can be a fat tissue derived cell (e.g., an adipocyte), a chondrocyte, an osteocyte, an osteoblast, a myofibroblast, a satellite cell, a myoblast, a myocyte, a keratinocyte, a corneocyte, a melanocyte, a Langerhans cell, a basal cell, a smooth muscle cell, an umbilical cord cell, a pluripotent stem cell, a mesenchymal stem cell, an embryonic stem cell, or any combination thereof. In some embodiments, a cell can produce an extracellular matrix (ECM). In some embodiments, an ECM protein can comprise a collagen, a collagen type I, a collagen type III, an elastin, a fibronectin, a laminin or any combination thereof. In some embodiments, an engineered tissue can comprise a cell described herein. In some embodiments, an isolated animal cell can comprise an engineered isolated animal cell. In some embodiments, an engineered isolated animal cell can comprise a genetically engineered cell. In some embodiments, a genetically engineered cell can comprise an exogenous polynucleotide. In some embodiments, a genetically engineered cell can comprise a gene for collagen production. In some embodiments, a collagen gene can be P4HA, P4HB, COL1A1, COL1A2, COL2A1, COL3A1 or any combination thereof. In some embodiments, a collagen gene can have an altered promotor that can change an expression of a collagen gene, e.g., increase or decrease. In some embodiments, a collagen gene can be from a human. In some embodiments, a collagen gene can be from an animal, a mammal, a bird, a reptile, an amphibian, a fish, an invertebrate, or any combination thereof. In some embodiments, an exogenous gene or a plurality of exogenous genes can cause immortalization. In some embodiments, an exogenous gene can comprise hTERT, TERT, Bmi1, CcnD1, a mutant of Cdk4, Cdk4, TAg (SV40 large T), SV40, c-myc, H-ras, Ela, c-mMycER™, E6, E7, HER-2, SRC, EGFR, Abl, Atk02, Aml1, Axl, Bcl, Dbl, EGFR, ERBB, Ets-1, Fms, Fos, Fps, Gli, Gsp, Her2, Hox11, Hst, Il-3, Int-2, Jun, Kit, KS3, K-SAM, Lbc, Lck, L-myc, Lyl-1, Lyt-10, Mas, MDM-2, Mll, Mos, Myb, Neu, N-Myc, Ost, Pax-5, Pim-1, PRAD-1, Ras-K, Ras-N, Ret, Ros, Ski, Sis, Set, Src, Tal1, Tan1, Tiam1, Tsc2, Trk, or any combination thereof. In some embodiments, an isolated animal cell can comprise an immortalized cell, a tissue developed therefrom, or any combination thereof. In some embodiments, an exogenous polynucleotide can encode: (i) a polypeptide which interacts with a tumor suppressor protein or fragment thereof and can alter an activity of a tumor suppressor protein or fragment thereof, (ii) a polynucleotide that can encode a polypeptide which interacts with a tumor suppressor protein or fragment thereof, or (iii) a combination of (i) and (ii). In some embodiments, an activity of a tumor suppressor protein or fragment thereof can be measured by an in vitro assay. In some embodiments, an immortalized cell can have a random mutation or a plurality of mutations. In some embodiments, a mutation can be generated by UV mutagenesis, chemical mutagenesis, or any combination thereof. In certain cases, an immortalized cell can have a targeted mutation, for example, a targeted mutation may be made by a CRISPR system. In some embodiments, a mutation can be in a cell cycle gene, an oncogene, a metabolic gene, or any combination thereof. In some embodiments, an immortalized cell can have a mutation in a gene, a promotor region, an intragenic region, an intergenic region, or any combination thereof. In some embodiments, a gene can comprise an oncogene, a cell cycle gene, or a combination thereof. In some embodiments, an immortalized cell can have increased or decreased expression of an oncogene or genes involved in a regulation of cell proliferation. In some embodiments, an immortalized cell can be grown past about 30 cell divisions, about 40 cell divisions, about 50 cell divisions, about 60 cell divisions, about 70 cell divisions, about 80 cell divisions, about 90 cell divisions, about 100 cell divisions, about 150 cell divisions, about 200 cell divisions, about 250 cell divisions, about 300 cell divisions, about 350 cell divisions, about 400 cell divisions, about 450 cell divisions, about 500 cell divisions, about 550 cell divisions, about 600 cell divisions, about 650 cell divisions, about 700 cell divisions, about 750 cell divisions, about 800 cell divisions, about 850 cell divisions, about 900 cell divisions, about 950 cell divisions, about 1000 cell divisions, about 5,000 cell divisions, about 10,000 cell divisions, about 50,000 divisions, or about 100,000 cell divisions. In some embodiments, a Hayflick limit or Hayflick number can comprise a finite number of cell doublings which a primary cell can be grown to. In some embodiments, an immortalized cell can be grown past a Hayflick limit. In some embodiments, an immortalized cell line can be grown enough to generate at least 1 million square feet per year of cruelty-free leather.

In some embodiments, in any of the compositions provided herein, the scaffold may be seeded with any appropriate collagen producing cell. In some embodiment, the scaffold may be seeded with a primary cell. In some embodiments, the scaffolds provided herein may be seeded with a collagen producing cell comprising a fibroblast, a keratinocyte, a melanocyte, an epithelial cell, a comeocyte, a Langerhans cell, a basal cell, or a combination thereof. In some embodiments, the scaffolds provided herein may be seeded with a fibroblast. In some embodiments, the scaffold provided herein may be seeded with a keratinocyte. In some embodiments, the scaffold provided herein may be seeded with a melanocyte. In some embodiments, the scaffold provided herein may be seeded with an epithelial cell. In some embodiments, the scaffold provided herein may be seeded with a comeocyte. In some embodiments, the scaffold provided herein may be seeded with a Langerhans cell. In some embodiments, the scaffold provided herein may be seeded with a basal cell.

### Methods of Making Cruelty-Free Leather

Disclosed herein in some embodiments, is a method of making a composition disclosed herein, comprising seeding an isolated animal fibroblast or fibroblast-like cell onto a scaffold to form a composition. In some embodiments, a cell layer can be formed by preparing a plurality of multicellular bodies comprising one or more type of isolated cells and arranging such multicellular bodies to form a cell layer. In some embodiments, a cell layer can be formed by adjacently arranging a plurality of multicellular bodies, wherein a plurality of multicellular bodies can be fused to form a planar layer. In some embodiments, a cell can be grown three dimensionally. In some embodiments, a cell can be grown in suspension. In some embodiments, forming a cell layer can comprise using a scaffold. In some embodiments, a cell layer can be formed by arranging a plurality of isolated cells, multicellular bodies, or a combination thereof on a scaffold. In some embodiments, a forming step can comprise arranging or placing multicellular bodies or seeding a plurality of isolated animal cells on a support substrate that allows a multicellular body, plurality of isolated animal cells, or a combination thereof to fuse to form a layer (e.g., a substantially planar layer). In some embodiments, a multicellular body or a layer can be arranged horizontally and/or vertically adjacent to one another. In some embodiments, forming a cell layer can be performed without a scaffold. In some embodiments, a cell layer can be formed on a scaffold, and subsequently a scaffold can be at least partially removed. In some embodiments, a scaffold can comprise a support substrate. In some embodiments, a support substrate can be permeable to fluids, gasses, and nutrients and allows cell culture medium to contact all surfaces of a multicellular bodies and/or layers during arrangement and subsequent fusion. In some embodiments, a support substrate can be made from natural biomaterials such as collagen, fibronectin, laminin, and other extracellular matrices. In some embodiments, a support substrate can be made from synthetic biomaterials such as hydroxyapatite, alginate, agarose, polyglycolic acid, polylactic acid, and their copolymers. In some embodiments, a support substrate can be solid, semisolid, or a combination of solid and semisolid support elements. In some embodiments, a support substrate can be planar to facilitate production of planar layers. In some embodiments, a support substrate can be raised or elevated above a non-permeable surface, such as a portion of a cell culture environment (e.g., a Petri dish, a cell culture flask, etc.) or a bioreactor. In some embodiments, a permeable, elevated support substrate can contribute to prevention of premature cell death, can contribute to enhancement of cell growth, and can facilitate fusion of multicellular bodies to form layers. In some embodiments, a cell layer can comprise a dermal layer. In some embodiments, a cruelty-free leather can comprise a dermal layer, or an at least partially decellularized portion thereof. In some embodiments, a dermal layer can be an engineered dermis equivalent, e.g., an artificial dermal layer formed in vitro. In some embodiments, a dermal layer can comprise cells of a connective tissue. In some embodiments, a dermal layer can comprise a fibroblast or fibroblast-like cell. In some embodiments, a fibroblast or fibroblast-like cell in a dermal layer can express one or more markers including, but not limited to, a cluster of differentiation 10 (CD10), a cluster of differentiation 73 (CD73), a cluster of differentiation 44 (CD44), a cluster of differentiation 90 (CD90), a cluster of differentiation 105 (CD105), a type I collagen, a type III collagen, a prolyl-4-hydroxylase beta fibroblast, or a combination thereof. In some embodiments, a dermal layer can comprise other types of cells, such as immune cells, macrophages, adipocytes, or a combination thereof. In some embodiments, a dermal layer can comprise an engineered cell. In some embodiments, a cell layer can comprise an immortalized cell, a bovine cell, a fibroblast cell, or any combination thereof. In some embodiments, a cell layer can comprise an immortalized bovine fibroblast. In some embodiments, a dermal layer can comprise a matrix component in addition to a cell. In some embodiments, a matrix component can include any one or more of collagen, elastin, an extrafibrillar matrix, an extracellular gel-like substance primarily composed of glycosaminoglycans, proteoglycans, glycoproteins, or any combination thereof. In some embodiments, an extracellular gel-like substance primarily composed of glycosaminoglycans can comprise a hyaluronan. In some embodiments, a dermal layer can comprise a matrix support. In some embodiments, a matrix support can be a scaffold. In some embodiments, a matrix support can comprise a contracted collagen gel. In some embodiments, a pure collagen matrix can be a polyglycolic acid mesh or collagen and glycosaminoglycan matrix covered with a silastic membrane (C-GAG), a biopolymer, or any combination thereof. In some embodiments, a biopolymer can comprise chitosan. In some embodiments, a matrix can be seeded with fibroblasts. In some embodiments, seeding with a fibroblast can give rise to an organotypic model. In some embodiments, a cell layer can comprise a naturally derived dermis, a keratinocyte, or a combination thereof. In some embodiments, a naturally derived dermis can be obtained from an allogenic cadaver skin. In some embodiments, a keratinocyte can form a sheet of keratinocytes. In some embodiments, a cell layer can comprise a lyophilized devitalized dermis from cadaver skin to support a keratinocyte sheet.

In some embodiments, the methods provided herein may further comprise cell growth under rocking conditions. In some embodiments, such as described in Example 1, rocking conditions result in the consumption of more glucose. In some embodiments, the methods provided herein may further comprise cell growth under static conditions.

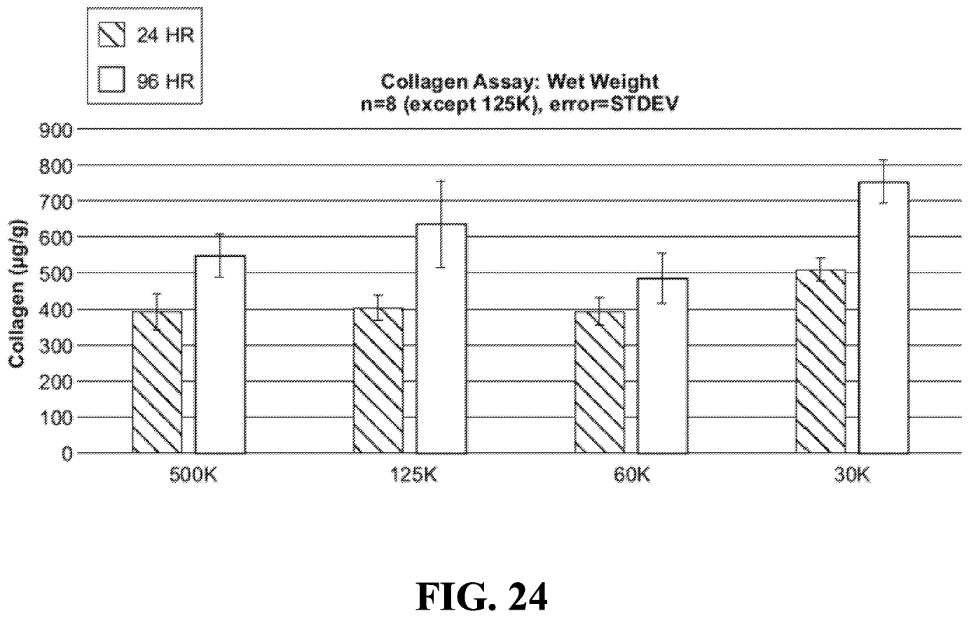

In some embodiments, the methods provided herein further comprise seeding cells at any appropriate seeding density as determined by one skilled in the art. In some embodiments, cells are seeded at a density of at least 30 k cells/cm² (e.g., at least 50 k cells/cm², at least 62.5 k cells/cm², at least 100 k cells/cm², at least 125 k cells/cm², at least 200 k cells/cm², at least 400 k cells/cm², at least 600 k cells/cm², at least 800 k cells/cm², at least 1M cells/cm²). In some embodiments, cells are seeded at a density of at most 2M cells/cm² (e.g., at most 1.5M cells/cm², at most 1.25M cells/cm², 1M cells/cm², at most 800 k cells/cm², at most 600 k cells/cm², at most 400 k cells/cm², at most 200 k cells/cm², at most 100 k cells/cm²). In some embodiments, cells are seeded at a density of no more than 200 k cells/cm². In some embodiments, cells are seeded at a density of about 30 k cells/cm² to about 2M cells/cm². In some embodiments, cells are seeded at a density of about 62.5 k cells/cm² to about 1M cells/cm². In some embodiments, cells are seeded at a density of about 62.5 k cells/cm² to about 200 k cells/cm². In some instances, lower seeding densities (e.g., 30 k cells/cm²) lead to higher collagen production than higher seeding densities (FIG. 24).

In some embodiments, the methods provided herein further comprise incubating for any suitable period of time. In some embodiments, the methods comprise incubating for at least 1 week (e.g., at least 1.5 weeks, at least 2 weeks, at least 3 weeks, at least 4 weeks, at least 6 weeks, at least 8 weeks). In some embodiments, the methods comprise incubating for at most 12 weeks (e.g., at most 10 weeks, at most 8 weeks, at most 6 weeks, at most 4 weeks, at most 2 weeks, at most 1 week). In some embodiments, the methods comprise incubating for about 1 week to about 12 weeks. In some embodiments, the methods comprise incubating for about 4 weeks to about 8 weeks. In some embodiments, the methods comprise incubating for about 4 weeks. In some embodiments, the methods comprise incubating for about 6.5 weeks. In some embodiments, the methods comprise incubating for about 8 weeks.

In some embodiments, the methods provided herein further comprise soaking the scaffold in a protein containing solution (e.g., serum) prior to culturing. In some embodiments, soaking comprises soaking the scaffold in fetal bovine serum. In some embodiments, soaking may be important for cell attachment and proliferation. In some instances, soaking allows for the adsorption of sticky proteins to the surface allowing for increased cell attachment.

In some embodiments, a thickness of a dermal layer can be engineered to fit a function or use of a cruelty-free leather. In some embodiments, a dermal layer can have a thickness from about 0.01 mm to about 50 mm. In some embodiments, a dermal layer can have a thickness from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a dermal layer can have a thickness from about 0.02 mm to 5 mm. For example, a dermal layer can have a thickness from about 0.1 mm to 0.5 mm. In some embodiments, a dermal layer can have a thickness from about 0.2 mm to 0.5 mm. In some embodiments, a thickness of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a thickness of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a thickness of at least about 50 mm.

In some embodiments, a length of a dermal layer can be engineered to fit a function or use of a cruelty-free leather. In some embodiments, a dermal layer can have a length from about 0.01 mm to about 50 m. In some embodiments, a dermal layer can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a dermal layer can have a length from about 0.02 mm to 5 mm. For example, a dermal layer can have a length from about 0.1 mm to 0.5 mm. In some embodiments, a dermal layer can have a length from about 0.2 mm to 0.5 mm. In some embodiments, a length of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a length of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a length of at least about 50, 60, 70, 80, 90, 100, 200, 300, 400, 500, 700, 1000 mm. In some embodiments, a dermal layer can have a length of at least about 50, 60, 70, 80, 90, 100, 200, 300, 400, 500, 600, 700 cm. In some embodiments, a dermal layer can have a length of at least about 50, 60, 70, 80, 90, 100, 200, 300, 400 m.

In some embodiments, a width of a dermal layer can be engineered to fit a function or use of a cruelty-free leather. In some embodiments, a dermal layer can have a width from about 0.01 mm to about 50 m. In some embodiments, a dermal layer can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, a dermal layer can have a width from about 0.02 mm to 5 mm. In some embodiments, a dermal layer can have a width from about 0.1 mm to 0.5 mm. In some embodiments, a dermal layer can have a width from about 0.2 mm to 0.5 mm. In some embodiments, a width of a dermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a width of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, a dermal layer can have a width of at least about 50, 60, 70, 80, 90, 100, 200, 300, 400, 500, 700, 1000 mm. In some embodiments, a dermal layer can have a width of at least about 50, 60, 70, 80, 90, 100, 200, 300, 400, 500, 600, 700 cm. In some embodiments, a dermal layer can have a width of at least about 50, 60, 70, 80, 90, 100, 200, 300, 400 m.

In some embodiments, a cruelty-free leather can comprise one or more dermal layers. In some embodiments, a cruelty-free leather can have at least 1, 2, 3, 4, 5, 6, 7, 8, 9, 10, 12, 14, 16, 18, 20, 40, 60, 80, or 100 dermal layers. In some embodiments, when a cruelty-free leather can comprise more than one dermal layer, a dermal layer can be placed upon another dermal layer. In some embodiments, a cruelty-free leather can comprise two dermal layers, e.g., a first dermal layer and a second dermal layer. In some embodiments, a first dermal layer can be placed upon a second dermal layer.

In some embodiments, a dermal layer or an at least partially decellularized portion thereof can be stratified, e.g., having a plurality of sublayers. In some embodiments, a sublayer can have different compositions, e.g., different concentrations of a fiber. In some embodiments, a sublayer of a dermal layer or an at least partially decellularized portion thereof can have a different thickness, density, or a combination thereof. In some embodiments, a dermal layer or an at least partially decellularized portion thereof can have a papillary dermal layer, a reticular dermal layer, an at least partially decellularized portion of any of these, or any combination thereof. In some embodiments, a papillary dermal layer or an at least partially decellularized portion thereof can comprise loose areolar connective tissue, loosely arranged fibers, an at least partially decellularized portion of these, or any combination thereof. In some embodiments, a loosely arranged fiber can comprise a collagen fiber. In some embodiments, a reticular dermal layer can comprise a dense irregular connective tissue, including collagen fibers and dermal elastic fibers.

In some embodiments, a dermal layer or an at least partially decellularized portion thereof can comprise a free collagen matrix or lattice, which can be contractile in all directions, and homogeneous. In some embodiments, fibroblasts (e.g., immortalized bovine fibroblasts), and where appropriate other types of cells of a dermis, can be distributed in a continuous collagen gel. In some embodiments, a dermis equivalent can comprise at least one matrix of collagen type I in which fibroblasts can be distributed. In some embodiments, a dermis equivalent can also contain other extracellular matrix constituents. In some embodiments, an extracellular matrix constituent can include collagens, e.g., collagen IV, laminins, entactin, fibronectin, proteoglycans, glycosaminoglycans or hyaluronic acid. In some embodiments, a dermal layer can contain collagen type IV and laminin, entactin, or a combination thereof. In some embodiments, a concentration of these various constituents can be adjusted. For example, in some embodiments, a concentration of laminin can be from about 1% to about 15% of a final volume. In some embodiments, a concentration of collagen IV can be from about 0.3% to about 4.5% of a final volume. In some embodiments, a concentration of entactin can be from about 0.05% to about 1% of a final volume. In some embodiments, a collagen can be a collagen of bovine origin, of rat origin, of fish origin, any other source of natural collagen or collagen produced by genetic engineering which allows contraction in a presence of fibroblasts, or any combination thereof. In some embodiments, a collagen can be from an unnatural source. In some embodiments, a matrix can be a gel of collagen which may not be taut, obtained by contraction both horizontally and vertically, which does not impose a preferential organization of fibroblasts. In some embodiments, a matrix, also termed “free”, may not adhere to a support and volumes thereof can be modified without limit, conferring on it a varying thickness and diameter. In some embodiments, a thickness of a dermis equivalent can be at least 0.05 cm and, In some embodiments, approximately from 0.05 to 2 cm. In some embodiments, a thickness can also be increased without harming an advantageous property of a skin equivalent or cruelty-free leather. In some embodiments, a thickness can be from about 3 mm to about 20 cm or more. In some embodiments, a cruelty-free leather can comprise only dermal layers.

In some embodiments, a cell and scaffold composition can comprise an epidermal layer (e.g., an artificial epidermal layer). In some embodiments, an epidermal layer can be an engineered epidermis equivalent, e.g., an artificial epidermal layer formed in vitro.

In some embodiments, an epidermal layer can comprise one or more types of cells, including keratinocytes, melanocytes, Langerhans cells, Merkel cells, and inflammatory cells. In some embodiments, an epidermal layer can comprise keratinocytes. In some embodiments, keratinocytes in an epidermal layer can include epithelial keratinocytes, basal keratinocytes, proliferating basal keratinocytes, differentiated suprabasal keratinocytes, or any combination thereof.

In some embodiments, an epidermal layer can comprise an engineered cell. In some embodiments, an epidermal layer can comprise an immortalized cell. In some embodiments, an epidermal layer can comprise at least basal keratinocytes, e.g., keratinocytes which may not be differentiated. In some embodiments, an epidermal layer can further comprise partially differentiated keratinocytes as well as fully differentiated keratinocytes. In some embodiments, one or more epidermal layers in a cruelty-free leather can be a transition from undifferentiated basal keratinocytes to fully differentiated keratinocytes as one progresses from a dermal-epidermal junction where a basal keratinocyte can be localized.

In some embodiments, basal keratinocytes can express hemidesmosomes, which can serve to help secure an epidermal and a dermal layer together. In some embodiments, basal keratinocytes can also serve to regenerate a skin. In some embodiments, an epidermal layer in a cruelty-free leather herein can have basal keratinocytes that serve these functions. In some embodiments, a cruelty-free leather comprising such basal keratinocytes can be capable of regeneration. In some embodiments, distinctions between basal keratinocytes and differentiated keratinocytes in one or more epidermal layers in a cruelty-free leather can be that both E- and P-cadherin's can be present in epidermal keratinocytes along a basal membrane zone (BMZ), but keratinocytes which may be differentiated and located away from a BMZ may only express E-cadherin.

In some embodiments, a basal keratinocyte of an epidermal layer can be aligned in a layer in direct contact with a dermal layer, serving as a boundary between a differentiated keratinocyte and a fibroblast. In alternative cases, there can be gaps between a basal keratinocyte and a dermal layer. Still further, there can be gaps between a basal keratinocyte and other basal keratinocytes, leaving gaps between a differentiated keratinocyte and a dermal layer. In these latter cases where there can be gaps between a basal or differentiated keratinocyte and a dermal layer, a dermal and epidermal layer may not be uniformly in contact with one another but can be adjacent to each other. In some embodiments, a dermal and an epidermal layer can be adjacent in that there can be generally fluid, but substantially no other intervening materials such as layers of cells, collagen, matrices or other supports between a dermal and an epidermal layer.

In some embodiments, keratinocytes in an epidermal layer can express one or more markers. In some embodiments, markers can include, but may not be limited to, Keratin 14 (KRT14), tumor protein p63 (p63), Desmoglein 3 (DSG3), Integrin, beta 4 (ITGB4), Laminin, alpha 5 (LAMA5), Keratin 5 (KRT5), an isoform of tumor protein p63 (e.g., TAp63), Laminin, beta 3 (LAMB3), and Keratin 18 (KRT18).

In some embodiments, a thickness of an epidermal layer can be engineered to fit a function or use of a cruelty-free leather. In some embodiments, an epidermal layer can have a thickness from about 0.001 mm to about 10 mm. In some embodiments, an epidermal layer can have a thickness from about 0.005 mm to about 10 mm, from about 0.005 mm to about 5 mm, from about 0.005 mm to about 2 mm, from about 0.01 mm to about 10 mm, from about 0.01 mm to about 5 mm, from about 0.01 mm to about 2 mm, from about 0.01 mm to about 1, from about 0.01 mm to about 0.8 mm, from about 0.01 mm to about 0.4 mm, from about 0.01 mm to about 0.2 mm, from about 0.01 mm to about 0.1 mm, from about 0.05 mm to about 0.4 mm, from about 0.05 mm to about 0.2 mm, from about 0.05 mm to about 0.1 mm, from about 0.1 mm to about 0.4 mm, from about 0.1 mm to about 0.2 mm, from about 0.08 mm to about 1 mm, or from about 0.05 mm to about 1.5 mm. In some embodiments, an epidermal layer can have a thickness from about 0.01 mm to about 2 mm. In some embodiments, an epidermal layer can have a thickness from about 0.1 mm to about 0.22 mm. In some embodiments, a thickness of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a thickness of a dermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, thickness values described herein can be a thickness of an epidermal layer and a basement membrane substitute.

In some embodiments, a length of an epidermal layer can be engineered to fit a function or use of a cruelty-free leather. In some embodiments, an epidermal layer can have a length from about 0.01 mm to about 50 m. In some embodiments, an epidermal layer can have a length from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, an epidermal layer can have a length from about 0.02 mm to 5 mm. In some embodiments, an epidermal layer can have a length from about 0.1 mm to 0.5 mm. In some embodiments, an epidermal layer can have a length from about 0.2 mm to 0.5 mm. In some embodiments, a length of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a length of an epidermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, an epidermal layer can have a length of at least about 50, 60, 70, 80, 90, 100, 200, 300, 400, 500, 700, 1000 mm. In some embodiments, an epidermal layer can have a length of at least about 50, 60, 70, 80, 90, 100, 200, 300, 400, 500, 600, 700 cm. In some embodiments, an epidermal layer can have a length of at least about 50, 60, 70, 80, 90, 100, 200, 300, 400 m.

In some embodiments, a width of an epidermal layer can be engineered to fit a function or use of a cruelty-free leather. In some embodiments, an epidermal layer can have a width from about 0.01 mm to about 50 m. In some embodiments, an epidermal layer can have a width from about 0.01 mm to about 10 mm, from about 0.01 mm to about 8 mm, from about 0.01 to about 5 mm, from about 0.02 to about 5 mm, from about 0.05 to about 5 mm, from about 0.1 to about 5 mm, from about 0.1 to about 2 mm, from about 0.1 to about 1 mm, from about 0.1 to about 0.8 mm, or from about 0.1 to about 0.5 mm. In some embodiments, an epidermal layer can have a width from about 0.02 mm to 5 mm. In some embodiments, an epidermal layer can have a width from about 0.1 mm to 0.5 mm. In some embodiments, an epidermal layer can have a width from about 0.2 mm to 0.5 mm. In some embodiments, a width of an epidermal layer can be at least 0.001 mm, 0.01 mm, 0.02 mm, 0.04 mm, 0.08 mm, 0.1 mm, 0.2 mm, 0.4 mm, 0.8 mm, 1 mm, 2 mm, 4 mm, 8 mm, or 10 mm. In some embodiments, a width of an epidermal layer can be at most 50 mm, 40 mm, 20 mm, 10 mm, 8 mm, 4 mm, 2 mm, 1 mm, 0.8 mm, 0.4 mm, 0.2 mm, 0.1 mm, 0.08 mm, 0.04 mm, 0.02 mm, or 0.01 mm. In some embodiments, an epidermal layer can have a width of at least about 50, 60, 70, 80, 90, 100, 200, 300, 400, 500, 700, 1000 mm. In some embodiments, an epidermal layer can have a width of at least about 50, 60, 70, 80, 90, 100, 200, 300, 400, 500, 600, 700 cm. In some embodiments, an epidermal layer can have a width of at least about 50, 60, 70, 80, 90, 100, 200, 300, 400 m.

In some embodiments, an epidermal layer can be stratified, e.g., having a plurality of sublayers. In some embodiments, sublayers can have different cell compositions, e.g., different types of keratinocytes. In some embodiments, sublayers can comprise engineered cells. In some embodiments, sublayers of an epidermal layer can have different thicknesses and/or densities. In some embodiments, an epidermal layer can have one or more of cornified layer (Stratum corneum), clear/translucent layer (Stratum lucidum), granular layer (Stratum granulosum), spinous layer (Stratum spinosum), basal/germinal layer (Stratum basale/germinativum), or any combination thereof. In some embodiments, an epidermal layer can comprise functional epidermal permeability barrier (e.g., organized lipid bilayers in Stratum corneum). In some embodiments, a Stratum corneum, Stratum lucidum, stratum granulosum, Stratum spinosum, or Stratum basale/germinativum, can have a thickness of about 0.0001 mm to about 5 mm. In some embodiments, a Stratum corneum, Stratum lucidum, stratum granulosum, Stratum spinosum, or Stratum basale/germinativum, can have a thickness of at least about 0.001 mm, 0.01 mm, 0.02 mm, 0.04 mm, 0.08 mm, 0.1 mm, 0.15 mm, 0.2 mm, 0.4 mm, 0.8 mm, 1 mm, 2 mm, 4 mm, 8 mm, or 10 mm. In some embodiments, a Stratum corneum, Stratum lucidum, stratum granulosum, Stratum spinosum, or Stratum basale/germinativum, can have a thickness of at most about 50 mm, 40 mm, 20 mm, 10 mm, 8 mm, 4 mm, 2 mm, 1 mm, 0.8 mm, 0.4 mm, 0.2 mm, 0.15 mm, 0.1 mm, 0.08 mm, 0.04 mm, 0.02 mm, or 0.01 mm.

In some embodiments, an epidermal layer can further comprise cells producing pigments, e.g., melanin. In some embodiments, such pigment-producing cells can be melanocytes. In some embodiments, melanocytes in an epidermal layer can express one or more markers. In some embodiments, such markers can include, but may not be limited to, SRY-box containing gene 10 (Sox-10), Microphthalmia-associated transcription factor (MITF-M), premelanosome protein (gp-100), Dopachrome tautomerase (DCT), Tyrosinase (TYR), and Melan-A (MLANA). In some embodiments, a cruelty-free leather may not comprise an epidermal layer.

Provided herein are methods of making any one of the compositions provided herein. In some embodiments, the method comprises at least partially coating the scaffold with a coating comprising a Matrigel, a vitronectin, a fibronectin, a protein extract from a soy, a protein extract from a pea, a protein extract from a corn, a peptide generated from synthesis, an RNA-binding glycine-rich (RBG) protein, a synthetic protein, an RGD peptide, a polylysine, a polyarginine, a polyornithine, a recombinant protein, an oligomer, a polymer, GTAMC, carbohydrate-binding module, a cellulose binding domain, a starch binding domain, or a combination thereof. In some embodiments, the method further comprises seeding an isolated animal fibroblast or fibroblast-like cell onto the scaffold to form the composition. In some embodiments, the collagen, fibroblast, and/or fibroblast-like cell is associated with the scaffold by any of the means as described elsewhere herein, such as non-specific adsorption, covalent interactions, or specific adsorption.

In some embodiments, a method of making a cruelty-free leather can comprise forming and tanning a cell and scaffold composition. In some embodiments, a method can comprise further processing a cell and scaffold composition, e.g., to achieve natural leather thickness and texture. In some embodiments, a tanning can be performed after an at least partial decellularization of a cell layer. In some embodiments, a tanning can make a cruelty-free leather resemble a natural leather, which can be a durable and flexible material created by a tanning of animal rawhide and skin, often cattle hide. Tanning herein can refer to a process of treating skins of animals to produce leather or treating compositions disclosed herein to produce cruelty-free leather. Tanning can be performed various ways, including vegetable tanning (e.g., using tannin), chrome tanning (chromium salts including chromium sulfate), aldehyde tanning (using glutaraldehyde or oxazolidine compounds), syntans (synthetic tannins, using aromatic polymers), bacterial dyeing, and the like. In some embodiments, tanning can be metal-free. In some embodiments, tanning can be an environmentally friendly process. In some embodiments, tanning can be performed to convert proteins in a hide, skin, or composition disclosed herein into a stable material that will not putrefy, while allowing the material to remain flexible. In some embodiments, chromium can be used as tanning material. In some embodiments, tanning can comprise a chromium, an aluminum, a zirconium, a titanium, an iron, a sodium aluminum silicate, a formaldehyde, a glutaraldehyde, an oxazolidine, an isocyanate, a carbodiimide, a polycarbamoyl sulfate, a tetrakis hydroxyphosphonium sulfate, a sodium p-[(4,6-dichloro-1,3,5-triazin-2-yl)amino]benzenesulphonate, a pyrogallol, a catechol, a syntan or any combination thereof. In some embodiments, tanning can be performed on engineered cells in a scaffold. In some embodiments, a tissue that can be tanned can comprise fibers or a plurality of fibers (e.g., polyester fibers, synthetic fibers, natural fibers). In some embodiments, a pH of a cell layer or layered structure can be adjusted (e.g., lowered; e.g., to pH about 2.8-3.2) to enhance a tanning. In some embodiments, following tanning a pH can be raised (“basification” to a slightly higher level, e.g., pH about 3.8-4.2). In some embodiments a pH described herein can be at least 1. In some embodiments, a pH described herein can be 14 or less. In some embodiments, tanning can be performed on cell layers, e.g., dermal layers, epidermal layers, engineered cells, immortalized cell layers, laminin, fibronectin, collagen or any combination thereof. In some embodiments, tanning can be performed on a tissue (e.g., a tissue from an engineered cell). In some embodiments, tanning can also be performed on layered structures, e.g., layered structures comprising at least a dermal layer. In certain cases, tanning can be also performed on a synthesized leather. In some embodiments, tanning can be performed after forming cell layers, e.g., dermal layers or epidermal layers. In some embodiments, tanning can be performed after forming layered structures.

In some embodiments, the methods provided herein may further comprise processing. Processing may take place on the one or more cell layers, such as one or more cell layers on a scaffold. (Further) processing may take place on a (e.g., tanned) hide. In some embodiments, processing can be selected from a group consisting of preserving, soaking, bating, pickling, depickling, thinning, retanning, lubricating, crusting, wetting, sammying, shaving, rechroming, neutralizing, dyeing, fatliquoring, filling, stripping, stuffing, whitening, fixating, setting, drying, conditioning, milling, staking, buffing, finishing, oiling, brushing, padding, impregnating, spraying, roller coating, curtain coating, polishing, plating, embossing, ironing, glazing, tumbling, and any combination thereof.

In some embodiments, a cruelty-free leather can comprise collagen and extracellular matrix components produced by cells in a dermal layer and/or an epidermal layer disclosed herein. In some embodiments, a cruelty-free leather can comprise an at least partially decellularized dermal layer and/or an epidermal layer, as disclosed herein. In some embodiments, a cruelty-free leather does not comprise an epidermal layer. In some embodiments, a cruelty-free leather also can comprise at least a portion of hair follicle cells, endothelial cells, smooth muscle cells, dermal papilla cells, immune system cells (such as lymphocytes, dendritic cells, mast cells, macrophages or Langerhans cells), adipocytes, nerve cells, Schwann cells, and a mixture thereof. In some embodiments, a cruelty-free leather can comprise at least a portion of an engineered cell (e.g., a cell comprising a molecular switch). A cruelty-free leather can comprise an immortalized cell. In some embodiments, a cruelty-free leather can comprise an isolated cell. In some embodiments, a cruelty-free leather can comprise a cell line.

In some embodiments, a cruelty-free leather can comprise a hair. In some embodiments, a cruelty-free leather can comprise a hair in one or more layered structures. In some embodiments, a cruelty-free leather can comprise a fur. In some embodiments, a hair (e.g., fur) can be natural, synthetic, or a combination thereof. In some embodiments, a hair (e.g., fur) can be grown from cells in a cruelty-free leather or added to a cruelty-free leather from an exogenous source. In some embodiments, a cruelty-free leather may not have any hair.

A cruelty-free leather can comprise at least a portion of prokaryotic cells, eukaryotic cells or a combination thereof. In some embodiments, a cruelty-free leather can comprise at least a portion of a bacterium cell, for example, Escherichia coli. In some embodiments, a cruelty-free leather can comprise at least a portion of a eukaryotic cell (e.g., a bovine cell, a porcine cell, a human cell, Saccharomyces cerevisiae).

In some embodiments, at least a portion of one or more cells in a cruelty-free leather can be genetically engineered cells. The term “genetically engineered” can refer to a man-made alteration to a nucleic acid content of a cell. Therefore, genetically engineered cells can include cells containing an insertion, deletion, and/or substitution of one or more nucleotides in a genome of a cell as well as alterations including an introduction of self-replicating extrachromosomal nucleic acids inserted into a cell. Genetically engineered cells also include those in which transcription of one or more genes has been altered, e.g., increased or reduced.

In some embodiments, a thickness of leather units can be reported in millimeters, ounces, or irons. In some embodiments, one ounce can equal 1/64 in. or 0.0156 in. or 0.396 mm. In some embodiments, one iron can equal ¼s in. or 0.0208 in. or 0.53 mm.

### Methods of Use

Disclosed herein in some embodiments, is a method comprising transplanting a composition disclosed herein onto a patient in need of a skin graft for a treatment of lost or damaged skin. In some embodiments, a patient can have lacerations, contusions, lesions, sores, burns, wounds, surgical wounds, surgically removed skin, necrotic skin, or any combination thereof. Disclosed herein in some embodiments, is a method comprising tanning a composition disclosed herein to produce a cruelty free leather. Disclosed herein in some embodiments, is a method of using a cruelty-free leather disclosed herein as a substitute for a traditional leather in a leather article. Disclosed herein is a leather article comprising a cruelty-free leather as disclosed herein. In some embodiments, a leather article can comprise a watch strap, a belt, a suspender, a packaging, a shoe, a boot, a footwear, a glove, a clothing, a bag, a clutch, a purse, a coin purse, a billfold, a key pouch, a credit card case, a pen case, a backpack, a case, a wallet, a saddle, a harness, a whip, a luggage, a travel good, a rucksack, a portfolio, a document bag, a briefcase, an attache case, a pet article, a leash, a collar, a hunting and fishing article, a gun case, a cutlery case, a holster for a fire arm, a stationary article, a writing pad, a book cover, a camera case, a spectacle case, a cigarette case, a cigar case, a jewel case, a mobile phone holster, a sport article, a ball, a basketball, a soccer ball, a football, or any combination thereof. In some embodiments, a clothing can comprise a top, a bottom, an outerwear, or any combination thereof. In some embodiments, a bag can comprise a handbag with or without shoulder strap. In some embodiments, a luggage can comprise a trunk, a suitcase, a travel bag, a beauty case, a toilet kit, or any combination thereof. Disclosed herein in some embodiments, is a method of using a composition as disclosed herein for a treatment of a disease or ailment. Disclosed herein in some embodiments, is a kit comprising a composition as disclosed herein or a leather article as disclosed herein.

### EXAMPLES

### Example 1. Cell Attachment on Nylon and Bamboo/Cotton Scaffolds

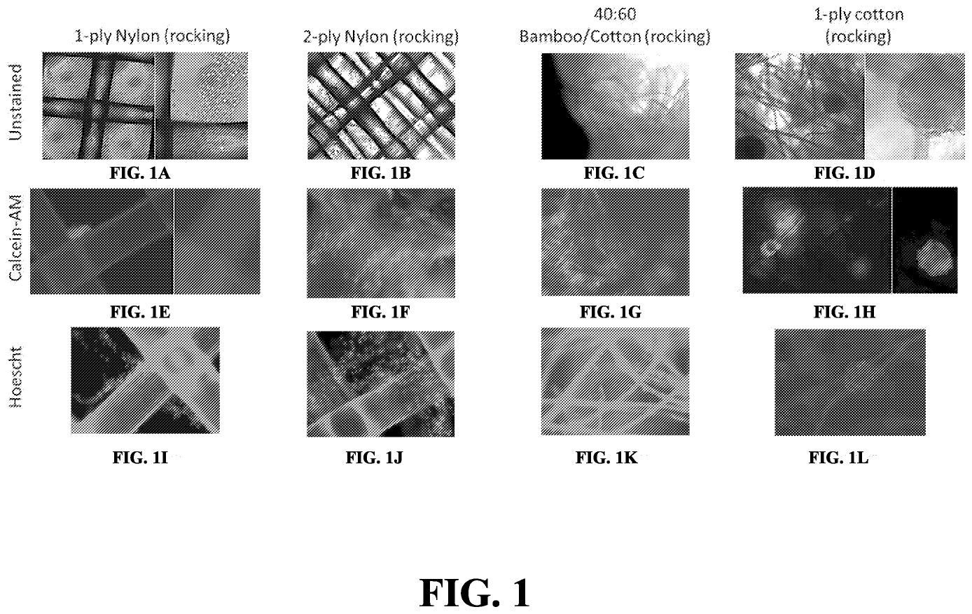

Bovine dermal fibroblasts were attached to 1- or 2-ply nylon scaffolds or 40:60 bamboo/cotton scaffolds over a duration of 17 days in static and rocking growth conditions in order to evaluate cell attachment on the various scaffold materials. Cell growth was assessed via measurement of the remaining glucose level before each feed. The rocking conditions, in general, resulted in the consumption of more glucose. The bamboo/cotton blend consumed the most glucose in both static and rocking conditions and the rocking 2-ply nylon glucose consumption steadily increased over time. Microscopy including on cells stained with Calcein-AM and Hoescht stains seen in FIG. 1A, FIG. 1B, FIG. 1C, FIG. 1E, FIG. 1F, FIG. 1G, FIG. 1I, and FIG. 1J indicated that cell attachment on the 1-ply nylon surface was lower than that on the 2-ply nylon surface in which clear extracellular matrix formation is evident on the scaffold. The microscopy as seen in FIG. 1D, FIG. 1H, and FIG. 1K indicated the best growth and spindle-like cells in both rocking and static conditions for cells attached to the bamboo/cotton scaffolds. These data indicated by that of the nylon indicate that woven materials may not provide sufficient surface area or tortuosity for good cell attachment. Alternatively, non-woven materials such as that of the bamboo/cotton blend exhibit significantly improved cell attachment and non-woven nylons such as needle-punch nylon scaffolds exhibit increased cell attachment in comparison to simple mesh furthering the benefit of non-woven materials.

### Example 2. Conditions for Dissolving of Scaffolds

Poly(vinyl alcohol) (PVOH) and poly(lactic acid) (PLA) were both tested as dissolvable scaffolds for fibroblast cell attachment to prevent fiber protrusion and improve tissue quality. The conditions required to dissolve the PVOH scaffold were deionized water or DMEM at 95° C. for 1 minute. The conditions required to dissolve PLA require the use of organic solvents such as benzylamine or ethyl acetate overnight. The success of dissolving the scaffold was determined by observation of visual changes to assess removal of the scaffold and potential hide damage as well as weight of scaffolds pre- and post-dissolution as seen in Table 1. Dissolution of PLA in benzylamine overnight resulted in a 72% reduction of the scaffold by weight whereas dissolution in ethyl acetate under similar conditions resulted in 93% loss of the scaffold by weight. The effects of the dissolution conditions on the hide were assessed on raw salted hide (pre-tan) as well as tanned but not fat liquored hide (post-tan).

Exposing the pre-tanned hides on PVOH scaffolds to high temperatures (95° C.) destroys the tissue. However, exposing the tanned hides on PVOH scaffolds to high temperatures (95°) does not result in complete destruction but does result in reduction in hide quality while resulting in ˜100% removal of the scaffold. Lower dissolution temperatures (90° C.) in water for longer periods of time including 45 minutes or 1.5 hours resulted in 27.3% and 46.7% removal of the scaffold respectively but result in damage to the hide since the denaturation temperature for the tanned hide is ˜70° C. Due to the lower denaturation temperature, a lower dissolution temperature of 40-50° C. may be preferable to avoid hide damage. However, exposure to benzylamine and ethyl acetate result in no major changes to the hides on PLA scaffolds pre- and post-tanning. This data highlights the ability to dissolve the PLA and PVOH scaffold in various conditions in order to aid in preventing fiber protrusion through the tissue and improve tissue quality.

TABLE 1

| Condition | Pre-weight (mg) | Post Weight (mg) | % Reduction |
|---|---|---|---|
| Benzylamine, | 85.1 | 23.7 | 72% |
| overnight | | | |
| Ethyl acetate, | 82.6 | 5.7 | 93% |
| overnight | | | |
| Deionized Water, | Not measured | Not measured | ~100% |
| 95° C., 1 minute | | | |
| DMEM, 95° C., | Not measured | Not measured | ~100% |
| 1 minute | | | |
| 90° C., 45 minutes | 51.9 | 37.7 | 27.3% |
| 90° C., 1.5 hours | 59.7 | 31.8 | 46.7% |

### Example 3. Effects of Scaffold Pore Size on Tissue Quality

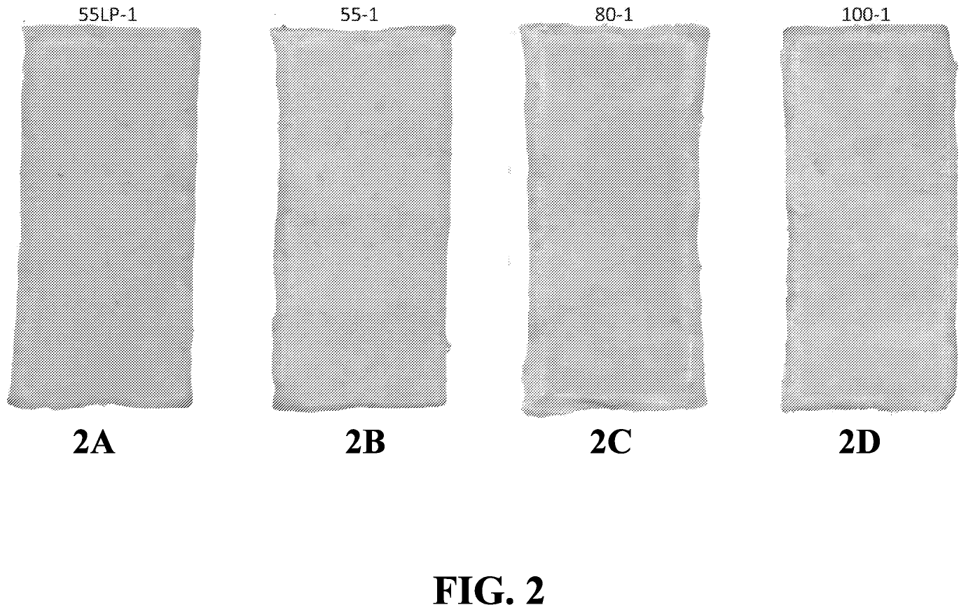

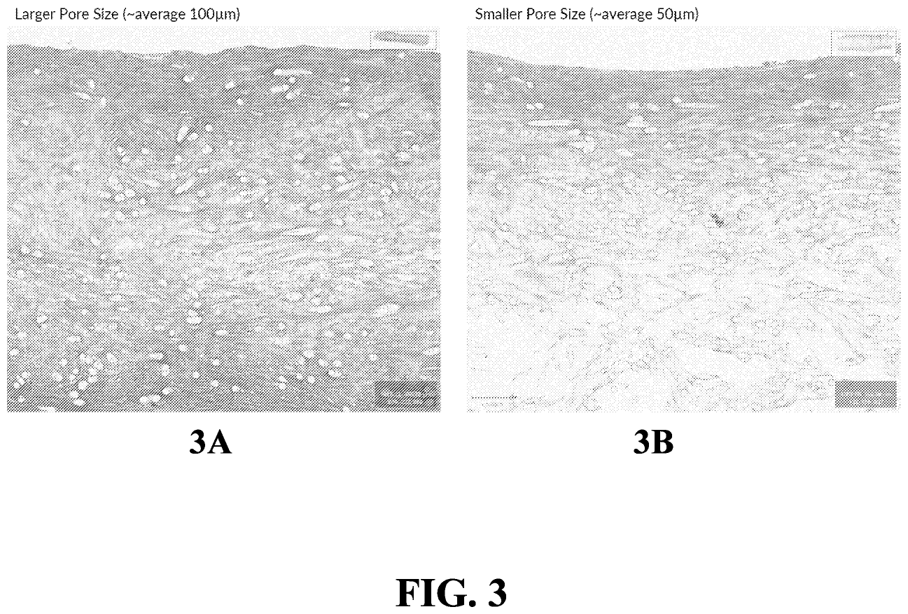

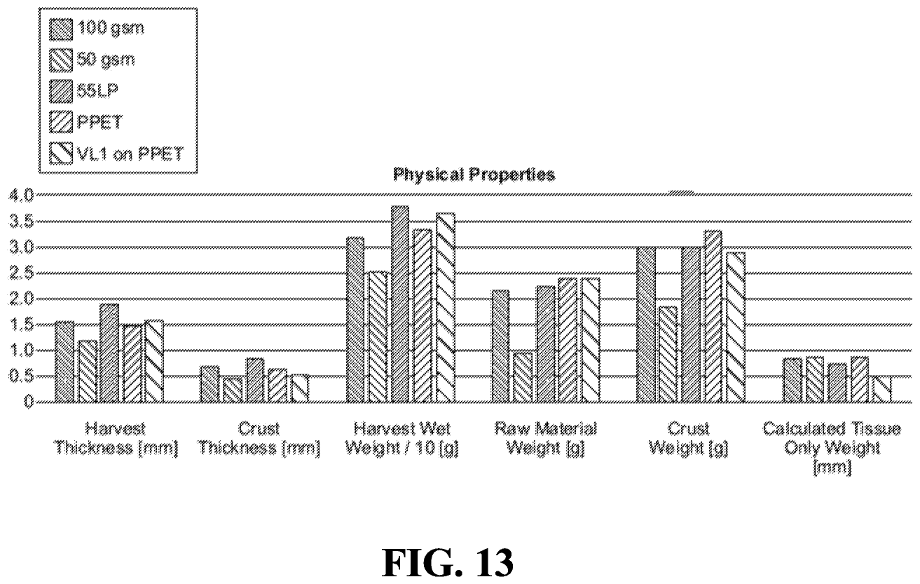

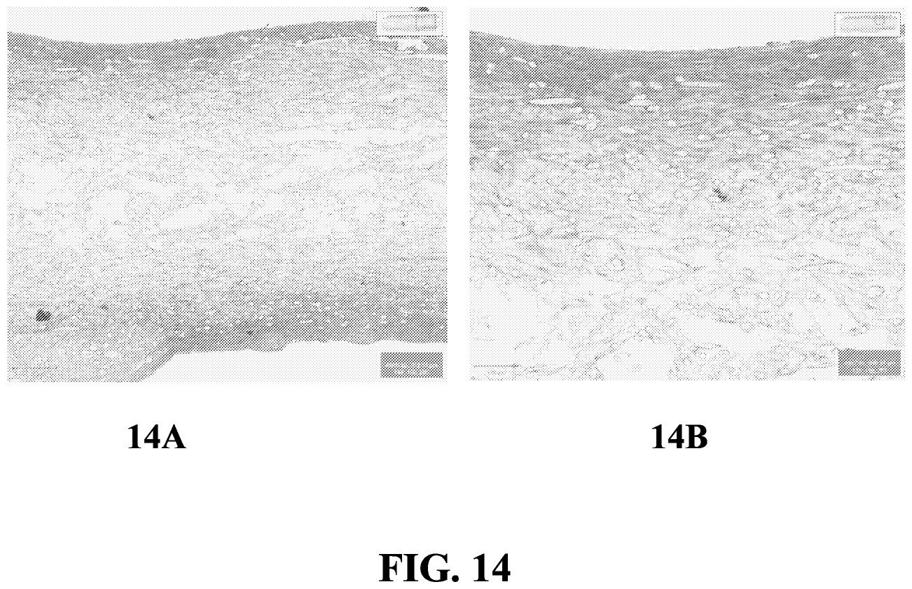

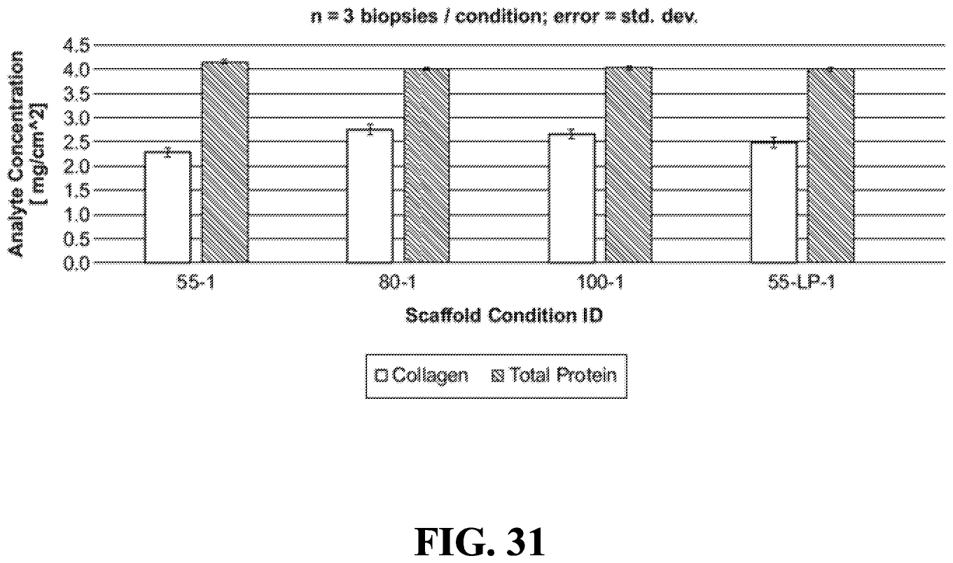

A series of PLA scaffold materials were tested with varying pore sizes including 55 μm, light punch 55 μm (55LP), 80 μm, and 100 μm and tissue quality was assessed as a function of pore size in a cardholder format. Scaffolds were assembled onto frames and placed into bioreactors. A seeding solution (2.75×10⁶ cells/mL) was used to seed the scaffolds and allowed to remain static while rocking conditions were started after 1 week. There was no feeding 2 days after seeding, thereafter cells were fed 7 days per week with full media exchange and the culture media was sampled every day. Tissues were allowed to grow for 27 days, and the tissue biopsies were taken using sterile 4 mm biopsy punches. No significant differences were observed in sulfated glycosaminoglycans (sGAG) after 24 or 96 hours (n=2 biopsies) nor were there any significant differences in collagen and total protein concentration between scaffolds of varying pore sizes (n=3 biopsies) (FIG. 31). The 55LP scaffold exhibited the highest bending stiffness of raw material and raw hide. This indicated that the increased thickness and thus moment of inertia translates to an increase in stiffness of the material. Scaffolds with smaller pore sizes were heavier both prior and following culture, resulting in a tanned hide that felt more robust as seen in (FIG. 13). However, the calculated net weight of tissue content across all scaffolds was similar, regardless of pore size as seen in FIG. 13. Histology was completed on tissue cross sections stained with trichrome. Larger pore sizes generally lead to better tissue ingrowth as seen in FIG. 15A-F where the average pore size is 100 μm in comparison to FIG. 16A-F where the average pore size is 50 μm. The 55LP scaffolds, which had the smallest pore size and largest starting thickness were observed to have the worse degree of tissue ingrowth as seen in FIG. 14A-F. In general, larger pore size materials showed more inconsistencies and light spots upon scanning as seen in FIG. 2A, FIG. 2B, FIG. 2C, and FIG. 2D but in general, the PLA scaffolds resulted in increased consistency in hides over pressed PET scaffolds. The calculated weight of tissue across all conditions was similar, regardless of pore size. Histology was completed on tissue cross sections stained with trichrome. Larger pore sizes generally lead to better tissue ingrowth as seen in FIG. 3A where the porosity is 100 μm in comparison to FIG. 3B where the porosity is 50 μm. Scaffolds with larger pores provide better tissue ingrowth whereas smaller pores sizes lead to more weight contribution from the fibers creating the illusion of a weightier, stronger hide indicative of the importance of the porosity of the scaffold material.

### Example 4. Impact of Soaking PLA

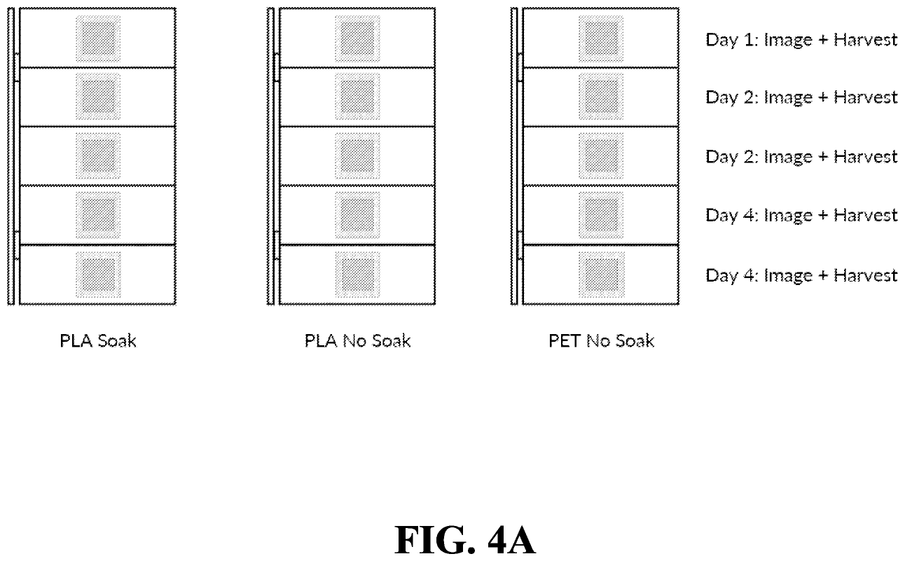

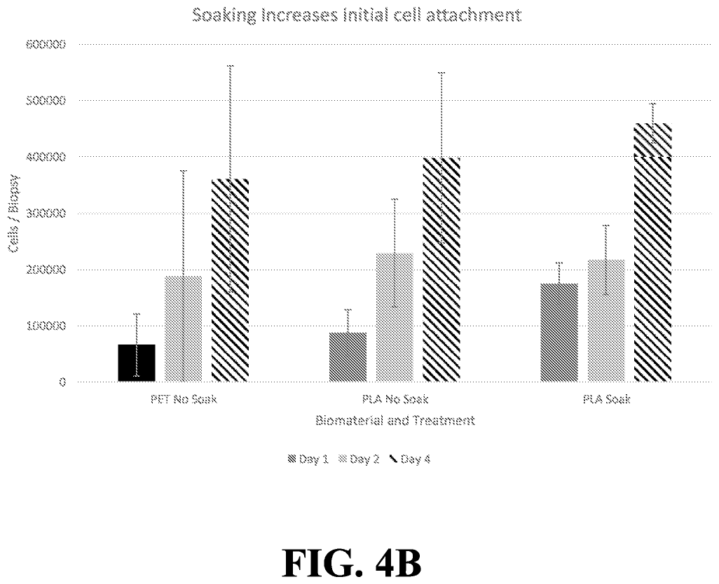

The PLA scaffold was tested to determine if soaking in fetal bovine serum (FBS) is required for adequate cell attachment and proliferation. The soaking in FBS allows for the adsorption of sticky proteins to the surface thus allowing for increased cell attachment. Two boxes of 5 square scaffolds made of 1.7 dtex PLA and 60 μm pore size were prepared with one control box containing PET. One set of the 5 scaffolds was filled with pure FBS as to cover the channels and soaked overnight at 37° C., while the second set and the control (PET scaffolds) were not (see FIG. 4A). After aspiration of the FBS, all channels were seeded with fibroblast cells. These scaffolds were harvested at variable times. These scaffolds were biopsied 1× on day 1, 2× on day 2, and 2× on day 4 using a sterile biopsy punch and media cells were saved for analysis. The biopsies from days 2 and 4 were used for further analysis. Three of these biopsies were used for DNA assays and one of which was used for Calcein imaging. The DNA assays indicated that FBS soaked PLA scaffolds result in the highest initial attachment of cells in comparison to unsoaked PLA scaffolds (FIG. 4B), thus soaking of the channels and the adsorption of proteins to the surface has a beneficial effect.

### Example 5. Protein-Scaffold Conjugation for Cell Attachment and Proliferation

Poor attachment of cells to biomaterials after seeding can result in cell death, reduced viability, and poor collagen production which can be addressed by increasing the cytocompatibility of biomaterial surfaces. As observed in Example 2, traditional cell culture proteins may be applied to increase cell attachment. Lower cost, cruelty free options were also evaluated. Proteins extracted from sweet pea and wheat flour were conjugated to PLA and Lyocell and compared to the respective unmodified scaffold materials to determine whether functionalization of scaffold surfaces could enhance cell attachment and proliferation. Proteins were conjugated to PLA using carbodiimide crosslinker chemistry (EDC/NHS) via linkages between the activated carboxylic acids of the PLA and amines of the protein. Attempts to enhance the conjugation of protein to PLA included the addition of carboxyl groups to PLA before undergoing conjugation with the protein. Protein was conjugated to Lyocell via a Maillard reaction in an autoclave. Autoclaving was completed once, twice, or four times. Cell culture conditions were as follows: 500 k/cm² in tissue culture treated 48 well plate using DMEM HG with 10% FBS. Cell attachment and proliferation were measured via MTT and DNA assays which indicated that protein conjugation to Lyocell but not PLA significantly enhanced cell attachment and proliferation in comparison to their unmodified counterparts. FIG. 5 shows the percentage of cells on the pea protein, wheat protein, and non-treated (NT) scaffolds after 1 and 4 days highlighting the increase exhibited upon modification of the Lyocell surfaces. Fluorescence images after 1 day as seen in FIG. 6D indicated that in unmodified Lyocell, aggregation of cells is occurring whereas after conjugation with pea protein (as seen in FIG. 6E), spindle-like growth is occurring highlighting the increased cytocompatibility post-modification. The conjugation of pea or wheat protein to PLA results in little change in the spindle-like cell growth seen in the microscopy of FIG. 6A, FIG. 6B, and FIG. 6C. Furthermore, conjugation of sweet pea protein was shown to be more effective than wheat flour protein (as seen in FIG. 5, FIG. 6B, FIG. 6C, FIG. 6E, and FIG. 6F). These examples are indicative of the potential for increasing cytocompatibility of scaffold surfaces by treating them with groups of increased biocompatibility such as these or others including polypeptides. Furthermore, chemical processing to adjust the surface properties has the potential to increase cell affinity or tissue outcomes which can be achieved via a variety of synthetic approaches such as through treatment with sodium hydroxide or glycidyl trimethylammonium chloride (GTMAC).

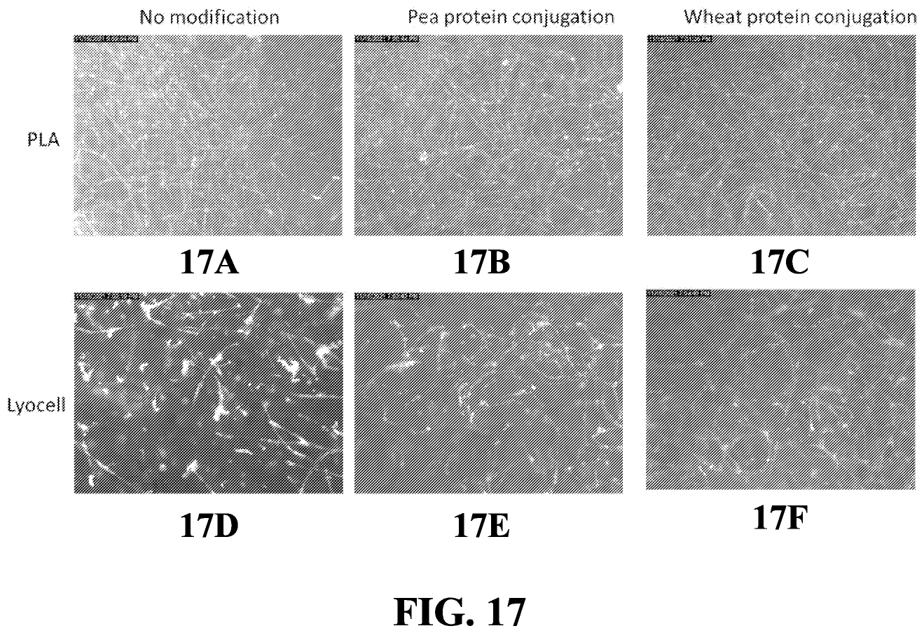

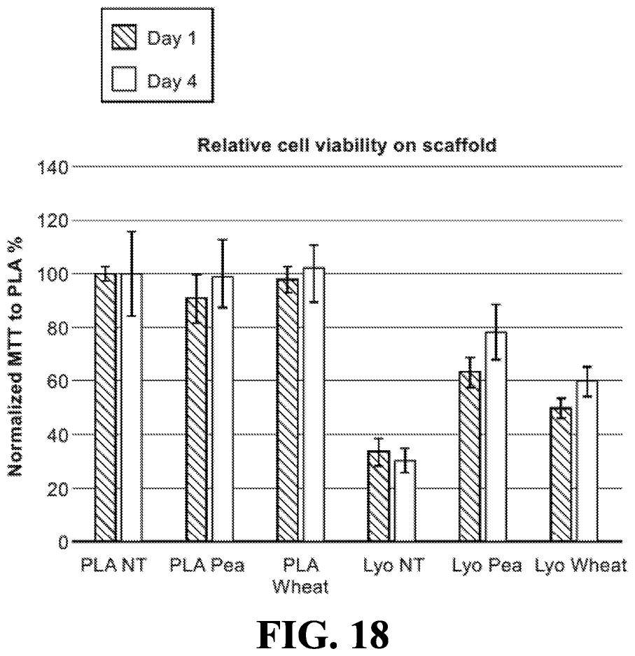

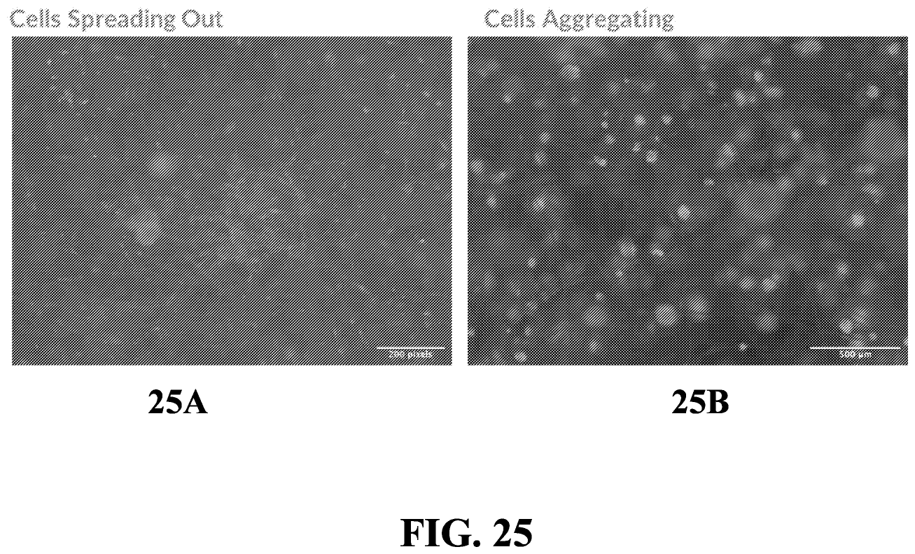

In another example, pea and wheat proteins were first extracted and sterilized. Pea and wheat proteins were incubated with PLA and Lyocell based scaffolds. Cells were then seeding on biopsy punches of these scaffolds and cultured in well plates. The scaffolds were imaged with Calcein AM, and cell numbers and activity were measured with DNA and MTT assays respectively. An example can be seen of the difference between spread out cells and cells aggregating and rounding up in FIGS. 25A-B respectively, where the former is the preferred morphology denoting the affinity of a cell for the substrate. No changes in cell morphology as seen in FIGS. 17A-C, or cell activity, as seen in FIG. 18, were found for PLA. However, there was an 80% increase in cell activity with the Pea protein treatment on lyocell and a 40% increase with wheat protein as seen in FIG. 18, as well as a shift in cell morphology from rounded to spindle like, demonstrating an increase in affinity for the surface with treatment, as seen FIGS. 17D-F.

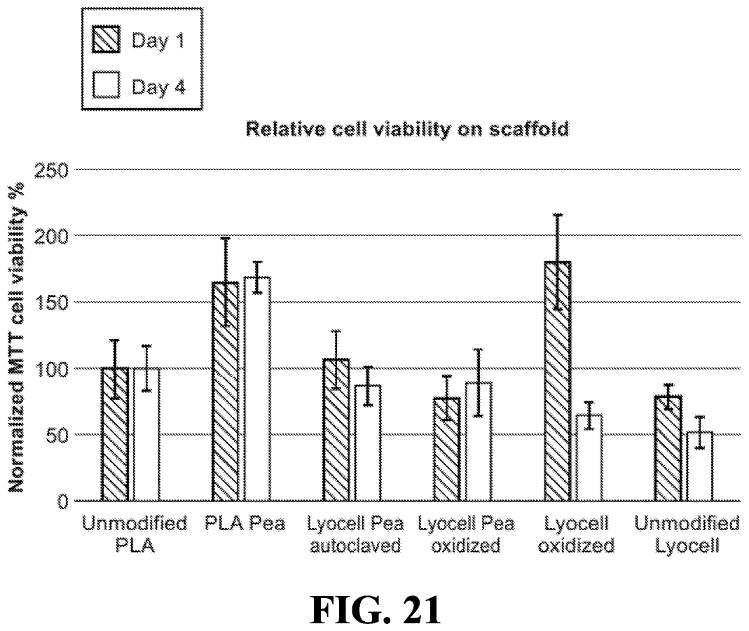

Additional conjugation methods were tested to see if the utilization of these low-cost proteins could be improved, including the Maillard reaction, surface activation, and EDC/NHS conjugation. The Maillard reaction was approximated by autoclaving the scaffold while soaked in a protein rich solution. In some cases, the scaffold surface was activated by oxidation with sodium periodate, and then the scaffolds were either seeded as is, or proteins were then conjugated via the Maillard reaction. Proteins were conjugated to PLA using carbodiimide crosslinker chemistry (EDC/NHS) via linkages between the activated carboxylic acids of the PLA and amines of the protein. The Maillard reaction was found to assist in pea protein conjugation to Lyocell as seen in FIG. 20C with the improvement in cell spreading compared to the unmodified scaffold as seen in FIG. 20B. Use of EDC/NHS chemistry to covalently bond Pea proteins to PLA was also found to increase Cell activity by over 50% compared to unmodified PLA, as seen in FIG. 21, where previous cold coating was found to make no change in cell activity, as seen in FIG. 18. Oxidation of Lyocell on its own did not result in long lived cell attachment. While on the first day after seeding it can be seen that cell activity spiked greatly, however, this activity greatly fell off by day 4, as seen in FIG. 21, and this reduction of cell activity can be corroborated by the lack of spread-out cells in the calcein images taken on day 4 as seen in FIG. 20E. However, a combination of both oxidation and the Maillard reaction with Pea protein on Lyocell resulted in cells that appeared to be very well spread out as seen in FIG. 20F, however this increase in cell spreading was not reflected in the cell activity numbers seen in FIG. 21.

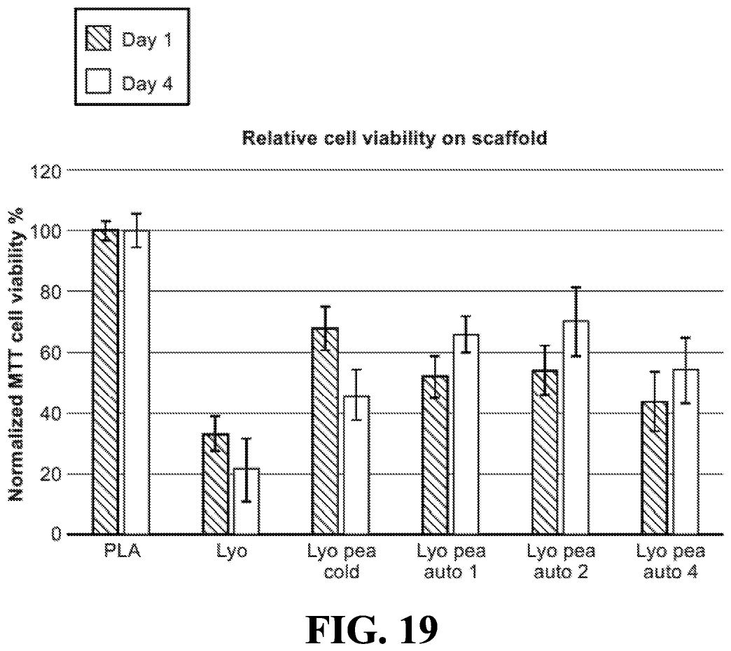

The Maillard reaction was further investigated by adjusting the number of autoclave cycles. A dry cycle on the autoclave was completed once, twice, or four times which was compared against soaking purely in a cold solution without any autoclave cycles. As seen in FIG. 19, one and two autoclave cycles resulted in the highest cell activity on day 4 after seeding, with over 3× the activity compared to the untreated condition. Cold soaking was found to highly increase cell activity at day 1, however, this activity greatly decreased by day 4. This decrease in activity could be due to protein groups detaching from the scaffold thus decreasing activity. The initial increase in activity compared to one, two, or four autoclave cycles could be due to a higher initial quality of proteins which have been through fewer denaturing autoclave cycles.

These examples of activating scaffold surfaces and conjugating protein sources are indicative of the potential for increasing cytocompatibility of scaffold surfaces by treating them. Furthermore, chemical processing to adjust the surface properties has the potential to increase cell affinity or tissue outcomes. While not shown here, other methods which could be used to activate the surface of the fibers include treatment with sodium hydroxide or glycidyl trimethylammonium chloride (GTMAC).

### Example 6. Seeding Density Effects on Tissue Outcome

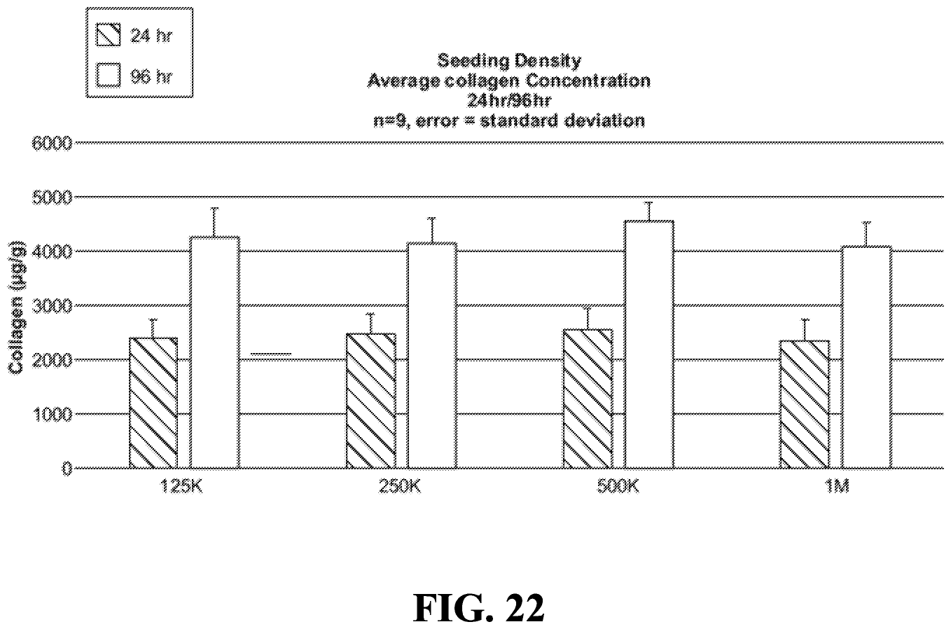

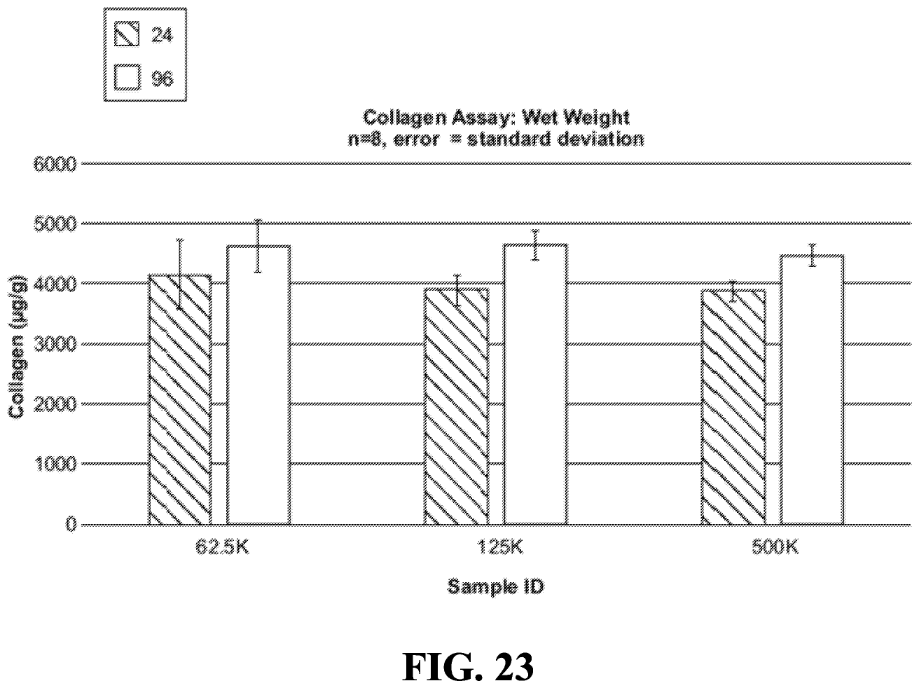

The effect of cell seeding density on tissue outcome was assessed in order to optimize the seeding concentration of bovine dermal fibroblasts. The optimization was completed on small scale PET scaffolds. Seeding densities of 125 k/cm², 250 k/cm², 500 k/cm², and 1M/cm² (as low as 30 k/cm² and as high as 1M/cm²) were examined after a 6.5 week incubation time (as short as 4 weeks or as long as 8 weeks) upon which samples were saved for collagen analysis and scanning electron microscopy (SEM) imaging. Collagen concentration was evaluated after a biopsy with an 8 mm biopsy punch followed by digestion for 24 or 96 hours. After neither time period was there a statistically significant difference in collagen concentration between any of the seeding densities. The same effects were examined upon scaling up to a seeding area of 220 cm² with seeding densities of 500 k/cm², 125 k/cm², and 62.5 k/cm². These seeded scaffolds were incubated for 8 weeks and processed similarly to assess collagen production. Following digestion for 24 or 96 hours, there was no statistically significant difference between the amount of collagen produced between the area seeded with a seeding density of 65 k/cm² and 125 k/cm² (FIG. 22 and FIG. 23) but there was a difference between the area seeded with 125 k/cm² and 500 k/cm² wherein collagen production increased with increased seeding density. FIG. 24 shows a substantially higher soluble collagen reading where a 30 k/cm² seeding density was used.

### Example 7. Tissue and Leather Growth Process

The cell source (immortalized/isolated cells) is thawed and grown out to reach the desired number of cells. The scaffolding material is prepared on a frame and sterilized along with the bioreactor containers. The cells are seeded (500 k cells/cm² fabric) onto the scaffolding material inside the bioreactor and tissue growth media is added before being allowed to incubate which is changed in a regular basis. To transform the grown tissue into leather, the engineered animal hides are removed from the growth medium and frames and optionally cleaned. Optionally, the hides can undergo salting, desalting, liming, or bating. The hides then undergo tanning to make leather which can optionally be followed by processing the hides (i.e., adding fat liquots, adding dyes, adding a re-tanning agent).

### Example 8. Fiber Diameter and Sterilization Parameter Effect on Fiber Strength

Fiber diameter can have a significant impact in the tactile output of the resulting leather, specifically in terms of its roughness. Furthermore, the roughness of the material and the fiber diameter can have an impact on the cell behavior. A series of PLA fiber diameters have been examined ranging from 1-40 dtex. Increasing the thickness of the fibers to 6.7 dtex from the control (100 gsm) as seen in Table 2 resulted in lower areal density, collagen, and DNA content.

TABLE 2

| | Control | 6.7 |
|---|---|---|
| | (100 gsm) | dtex |
| Assay | PLA | PLA |
| Glutamine consumption | 73.6 | 69.3 |
| [μM/cm²]* | | |
| Thickness [mm] | 1.44 | 1.36 |
| Areal Density [g/cm²] | 0.159 | 0.157 |
| Mean Pixel Value | 155 | 151 |
| [Pixel Value]* | | |
| Collagen [mg/biopsy] | 2.19 | 1.89 |
| DNA [μg/biopsy] | 27.2 | 31.2 |
| Dry Weight [mg/biopsy] | 6.16 | 10.5** |
| Histological Ingrowth | No | No |
| [yes/no] | | |
| Post Tan Grade | A | D |
| [A, B, C, D] | | |

### Example 9. Histological Images of Cross-Sections of Cell and Scaffold Compositions

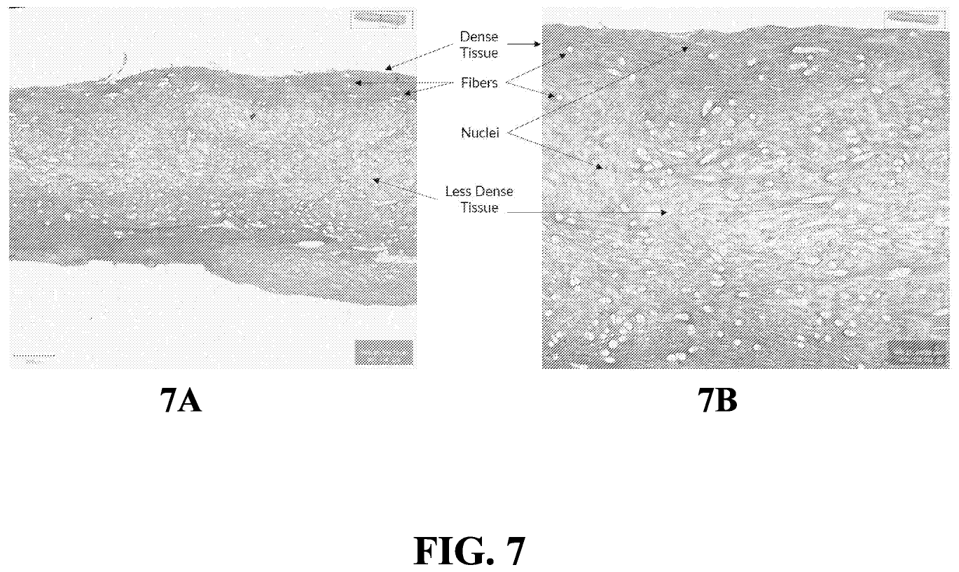

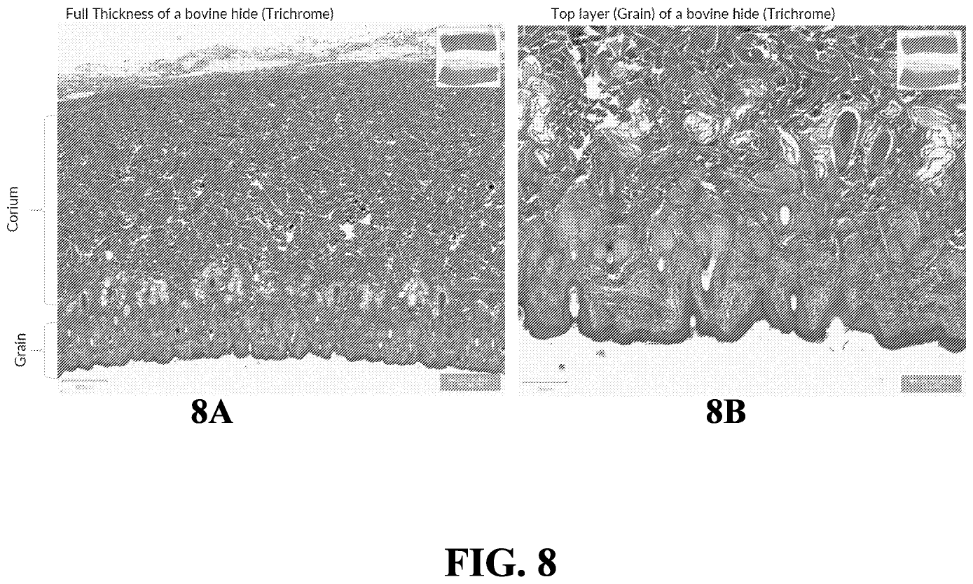

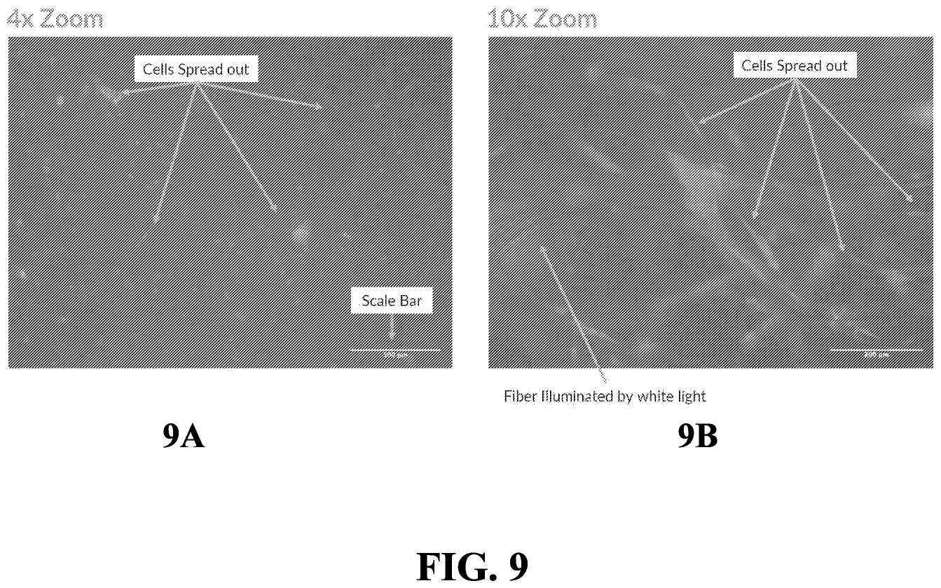

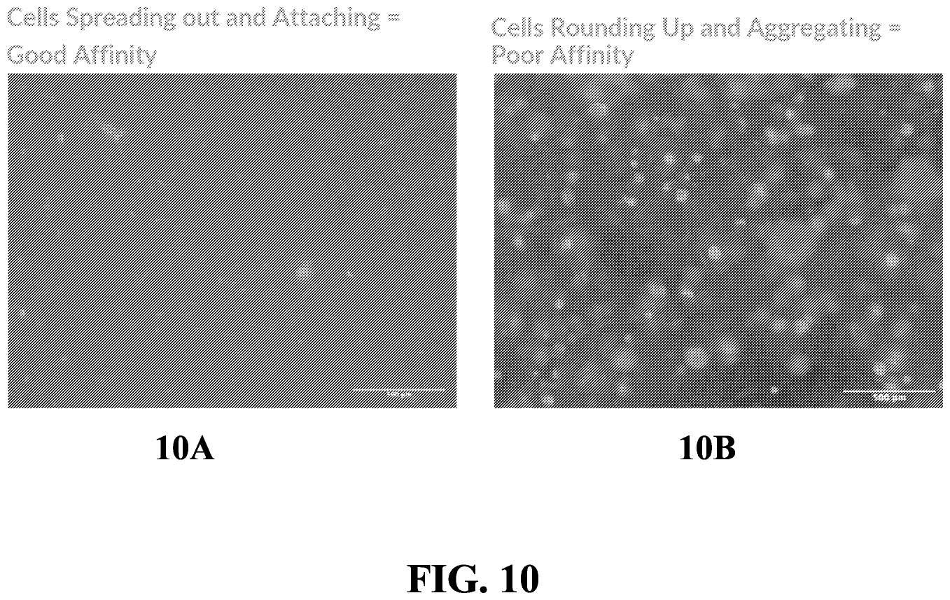

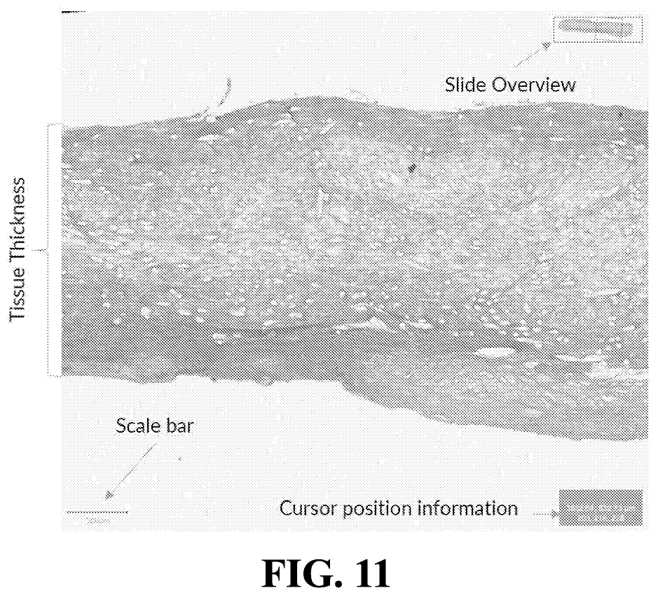

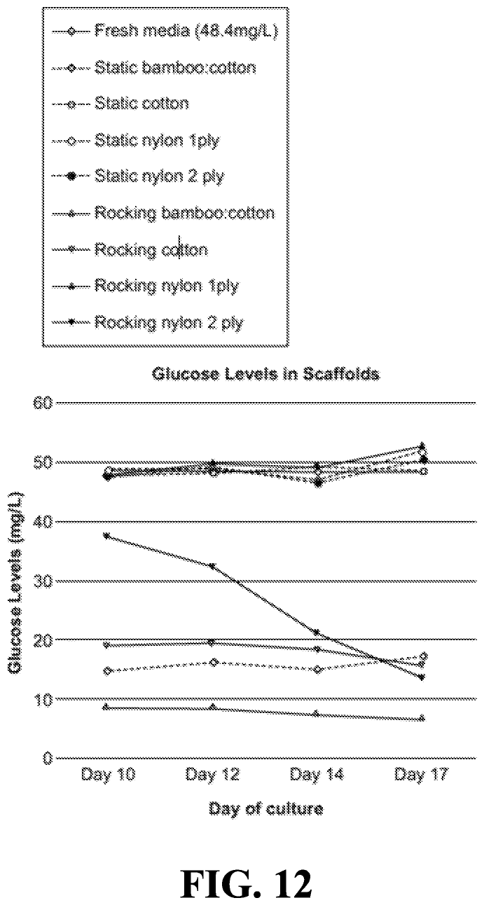

FIG. 7A and FIG. 7B show histological images of 5 μm cross-sections of tissues stained with trichrome stain on a scaffold with labels highlighting dense tissue, fibers, nuclei, and less dense tissue. FIG. 8A shows a histological image of a 5 μm cross-section of native bovine hide stained with a trichrome stain with labels indicating the location of the grain and the corium. FIG. 8B shows a histological image of a 5 μm cross-section of the top layer (grain) of a bovine hide stained with a trichrome stain. FIG. 9A shows a fluorescence microscopy image of fibroblast cells at 4× zoom after the addition of Calcein AM. The live cells fluoresce green and arrows highlight the spreading of cells indicating affinity for the scaffold. FIG. 9B shows a fluorescence microscopy image of fibroblast cells at 10× zoom after the addition of Calcein AM. The live cells fluoresce green and arrows highlight the spreading of cells indicating affinity for the scaffold. A second arrow highlights the fiber illuminated by white light. FIG. 10A shows a fluorescence microscopy image of fibroblast cells at 10× zoom after the addition of Calcein AM, causing the live cells to fluorescence green. The cells spreading and attaching highlights good affinity for the scaffold. FIG. 10B shows a fluorescence microscopy image of fibroblast cells at 10× zoom after the addition of Calcein AM, causing the live cells to fluoresce green. The cells rounding up and aggregating is indicative of poor affinity for the scaffold. FIG. 11 shows a histological image of a 5 μm cross-section of tissue stained with a trichrome stain with labels indicating the slide overview, scale bar, cursor position, and tissue thickness.

### Example 10. Basal Fiber Material Effects on Tissue Outcome

Cell attachment and tissue growth have been attempted on a variety of different materials. In this example, a variety of fiber types were tested first for cell attachment and then for tissue growth. A variety of sustainable biobased materials as well as less sustainable but more commercially available materials were tested. All materials tested here were needle punch nonwoven materials. A PET material was used as a control, as well as some nylon material. 2 different PLA materials, one known simply as PLA, and the other as 50-50 were tested. The 50-50 material contained 2 types of PLA fibers, each comprising 50% of the mass. Half were the normal high melting temperature PLA used in the first sample type. The other half of the fibers were a special core-sheath fiber, in which the core was a high melting temperature polymer, and the outer layer or sheath was a lower melting temperature polymer. This special fiber construction allows for needle punch nonwoven fabric that is more easily fabricated into the desired shape, as any heat treatment will easily melt or soften the sheath material while maintaining the integrity of the core material. Two viscose materials were tested, which had different cross sections, circular and trilobal, which were denoted as regular and trilobal viscose respectively. Viscose is a cellulose fiber which is created by dissolving wood or other cellulosic fibers in a caustic sulfide solution, then reconstituting the dissolved pulp as fibers. Lastly a lyocell material was tested. Lyocell, unlike viscose, is dissolved by a solvent process which allows for a drastic reduction in toxic waste materials compared to viscose.

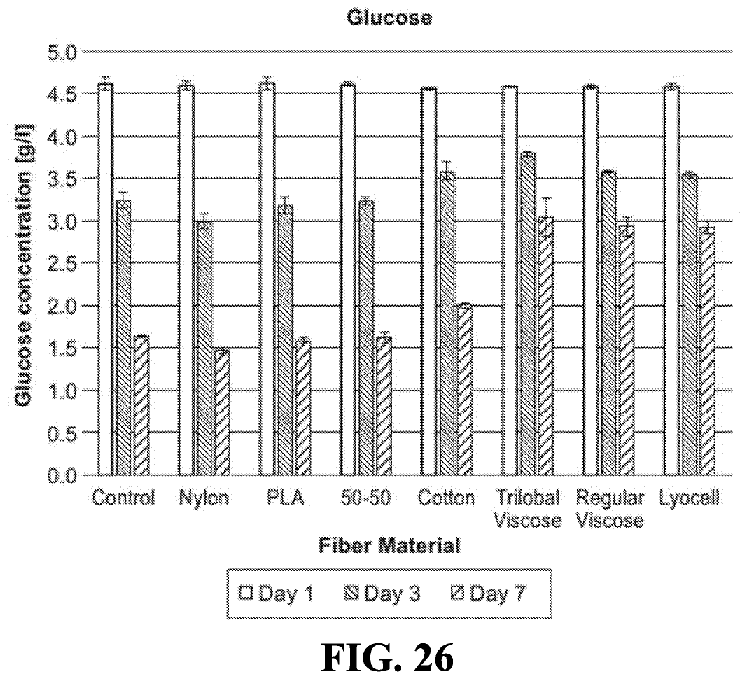

Initial attachment tests showed high glucose consumption by 7 days of culturing in the case of PET, Nylon, PET, and 50-50 scaffolds, as seen in FIG. 26. Glucose consumption, similar to MTT, can be used as an indirect measurement of cell affinity for a substrate, as significant glucose consumption & cell activity is only achieved with sufficient affinity of the cell for its substrate. The cellulose-based materials showed less glucose consumption than the previously mentioned materials. Of the cellulose-based materials, cotton showed the highest consumption, but this lagged behind the other materials previously mentioned, as seen FIG. 26. While in these culture conditions, it was found that cells did not find a high affinity for the surface of cellulose-based materials. However, upon switching culture medium, cells were found to attach to the Lyocell based materials.

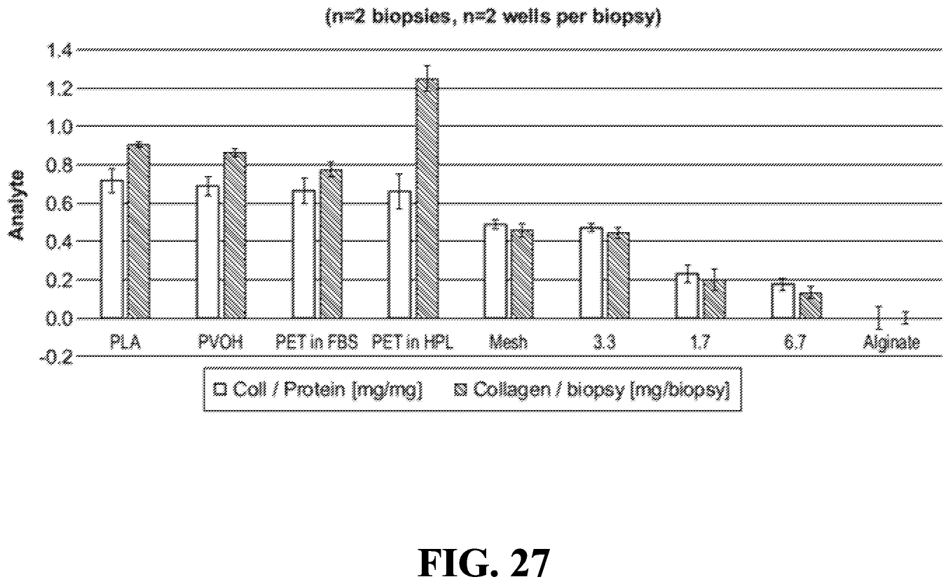

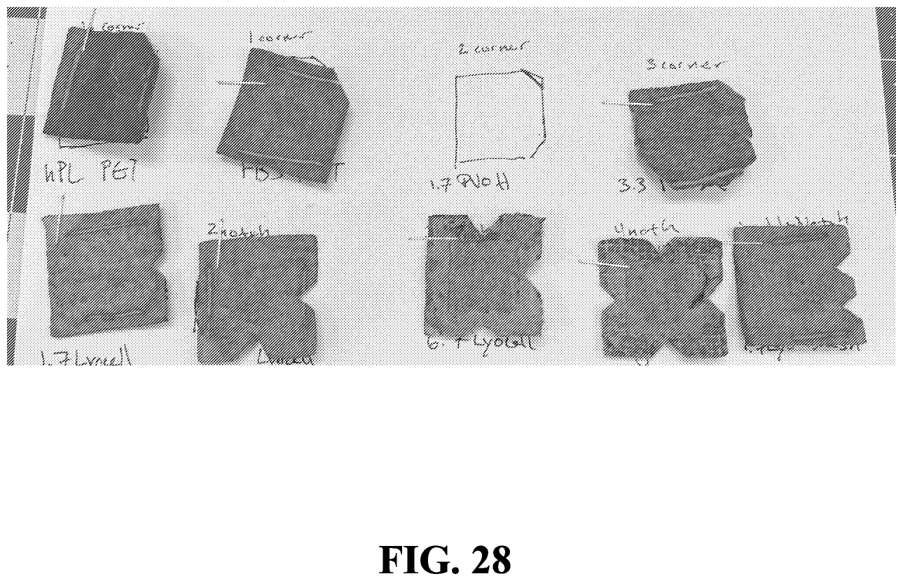

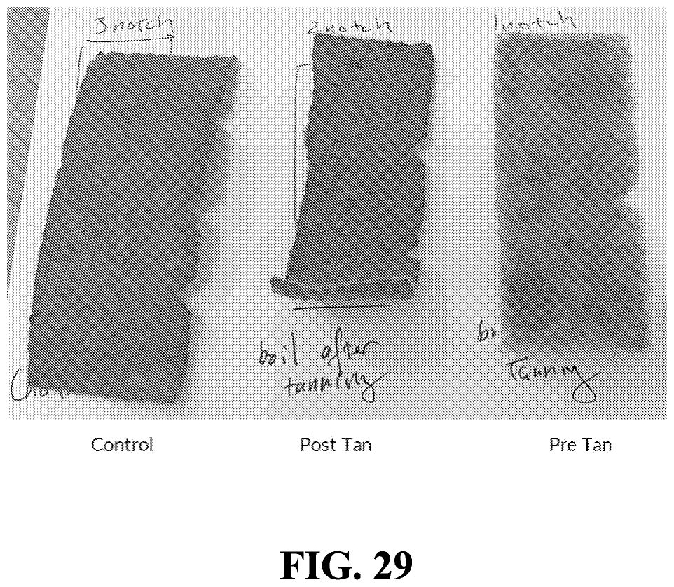

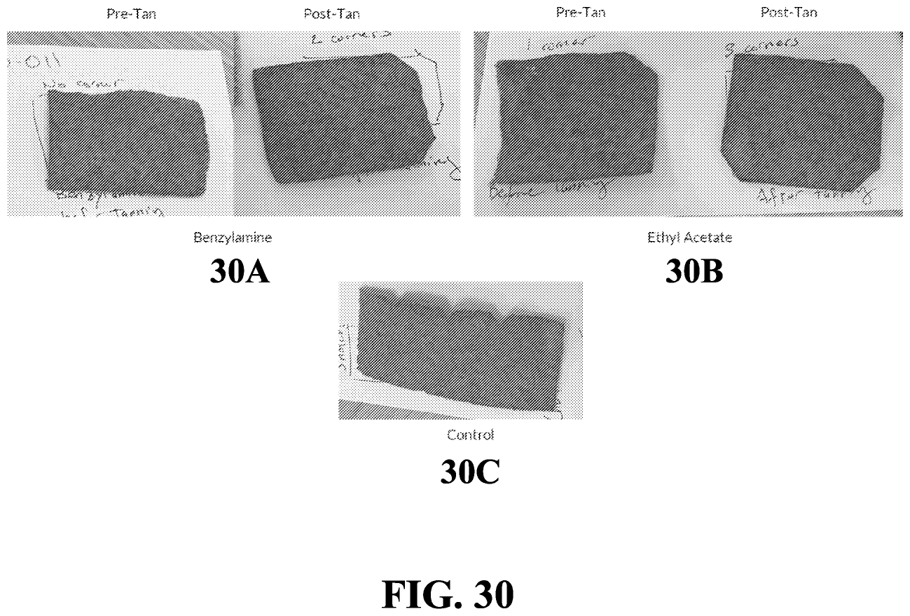

In a follow-on tissue culture experiment additional materials were tested. A scaffold comprised of polyvinyl alcohol, also known as PVOH or PVA, as well as 3 different lyocell scaffolds were tested. These 3 new lyocell scaffolds used different weight and thus diameter fibers, including 1.7, 3.3, and 6.7 dtex fibers. In addition, a scaffold comprised of alginate was tested. Another scaffold comprised of Vicryl mesh, which is comprised of PLGA fibers knitted into a mesh. The total collagen content and collagen content normalized by total protein content were evaluated for each of the tissues grown. The highest collagen per biopsy was realized in the PET hPL condition, showing the impact of both media conditions in addition to scaffold conditions on the tissue deposited as can be seen in FIG. 27. Tissues grown on PLA and PVOH had the next most amounts of collagen, followed by PET in FBS, then the Vicryl Mesh. The different Lyocell materials with their different fiber weights scored quite differently from each other. The 3.3 dtex scaffold seemed to produce the most collagen of the three, which 1.7 dtex and 6.7 dtex seemed to have very little tissue deposited. Least of all, the scaffold with Alginate produced almost no collagen at all. The small-scale square tissues were then tanned, marked so that they could be distinguished from each other, as can be seen in FIG. 28. While hPL PET did produce the most collagen and seems to have been dyed the most deeply.

### Example 11. Scaffold Form Factors Effect on Tissue Outcomes

A number of biomaterial scaffold form factors are tested herein, and their effects upon tissue formation are evaluated. The control used for this experiment is a simple needlepunch nonwoven material which has been pressed on one side by a heated plated to a given thickness, such that one face is smooth; this condition is referred to as “Pressed Control.” A 3M Thinsulate material Type G is tested here, which is comprised of 3 main layers, 2 spun-bond, and one air-laid nonwoven, which are sandwiched, such that it is a layer of spun-bond, then air-laid, then spun-bond again. The material is a polyester and is likely coated with a variety of materials to allow for maximal insulation properties. The material is also held together at an interval by lines of stitching, and for the sake of this experiment sutures were applied to create a more regular thickness of the material throughout. This condition will be referred to as “Thinsulate.” Next is a towel like material made of PET material which will be referred to as “Secant Towel.” This material can be seen in closer detail in FIG. 41A-B. Here the pattern of looping fibers stemming from a flat knitted face can be seen, which is a form of a terry cloth. Next is a thicker material which is comprised of two fabrics which are held apart by fibers running between the two spacing the two outer fabrics by a set distance. These types of fibers are colloquially known as spacer fabrics and will be referred to here as “Secant Spacer.” Next is “Fibertex” which is another needlepunch nonwoven fabric. However, this fabric unlike “Pressed Control” was not pressed; this fabric is comprised of only a single fiber population of lyocell fibers, unlike the control which is composed of a suite of PET fibers. Next is “Double stack of Pressed” in which two of the “Pressed Control” fabrics are laminated one on top of the other with the rough surfaces facing inwards toward each other with the smooth sides facing out. The two fabrics are laminated together by sutures. Next is “Smooth Facing Laminate” where a Vicryl mesh sheet is laminated on top of a “Pressed Control” fabric by way of sutures, such that the Vicryl mesh is adjacent to the smooth face of the “Pressed Control” fabric. Next is “Rough Facing Laminate,” which is identical to “Smooth Facing Laminate,” except for the arrangement of the mesh and the “Pressed Control” fabric, as the rough side of the “Pressed Control” fabric is now adjacent to the mesh. Next is “Autoclaved Laminate” which is a duplicate of rough facing Laminate except the scaffold has been laminated together and then autoclaved, where the others were sterilized prior to assembly. Lastly is “Absorbable only” in which two Vicryl Meshes are laminated together, such that the entirety of the scaffold is comprised of knitted absorbable materials.

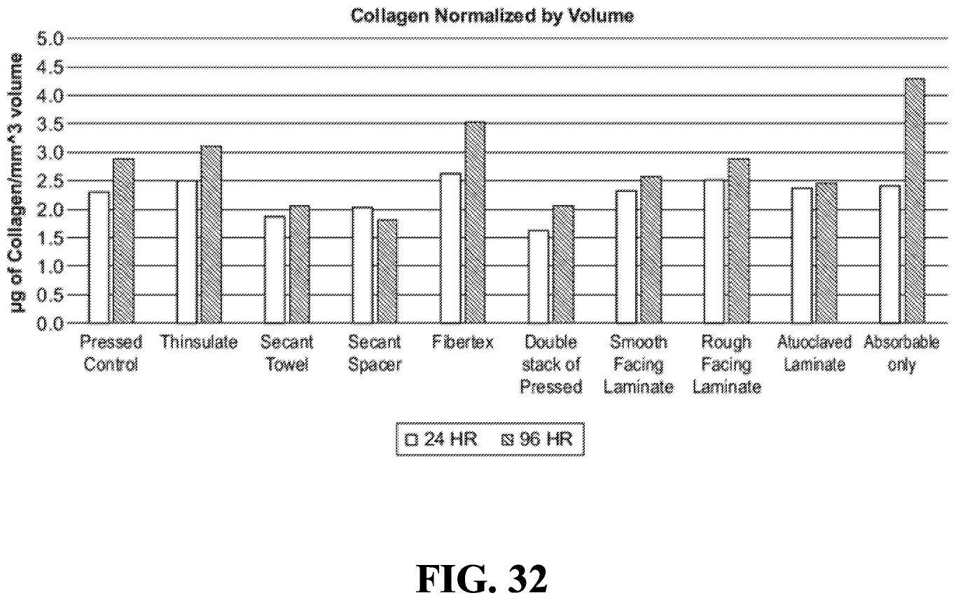

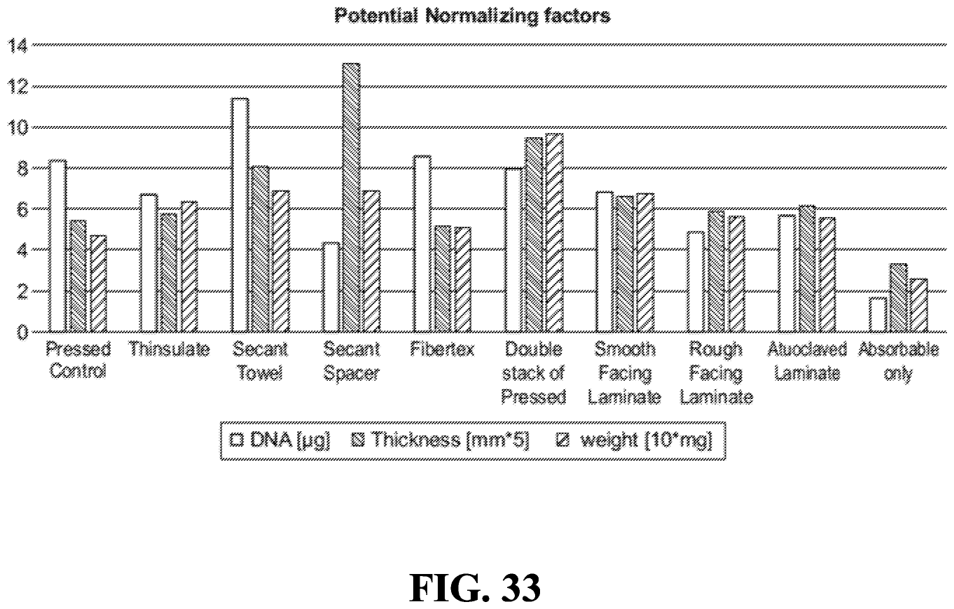

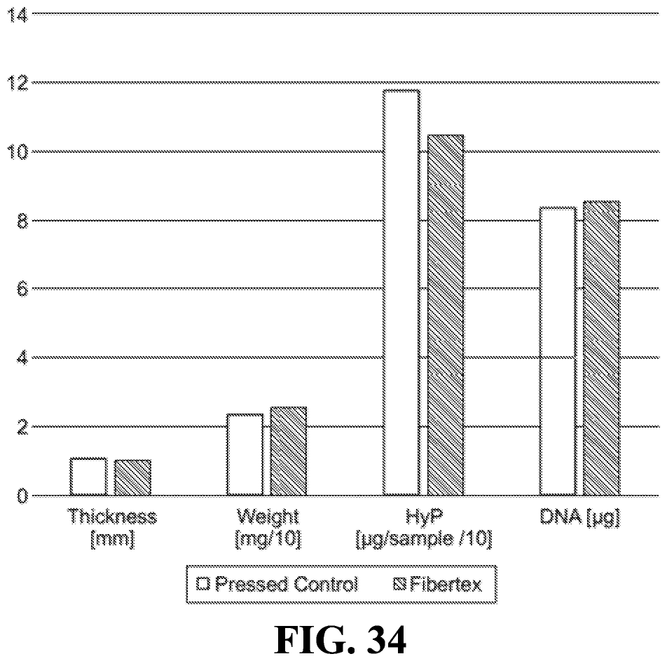

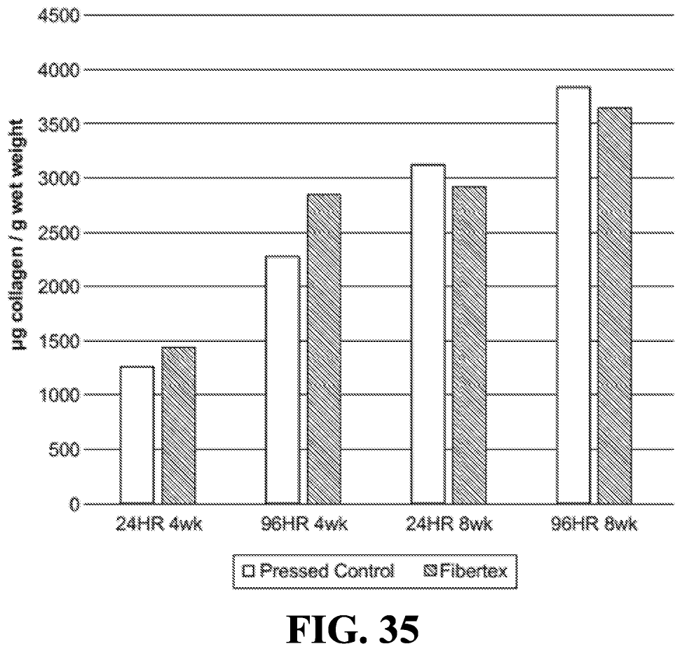

After a full culture period on cardholder scale materials, the hides were analyzed for their biochemical content and physical properties. When normalized by volume of the biopsy taken, the “Absorbable Only” condition yielded the highest concentration of collagen at the 96-hour digestion time point as seen in FIG. 32, but this result can be discounted by the fact that the thickness, and thus volume of the biopsy, would have been smaller than any other condition, as seen in FIG. 33. Thinsulate, Fibertex, and Rough Facing Laminate would have then next highest collagen densities of the form factors tested, as seen in FIG. 32. When looking at other physical and biochemical features, it can be seen that Secant Towel had the highest DNA content, while Secant Spacer had the highest thickness. Double stack of pressed by contrast had high DNA, Thickness, and weight content compared to other conditions. In FIG. 34 Fibertex can be seen directly compared against the Pressed Control condition, in which the Thickness, weight, Hydroxyproline, and DNA content can all be seen to be very similar. This makes sense as the form factors of the scaffold sample are the most similar. In FIG. 35 the collagen values after 4 weeks and 8 weeks of tissue growth are compared between Pressed Control and Fibertex. Unlike by the Hydroxyproline measurement taken at 8 weeks, the soluble collagen is found to be slightly lower in Fibertex at 8 weeks, but higher at 4 weeks. Due to the slightly different internal structures of Fibertex compared to Pressed Control, the differences in growth kinetics can be expected.

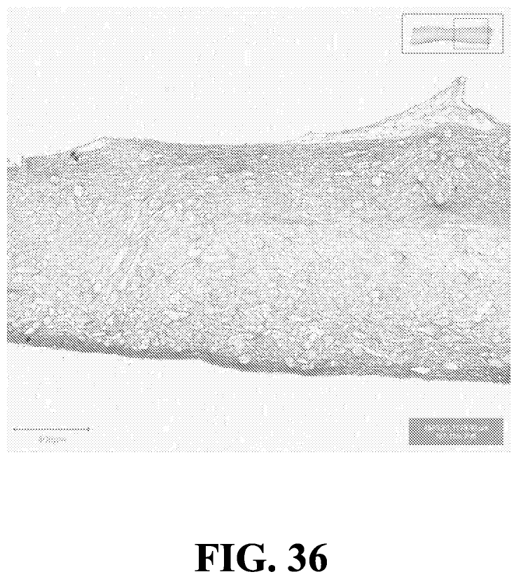

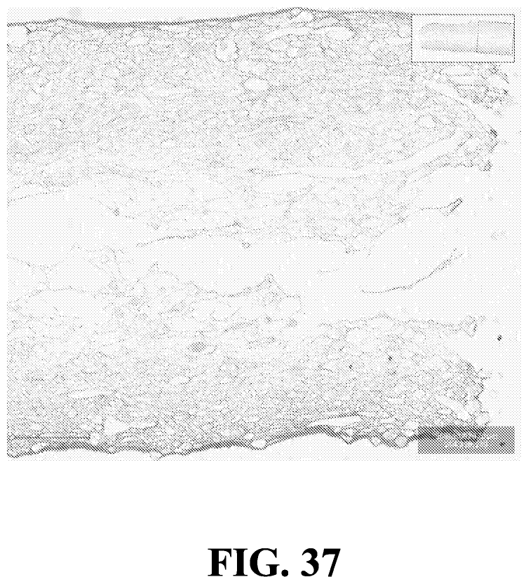

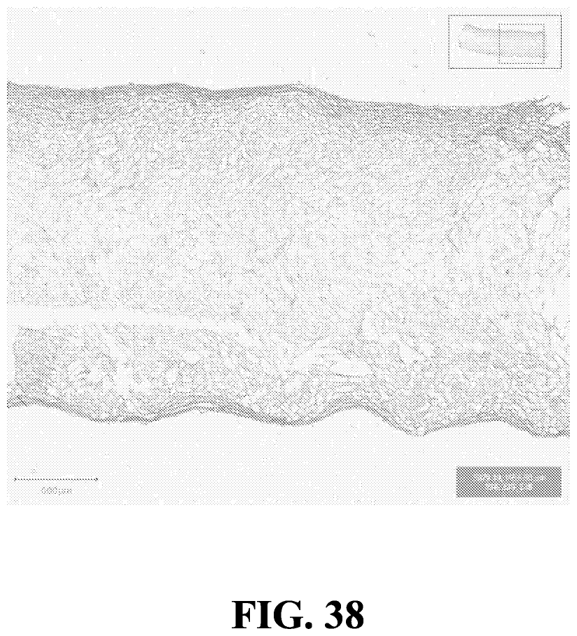

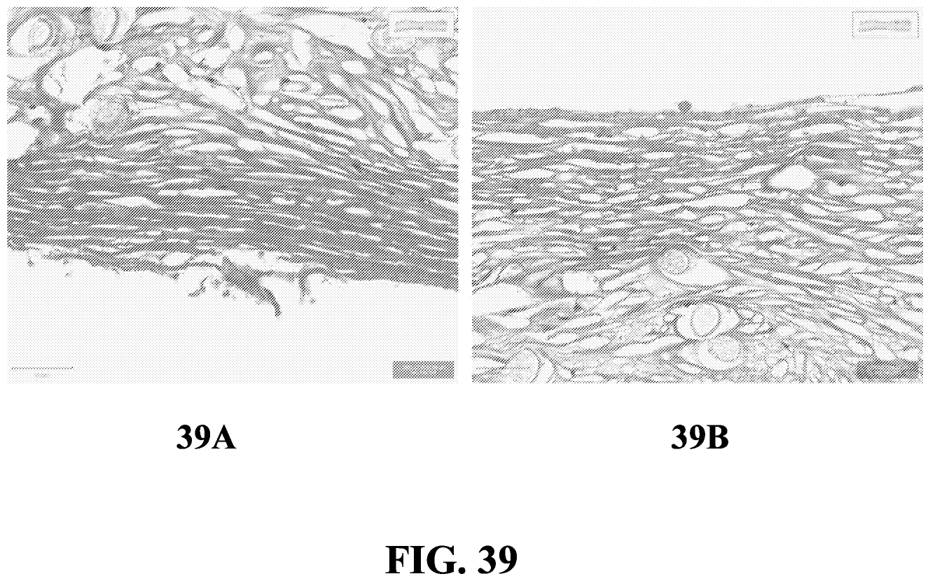

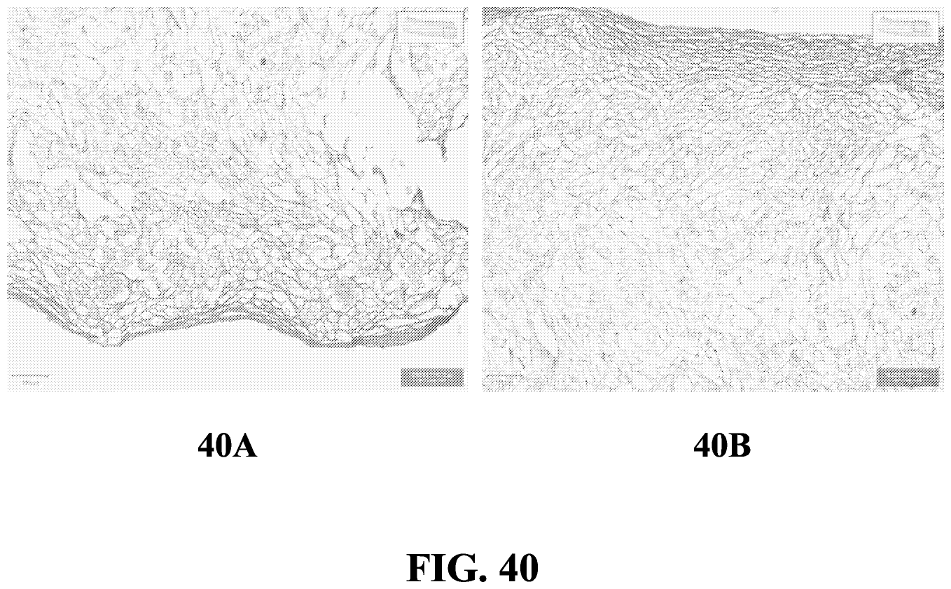

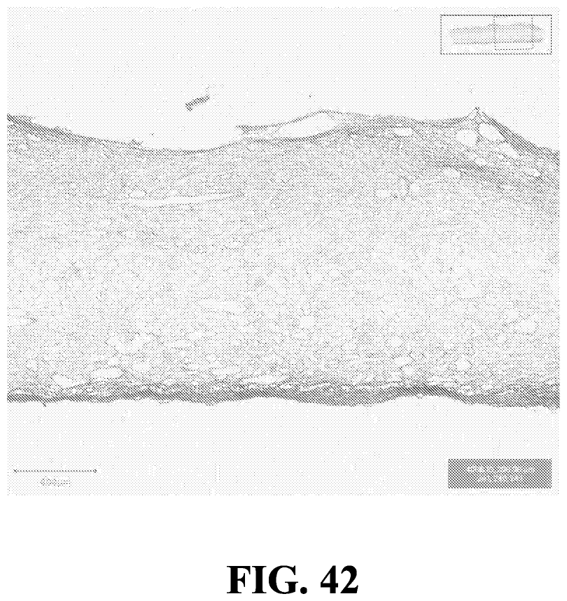

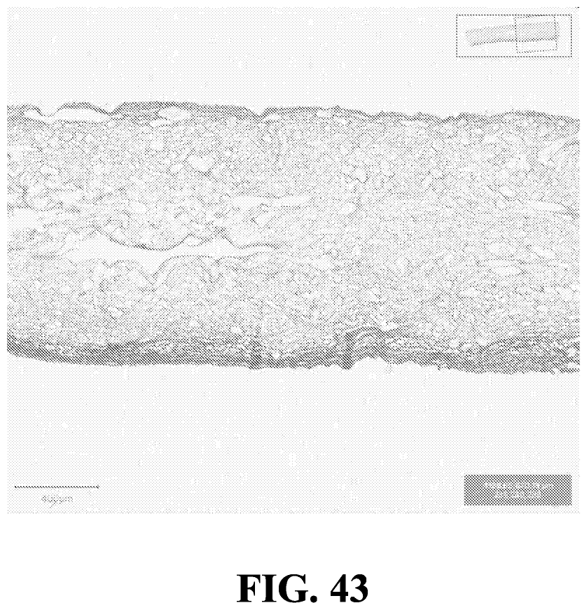

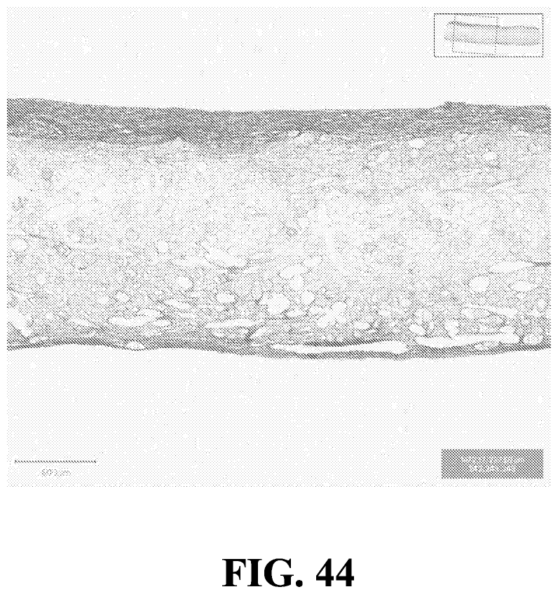

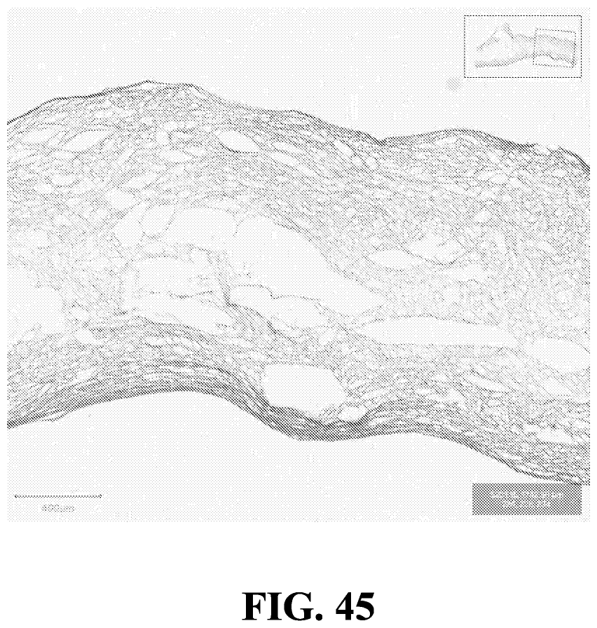

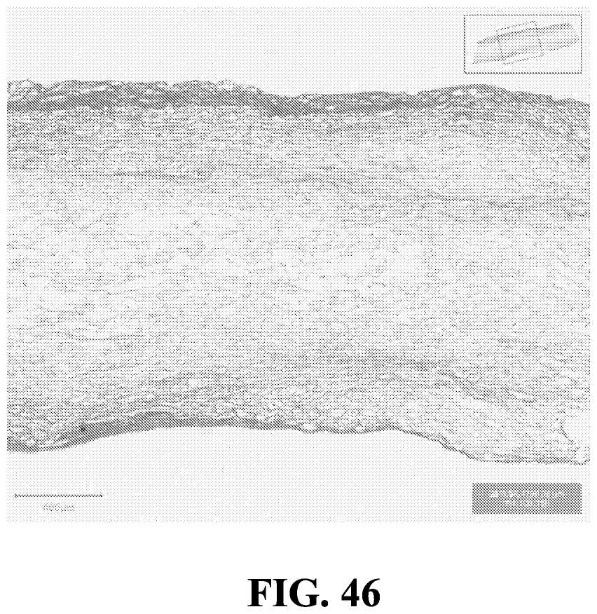

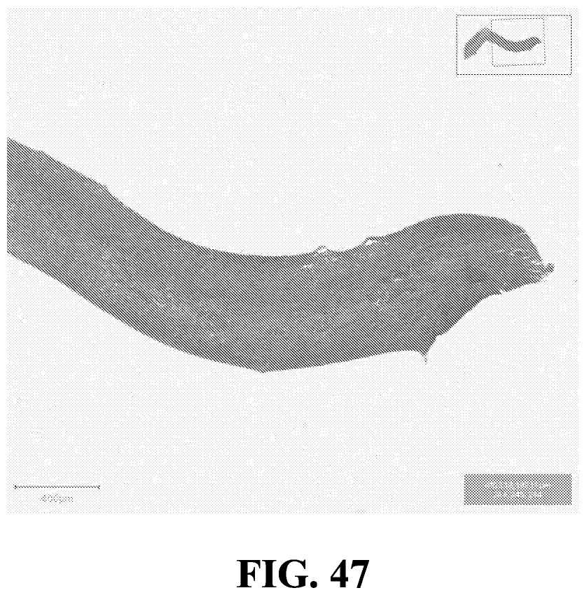

In addition to changes in biochemistry, these different form factors were shown to impact the way the tissue formed in three-dimensions. By use of histology, it can be observed how the tissue formed differently with the different scaffolds. In the Pressed Control, a relatively even distribution of tissue throughout the thickness of the scaffold with a slightly denser deposition of tissue on one surface can be seen in FIG. 36. The Double Stack of Pressed scaffold by contrast had a very distinct gradient of tissue deposition, with a high density on the edges and very little in the center, with some completely empty regions, as seen in FIG. 37. Double Stack of Pressed scaffold was much more thick than Pressed Control, which insinuates the impact of the thickness of the material. The Secant Towel material, similar to Pressed Control resulted in a tissue with a relatively continuous deposition of tissue, as seen in FIG. 38. Unlike Pressed Control, Secant Towel seems to have a distinct surface pattern on one side where there is a wavy surface. Near the crests there is a high density of fibers, and in the troughs, there are few fibers, if any, present. The local collagen distribution of Pressed Control and Secant Towel can be compared. In Pressed Control the collagen deposited on the surfaces is very planar in nature as seen in FIG. 39A-B, whereas in Secant Towel, the collagen deposition is much more random, and the arrangement appears more circular and isotropic by comparison as seen in FIG. 40A-B. Smooth Facing and Rough Facing laminates can be distinguished in their tissue deposition pattern by the nature of the outer two surfaces created in the tissue deposition. In Smooth Facing Laminate, where the rough face of the Pressed Control is facing out, the histological cross section shows one smooth face and one rough face, as seen in FIG. 42. In the Rough Facing Laminate by comparison, where the rough face of the Pressed Control was facing in, two smooth outer surfaces in the histological cross section, can be seen in FIG. 43. In Autoclaved Laminate, two smooth faces, similar to Rough Facing Laminate in FIG. 44, however, a denser layer of collagen on one face is seen, likely due to the difference in Vicryl character after autoclaving which may have caused an amount of hydrolytic degradation prior to seeding. In Fibertex a bit of a different tissue deposition compared to Pressed Control is seen. A bit of discontinuous tissue formation is seen where there are voids torn open in the histological cross section, however outside of these tears there is relatively continuous tissue deposition, as seen in FIG. 45. In the sandwich structure from Thinsulate a slightly thicker tissue cross section is seen, but relatively continuous tissue deposition throughout the thickness. Despite the tighter network of fibers on the top and bottom surfaces from the spun-bond layers, good tissue penetration through the surface is seen and there does not appear to be an impermeable layer in FIG. 46. Lastly in the absorbable tissue very dense tissue formation is seen that is not seen in any of the other tissues, in FIG. 47. This material unlike the others was very thin to start off with, had very high cell affinity, and dissolved away by the end of culture. All of these factors likely resulted in the high tissue density observed both through the histology and biochemistry data. To assist in interpretation, please see FIG. 11 for better understanding of the anatomy of a histology image.

Through this experiment a variety of form factors were tested, including needle punch nonwovens, 3-layer sandwiches, mesh and nonwoven laminates, terry cloth, spacer fabrics, and pure knit meshes. Other form factors have been tested such as woven meshes as seen in Example 1. The diverse effects that these form factors can have on tissue formation can be seen. The various effects like an empty tissue center in the case of the Double Stack and the incredibly dense tissue in the case of the Absorbable Only, as well as the continuous tissue despite lack of present fibers in the towel like fabric, may all be utilized to achieve the desired tissue characteristics. The data indicate that form factor may have effects on tissue outcomes.

### Example 12. Scaffold Attachment Factor Effects on Leather Outcome

Polylactic acid (PLA) and modifications of the PLA scaffold surfaces are tested to evaluate efficacy of covalent and non-covalent attachment factors on the surfaces. A polylactic acid (PLA) scaffold is coated with glycidyl trimethylammonium chloride (GTAMC), which allows for introduction of amines on the surface of the polymer (PLA-NH₂), which can undergo non-specific adsorption to collagen. A polylactic acid scaffold is coated with pea protein and subjected to hydrolysis in acidic conditions to expose hydroxyl groups, carboxylic acids, and ketones (PLA-hyd), which can subsequently undergo non-specific adsorption of collagen. A polylactic acid scaffold is coated with pea protein and also oxidized by use of sodium periodate (PLA-ox), allowing for increased non-specific adsorption of collagen. To each of the three scaffolds and the PLA control, fibroblasts are seeded and grown to produce an extracellular matrix, which includes collagen. The scaffold is optionally removed by way of dissolution in benzylamine, ethyl acetate, or acetone. The resulting material is examined by, for example, histology and microscopy to determine density and distribution of the resulting collagen in view of the aforementioned scaffold modifications. The resulting materials may undergo tanning to result in a cruelty-free leather and hide thickness and strength can be compared.

In another example, a polylactic acid (PLA) scaffold is coated with a chemically modifiable polymeric material. In one example, the polymeric material coated PLA is further modified by addition of a carbodiimide linker, EDC (PLA-EDC). The PLA-EDC is seeded with fibroblasts and the fibroblasts are grown, resulting in the formation of an extracellular matrix comprising collagen. The collagen is modified with N-hydroxysuccinimide. The collagen and carbodiimide linker of the PLA-EDC are chemically cross-linked to result in a covalently bound scaffold-polymer material. In another example, the polymeric material coated PLA is further modified by addition of an azide (—N₃) (PLA-N₃). The PLA-N₃ is seeded with fibroblasts, and the fibroblasts are grown resulting in an extracellular matrix comprising collagen. The collagen is then modified with an alkyne which is cross-linked to the azide in a click-chemistry reaction to result in a covalently cross-linked scaffold-collagen material. The PLA-N₃-collagen and PLA-EDC-collagen materials are subsequently, optionally, decellularized before examination and comparison to the PLA control via histology and microscopy to examine collagen density and distribution. The resulting materials also may optionally be tanned, resulting in cruelty-free leather, and hide thickness and strength can be compared to the PLA control.

### Example 13. Hide Strength and Thickness

Average double tear strength and thickness of hides prepared with varying scaffolds were measured and compared to that of typical cow hide crust. The average double tear strength was measured using ISO 3377-2 method. The results of the measurements can be found in Table 3.

TABLE 3

| | Average | |
|---|---|---|
| | Double Tear | Thickness |
| | Strength (N) | (mm) |
| Tanned hide w/ PLA | 3 | 0.4-0.6 |
| scaffold | | |
| Tanned hide w/ rayon | 19 | 0.4-0.6 |
| scaffold | | |
| Tanned hide w/ PET | 31 | 0.4-0.6 |
| scaffold | | |
| Typical cow hide crust | 20-25 | 1.0-1.2 |

The resulting double tear strength and thickness may indicate that the compositions provided herein comprising a scaffold in contact with the extra-cellular matrix, and the methods provided herein, may result in scaffolds with strength that can be adjusted merely by adjusting the scaffold material. Additionally, the data suggest, when comparing to cow hide crust, that the compositions provided herein may comprise similar strengths as natural hides but in thinner materials.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising a scaffold in contact with an extracellular matrix comprising a collagen, wherein the scaffold comprises a non-woven, needle-punched material.

2. A composition comprising a scaffold in contact with an extracellular matrix comprising a collagen, wherein the scaffold comprises a three-dimensional woven material.

3. A composition comprising a scaffold in contact with an extracellular matrix comprising a collagen, wherein the scaffold comprises a polycaprolactone (PCL), a polylactic acid (PLA), a polylactic and polyglycolic acid (PLGA), a polyethylene terephthalate (PET), a nylon, a polyethylene (PE), a polyethylene furanoate (PEF), a polypropylene (PP), a polyvinyl alcohol (PVA), a cotton, a bast fiber, a viscose, a modal, a lyocell, a plant based protein fiber, a biobased material, a viscose, a cellulose, an alginate fiber, a thermoplastic starch, or any combination thereof.

4. A composition comprising an at least partially coated scaffold in contact with an extracellular matrix comprising a collagen, wherein the scaffold is at least partially coated with a coating comprising a matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, or any combination thereof.

5. A composition comprising a dissolvable scaffold in contact with an extracellular matrix comprising a collagen, wherein the scaffold is in contact with a solvent that can dissolve the scaffold.

6. The composition of any one of claims 1, 2, 4, or 5, wherein the scaffold comprises a polycaprolactone (PCL), a polylactic acid (PLA), a polylactic and polyglycolic acid (PLGA), a polyethylene terephthalate (PET), a nylon, a polyethylene (PE), a polyethylene furanoate (PEF), a polypropylene (PP), a polyvinyl alcohol (PVA), polyhydroxyalkanoate (PHA), polyhydroxybutyrate (PHB), oxidized alginate, a cotton, a bast fiber, a viscose, a modal, a lyocell, a plant based protein fiber, a biobased material, a viscose, a cellulose, an alginate fiber, a thermoplastic starch, or any combination thereof.

7. The composition of any one of claims 2-6, wherein the scaffold comprises a non-woven, needle-punched material.

8. The composition of claim 1 or claim 7, wherein the non-woven, needle punched material comprises a first plurality of pores.

9. The composition of claim 8, wherein the scaffold comprises a non-woven configuration that creates the first plurality of pores.

10. The composition of claim 9, wherein the first plurality of pores comprise an average pore size of about 30-70 μm.

11. The composition of claim 9, wherein the first plurality of pores comprise an average pore size of about 80-120 μm.

12. The composition of claim 9, wherein the first plurality of pores comprise an average pore size of about 50 μm or about 100 μm.

13. The composition of any one of claims 8-12, wherein the scaffold comprises a second plurality of pores produced by a needle punch.

14. The composition of any one of claims 1-3 or 5, wherein the scaffold is at least partially coated with a coating comprising a Matrigel, a vitronectin, a fibronectin, a protein extract from a soy, a protein extract from a pea, a protein extract from a corn, a peptide generated from synthesis, an RNA-binding glycine-rich (RBG) protein, a polylysine, a synthetic protein, an RGD peptide, a polylysine, a polyarginine, a polyornithine, a recombinant protein, an oligomer, a polymer, glycidyl trimethylammonium chloride (GTMAC), carbohydrate-binding module, a cellulose binding domain, a starch binding domain, or a combination thereof.

15. The composition of claim 4 or 14, wherein the scaffold comprises an amine hydroxyl group, a sulfhydryl group, a tyrosyl group, or a carboxylic acid group.

16. The composition of any one of claims 4, 14 or 15 wherein the scaffold is at least partially coated with the carbohydrate-binding module.

17. The composition of any one of claims 14-16, wherein the carbohydrate-binding module is a cellulose-binding domain.

18. The composition of claim 17, wherein the scaffold comprises cellulose.

19. The composition of claim 16, wherein the carbohydrate-binding module is a starch-binding module.

20. The composition of claim 19, wherein the scaffold comprises starch.

21. The composition of any one of claims 16-20, wherein the carbohydrate-binding module is associated with an enzyme.

22. The composition of claim 21, wherein the enzyme does not hydrolyze the scaffold.

23. The composition of any one of claim 4 or claim 14-22, wherein the coating comprises a modification.

24. The composition of claim 23, wherein the modification comprises a reductive modification, an additive modification, or a combination thereof.

25. The composition of claim 23, wherein the modification of the scaffold increases a strength of a non-specific adsorption of the collagen to the scaffold.

26. The composition of claim 23, wherein the modification comprises hydrolysis.

27. The composition of claim 26, wherein the modification exposes chemically active groups comprising hydroxyls, carboxylic acids, ketones, or a combination thereof.

28. The composition of claim 23, wherein the modification comprises oxidation.

29. The composition of claim 28, wherein the oxidation occurs with sodium periodate.

30. The composition of any one of claims 23-29, wherein the scaffold comprises an amine, carboxylic acid, sulfate, aldehyde, hydrazide, sulfhydryl, diazirine, aryl-azide, acrylate, or epoxide.

31. The composition of any one of claims 14-30, wherein the scaffold is at least partially coated with a coating comprising the GTMAC.

32. The composition of claim 31, wherein the scaffold comprises primary amines.

33. The composition of claim 31 or 32, wherein at least partially coating with a coating comprising GTAMC increases the surface charge of the scaffold.

34. The composition of any one of claims 1-33, wherein the collagen is associated with the scaffold.

35. The composition of any one of claims 1-34, wherein the collagen is associated with the scaffold through non-specific adsorption.

36. The composition of any one of claims 1-35, wherein the collagen is associated with the scaffold through Van der Waals interactions.

37. The composition of any one of claims 1-36, wherein the collagen is associated with the scaffold through hydrogen bonding.

38. The composition of any one of claims 1-37, wherein the collagen is associated with the scaffold through depletion interactions.

39. The composition of any one of claims 1-38, wherein the collagen is associated with the scaffold through electrostatic interactions.

40. The composition of any one of claims 1-34, wherein the collagen is associated with the scaffold through covalent interactions.

41. The composition of any one of claims 1-40, wherein the scaffold comprises a carbodiimide or an N-hydroxysuccinimide ester (NHS ester).

42. The composition of any one of claims 1-41, wherein the collagen comprises a carbodiimide or an N-hydroxysuccinimide ester (NHS ester).

43. The composition of claim 41 or 42, wherein the carbodiimide is N,N′-Dicyclohexylcarbodiimide (DCC) or 1-Ethyl-3-(3-dimethylaminopropyl)carbodiimide (EDC).

44. The composition of any one of claims 41-43, wherein the collagen is associated with the scaffold through EDC/NHS coupling.

45. The composition of any one of claims 1-34, wherein the scaffold comprises an azide or an alkyne.

46. The composition of any one of claims 1-34 or 45, wherein the collagen comprises an azide or alkyne.

47. The composition of any one of claims 45 or 46, wherein the collagen is associated with the scaffold through a click chemistry reaction.

48. The composition of any one of claims 1-34, wherein the scaffold comprises a Michael donor or a Michael acceptor.

49. The composition of any one of claims 1-34 or 48, wherein the collagen comprises a Michael donor or a Michael acceptor.

50. The composition of any one of claims 48 or 49, wherein the collagen is associated with the scaffold through coupling of the Michael donor and the Michael acceptor.

51. The composition of any one of claims 48-50, wherein the Michael donor comprises an enolate.

52. The composition of any one of claims 48-51, wherein the Michael acceptor comprises an α,β-unsaturated carbonyl.

53. The composition of any one of claims 1-34, wherein the scaffold comprises a thiol or maleimide.

54. The composition of any one of claims 1-34 or 53, wherein the collagen comprises a thiol or maleimide.

55. The composition of any one of claims 53 or 54, wherein the collagen is associated with the scaffold through coupling of the thiol and maleimide.

56. The composition of any one of claims 53-55, wherein the collagen is associated with the scaffold in the presence of (sulfosuccinimidyl 4-(N-maleimidomethyl)cyclohexane-1-carboxylate) (sulfo-SMCC), wherein the scaffold and collagen comprise a thiol.

57. The composition of any one of claims 1-34, wherein the collagen is associated with the scaffold through a Maillard reaction.

58. The composition of any one of claims 1-4, or 6-57, wherein the scaffold comprises a dissolvable scaffold, wherein the scaffold is in contact with a solvent that can dissolve the scaffold.

59. The composition of claim 5 or 58, wherein a temperature of the solvent is below the boiling point of the solvent.

60. The composition of claim 58 or 59, wherein the solvent is at a temperature of about 40° C. to about 50° C.

61. The composition of claim 58 or 59, wherein the scaffold comprises a thermoplastic polymer.

62. The composition of claim 61, wherein the thermoplastic polymer comprises a polyvinyl alcohol (PVA).

63. The composition of claim 61, wherein the thermoplastic polymer comprises a polyvinyl alcohol (PVA) or polyvinyl alcohol (PVOH).

64. The composition of claim 61, wherein the thermoplastic polymer comprises polylactic acid (PLA).

65. The composition of any one of claims 58-64, wherein the solvent comprises water.

66. The composition of any one of claims 58-64, wherein the solvent comprises an organic solvent.

67. The composition of claim 66, wherein the organic solvent comprises acetone, benzylamine, or ethyl acetate.

68. The composition of claim 67, wherein the solvent comprises a benzylamine.

69. The composition of claim 66, wherein the scaffold comprises a polylactic acid (PLA), and wherein the solvent comprises a benzyl group, an ethyl group, a haloalkane, or a combination thereof.

70. The composition of any one of claims 58-68, wherein the scaffold is dissolved over time through hydrolytic degradation.

71. The composition of any one of claims 58-70, wherein the scaffold is degraded over time through hydrolytic degradation in a neutral aqueous solution.

72. The composition of any one of claims 58-71, wherein the solvent comprises a dissolution agent.

73. The composition of claim 72, wherein the dissolution agent comprises ethylenediaminetetraacetic acid (EDTA).

74. The composition of claim 73, wherein the scaffold comprises an alginate.

75. The composition of claim 72, wherein the dissolution agent comprises a strong acid.

76. The composition of claim 75, wherein the strong acid comprises hydrochloric acid, nitric acid, hydroiodic acid, perchloric acid, chloric acid, or a combination thereof.

77. The composition of claim 72, wherein the dissolution agent comprises a strong base.

78. The composition of claim 77, wherein the strong base comprises lithium hydroxide, sodium hydroxide, potassium hydroxide, calcium hydroxide, strontium hydroxide, barium hydroxide, or a combination thereof.

79. The composition of claim 72, wherein the dissolution agent comprises an oxidizing agent.

80. The composition of claim 79, wherein the oxidizing agent partially or completely degrades the scaffold.

81. The composition of claim 79 or 80, wherein the oxidizing agent is sodium periodate.

82. The composition of any one of claims 58-71, wherein the scaffold comprises a biobased material and the solvent comprises an enzyme that degrades the biobased material.

83. The composition of claim 82, wherein the solvent comprises a cellulase.

84. The composition of claim 82 or 83, wherein the scaffold comprises a cellulose and the solvent comprises a cellulase.

85. The composition of claim 82, wherein the scaffold comprises an ester-containing polymer.

86. The composition of claim 82 or 85, wherein the solvent comprises an esterase.

87. The composition of any one of claims 82-86, wherein the solvent comprises a lipase.

88. The composition of claim 82, wherein the scaffold comprises a calcium alginate.

89. The composition of claim 82 or 88, wherein the solvent comprises an alginate lyase.

90. The composition of any one of claims 1 or 3-89, wherein the scaffold comprises a three-dimensional woven material.

91. The composition of claim 2 or claim 90, wherein the three-dimensional woven material comprises a spacer fabric.

92. The composition of claim 91, wherein the spacer fabric comprises a front face in contact with a filler, and a back face in contact with the filler, wherein the filler separates the front face from the back face.

93. The composition of claim 92, wherein separation of the front face from the back face by the filler creates a gap to allow nutrient entry, waste removal, cell attachment, cell growth, or any combination thereof.

94. The composition of any one of claims 2, or 90-93, wherein the three-dimensional woven material comprises a pile weave.

95. The composition of any one of claims 2, or 90-94, wherein the three-dimensional woven material comprises a terry, a frieze, a velvet, a corduroy, a velveteen, or any combination thereof.

96. The composition of any one of claims 1-95, wherein the scaffold comprises multiple layers of scaffold material.

97. The composition of claim 96, wherein the multiple layers of scaffold material comprise a combination of different form factors.

98. The composition of claim 97, wherein the different form factors comprise a non-woven material, a woven material, a needle-punched material, a three-dimensional structure, or any combination thereof.

99. The composition of any one of claims 96-98, wherein the scaffold comprises a three-layered composite material, wherein the three-layered composite material comprises two outer layers and one inner layer.

100. The composition of claim 99, wherein the two outer layers comprise a surface layer characteristic, and the inner layer comprises a bulk property.

101. The composition of any one of claims 96-100, wherein the multiple layers comprise multiple layers of a thin material.

102. The composition of any one of claims 96-101, wherein the multiple layers are fused together, held together by entanglement, laminated together, stitched together, glued together, woven together, printed together, or any combination thereof.

103. The composition of any one of claims 1-102, wherein the composition further comprises an isolated animal cell in contact with the scaffold.

104. The composition of claim 103, wherein the isolated animal cell is an isolated animal fibroblast or fibroblast-like cell.

105. The composition of any one of claims 103-104, wherein the isolated animal cell is an immortalized isolated animal cell.

106. The composition of claim 105, wherein the immortalized isolated animal cell can be grown past a Hayflick limit.

107. The composition of claim 105 or claim 106, wherein the immortalized isolated animal cell can be grown past about 40 cell divisions, about 50 cell divisions, or about 60 cell divisions.

108. The composition of any one of claims 103-107, wherein the isolated animal cell is a bovine or porcine cell.

109. The composition of any one of claims 103-107, wherein the isolated animal cell is a human cell.

110. The composition of any one of claims 1-109 wherein the extracellular matrix was produced by an isolated animal fibroblast or fibroblast-like cell.

111. The composition of any one of claims 1-110, wherein the composition is at least partially decellularized.

112. The composition of claim 111, wherein at least partially decellularized comprises substantially no intact cells being present in the composition.

113. The composition of any one of claims 1-112, wherein the scaffold further comprises a polyglycolic acid (PGA), a polybutylene succinate (PBS), a bioabsorbable synthetic polymer, a cellulose, an acetate, an acrylic, a fiber, a linen, a rayon, a velvet, a modacrylic, an olefin polyester, a saran, a vinyon, a wool, a jute, a hemp, a bamboo, a flax, or any combination thereof.

114. The composition of any one of claims 1-113, wherein the scaffold further comprises a bio-based nylon, a bio-based PET, a bio-based PEF, a bio-based polylactic acid (PLA) or any combination thereof.

115. The composition of any one of claims 1-114, wherein the scaffold comprises a nylon 1,6, a nylon 4,6, a nylon 510, a nylon 5,6, a nylon 5,12, a nylon 6, a nylon 6,6, a nylon 11, a nylon 10.10, a nylon 12, or any combination thereof.

116. The composition of any one of claims 1-115, wherein the scaffold comprises a bast fiber comprising a flax, a hemp, a linen, a jute, a ramie, a kenaf, a sisal, or any combination thereof.

117. The composition of any one of claims 1-116, wherein the scaffold has a thickness of from about 0.1 mm to about 4 mm.

118. The composition of any one of claims 1-116, wherein the scaffold has a thickness of from about 1 mm to about 3 mm.

119. The composition of any one of claims 1-116, wherein the scaffold has a thickness of about 1 mm.

120. The composition of any one of claims 1-116, wherein the scaffold has a thickness of about 2 mm.

121. The composition of any one of claims 1-116, wherein the scaffold has a thickness of about 3 mm.

122. The composition of any one of claims 1-121, wherein the scaffold comprises fibers comprising a dtex of about 6.7 dtex.

123. The composition of any one of claims 1-122, wherein the scaffold comprises fibers comprising a diameter of from about 1 μm to about 100 μm.

124. A method of making the composition of any one of claims 1-123, comprising seeding an isolated animal fibroblast or fibroblast-like cell onto the scaffold to form the composition.

125. A method of making the composition of any one of claims 1-123, wherein the scaffold comprises a thermoplastic polymer which is subsequently substantially removed from the extracellular matrix prior to a tanning.

126. The method of claim 125, wherein the thermoplastic polymer comprises a polyvinyl alcohol (PVA), and wherein the method comprises substantially removing the PVA prior to a tanning by contacting the PVA with water having a temperature of about 18° C. to about 90° C.

127. The method of claim 125, wherein the thermoplastic polymer comprises a Polylactic acid (PLA) and wherein the method comprises substantially removing the PLA prior to a tanning by contacting the PLA with a solvent to remove the PLA.

128. A method of making the composition of any one of claims 1-123, wherein the scaffold comprises a dissolvable scaffold which is substantially removed by contacting the scaffold with a solvent.

129. A method of making the composition of any one of claims 1-123, comprising needle punching the scaffold using a needle loom to entangle fibers in the non-woven scaffold material.

130. The method of claim 129, wherein the needle loom comprises a barbed needle.

131. The method of claim 129, wherein the needle punching creates pores in the scaffold material.

132. A method of making the composition of any one of claims 1-123, comprising at least partially coating the scaffold with a coating comprising a Matrigel, a vitronectin, a fibronectin, a protein extract from a soy, a protein extract from a pea, a protein extract from a corn, a peptide generated from synthesis, an RNA-binding glycine-rich (RBG) protein, a synthetic protein, an RGD peptide, a polylysine, a polyarginine, a polyornithine, a recombinant protein, an oligomer, a polymer, GTAMC, carbohydrate-binding module, a cellulose binding domain, a starch binding domain, or a combination thereof.

133. The method of claim 132, further comprising seeding an isolated animal fibroblast or fibroblast-like cell onto the scaffold to form the composition.

134. A method comprising transplanting the composition of any one of claim 1-123 onto a patient in need of a skin graft for the treatment of lost or damaged skin.

135. The method of claim 134, wherein the patient has lacerations, contusions, lesions, sores, burns, wounds, surgical wounds, surgically removed skin, necrotic skin, or any combination thereof.

136. A method comprising tanning the composition of any one of claims 1-123 to produce a cruelty free leather.

137. A cruelty-free leather produced by tanning the composition of any one of claims 1-123.

138. A method comprising:
a) seeding isolated animal fibroblasts or fibroblast-like cells onto a scaffold to form a cell layer on a scaffold, wherein the scaffold comprises a polycaprolactone (PCL), a polylactic acid (PLA), a polylactic and polyglycolic acid (PLGA), a polyethylene terephthalate (PET), a Nylon, a polyethylene (PE), a polyethylene furanoate (PEF), a polypropylene (PP), a polyvinyl alcohol (PVA), a cotton, a bast fiber, a viscose, a modal, a lyocell, a plant based protein fiber, an alginate fiber, a thermoplastic starch, or any combination thereof,
b) growing the cells to produce a composition comprising an extracellular matrix; and
c) tanning the composition comprising an extracellular matrix to form a cruelty-free leather.

139. A method of using the cruelty-free leather of any one of claims 136-137 as a substitute for a traditional leather in a leather article.

140. The method of claim 139, wherein the leather article comprises a watch strap, a belt, a suspender, a packaging, a shoe, a boot, a footwear, a glove, a clothing, a bag, a clutch, a purse, a coin purse, a billfold, a key pouch, a credit card case, a pen case, a backpack, a case, a wallet, a saddle, a harness, a whip, a luggage, a travel good, a rucksack, a portfolio, a document bag, a briefcase, an attache case, a pet article, a leash, a collar, a hunting and fishing article, a gun case, a cutlery case, a holster for a fire arm, a stationary article, a writing pad, a book cover, a camera case, a spectacle case, a cigarette case, a cigar case, a jewel case, a mobile phone holster, a sport article, a ball, a basketball, a soccer ball, a football, or any combination thereof.

141. A leather article comprising the cruelty-free leather of any one of claims 136-137.

142. The leather article of claim 141, comprised in a watch strap, a belt, a suspender, a packaging, a shoe, a boot, a footwear, a glove, a clothing, a bag, a clutch, a purse, a coin purse, a billfold, a key pouch, a credit card case, a pen case, a backpack, a case, a wallet, a saddle, a harness, a whip, a luggage, a travel good, a rucksack, a portfolio, a document bag, a briefcase, an attache case, a pet article, a leash, a collar, a hunting and fishing article, a gun case, a cutlery case, a holster for a fire arm, a stationary article, a writing pad, a book cover, a camera case, a spectacle case, a cigarette case, a cigar case, a jewel case, a mobile phone holster, a sport article, a ball, a basketball, a soccer ball, a football, or any combination thereof.

143. The method of claim 140 or the leather article of claim 142, wherein the clothing comprises a top, a bottom, an outerwear, or any combination thereof.

144. The method of claim 140 or the leather article of claim 142, wherein the bag comprises a handbag with or without shoulder strap.

145. The method of claim 140 or the leather article of claim 142, wherein the luggage comprises a trunk, a suitcase, a travel bag, a beauty case, a toilet kit, or any combination thereof.

146. A method of using the composition of any one of claims 1-123 for the treatment of a disease or ailment.

147. A kit comprising the composition of any one of claims 1-123 or the leather article of any one of claims 139-145.
